(19) United States
(12) Patent Application Publication
(10) Pub. No.: US 2008/0214666 A1
(43) Pub. Date: Sep. 4, 2008

(54) Products and Methods Related to Mono-Methyl Branched-Chain Fatty Acids

(75) Inventors: Marina Kniazeva, Boulder, CO (US); Min Han, Boulder, CO (US)

Correspondence Address: SHERIDAN ROSS PC, 1560 BROADWAY, SUITE 1200, DENVER, CO, 80202, US

(73) Assignee: The Regents of the University of Colorado, Boulder, CO (US)

(21) Appl. No.: 11/570,869

(22) PCT Filed: Jun. 17, 2005
(86) PCT No.: PCT/US05/21401

§ 371 Date: Sep. 6, 2007

Related U.S. Application Data
(60) Provisional application No. 60/580,823, filed on Jun. 18, 2004.

(51) Int. Cl. A61K 31/20; A01K 67/027; C12Q 1/02; A61P 43/00; C07C 59/00

(52) U.S. Cl. 514/558; 800/9; 435/29; 800/3; 554/1

(57) ABSTRACT

Disclosed are genes and proteins related to the biosynthesis and function of mono-methyl branched-chain fatty acids (mmBCFA) in eukaryotes, as well as the functions of mmBCFA in eukaryotic organisms. Also disclosed are methods to regulate the biosynthesis and function of mmBCFA in an organism, methods to use the valuable targets associated with mmBCFA biosynthesis and function as therapeutic agents and to screen for pharmaceuticals and nutraceuticals, or to investigate or screen for regulators of metabolism, growth, development, and reproduction in eukaryotes. The present invention also includes highly specific and useful animal models for mmBCFA biosynthesis and function that can be used to explore pharmaceutical applications of the mmBCFA-involved biological processes.

### FIELD OF THE INVENTION

The present invention generally relates to the discovery that mono-methyl branched-chain fatty acids (mmBCFA) are important for metabolism, fatty acid synthesis and homeostasis, growth, development and reproduction in eukaryotes, as well as the discovery of several genes and proteins that are involved in the biosynthesis and regulation of mmBCFA and these processes. The present invention relates to the use of these discoveries in in vitro and in vivo methods related to the regulation of these processes in eukaryotes.

### BACKGROUND OF THE INVENTION

Fatty acids (FA) belong to a physiologically important class of molecules involved in energy storage, membrane structure, and various signaling pathways. Different FA have different physical properties that determine their unique functions. Among the most abundant in animal cells as well as the most studied are those of straight long chain even numbered saturated and unsaturated fatty acids.

Mono-methyl branched chain fatty acids (mmBCFA) are commonly found in many organisms from bacteria to mammals. In humans, they have been detected in skin, brain, blood, and cancer cells. Despite a broad distribution, mmBCFA remain exotic in eukaryotes, where their origin and physiological roles are not understood.

C15ISO and C17ISO are saturated tetradecanoic and hexadecanoic FAs with a single methyl group appended on the carbon next to the terminal carbon (FIG. 1). Both ISO and anteISO mono-methyl branched-chain fatty acids (mmBCFA) also seem to be ubiquitous in nature. They are present in particularly large quantities in various bacterial genera, including cold-tolerating and thermophilic species. There, mmBCFA contribute to the membrane function regulating fluidity (Rilfors, Wieslander et al. 1978) and proton permeability (van de Vossenberg, Driessen et al. 1999).

Although comprehensive reports on mmBCFA in eukaryotes are lacking, sporadic data indicate that they are present in fungi, plant, and animal kingdoms. In mammals, mmBCFA have been detected in several tissues including skin, Verix caseosa, harderian and sebaceous glands, hair, brain, blood, and cancer cells. The fact that mmBCFA are present in a wide variety of organisms implies a conservation of the related metabolic enzymes and consequently important and perhaps unique functions for these molecules (Jones and Rivett 1997). Nevertheless, their physiological roles and metabolic regulations have not been systematically studied and thus remain fragmentary.

It has been found that C21anteISO is the major covalently bound FA in mammalian hair fibers. A removal of this FA from its protein counterparts results in a loss of hydrophobicity (Jones and Rivett 1997). Other studies indicated that C17anteISO esterified to cholesterol binds to and activates enzymes of protein biosynthesis (Tuhackova and Hradec 1985; Hradec and Dufek 1994). A potential significance of mmBCFA for human health is associated with a long observed correlation between amounts of these FAs and disease conditions such as brain deficiency (Ramsey, Scott et al. 1977) and cancer (Hradec and Dufek 1994). More recent studies have revealed a role of another mmBCFA, C15ISO, as a growth inhibitor of human cancer where it selectively induces apoptosis (Yang, Liu et al. 2000). Given how important these FA molecules may be and how little is known about their biosynthesis and functions in eukaryotes, it is an opportune problem to study.

De novo synthesis of long-chain mmBCFA described for bacteria is principally different from the biosynthesis of straight-chain FA (Oka and Kaneda 1988). While the latter uses acetyl-CoA as a primer condensing with a malonyl-CoA extender, BCFA synthesis starts with the branched-chain CoA primers derived from branched-chain amino acids; leucine, isoleucine, and valine. To synthesize BCFA, organisms must have a system of supplying branched-chain primers along with the enzymes utilizing them (Smith and Kaneda 1980). However, no such enzymes have been previously characterized in vivo in any eukaryotic organisms.

### SUMMARY OF THE INVENTION

One embodiment of the present invention relates to a non-human animal model for studying metabolism or homeostasis of mono-methyl branched-chain fatty acids (mmBCFA), the regulation of growth, the regulation of development or the regulation of reproduction in eukaryotic organisms. The non-human animal model has been modified to delete or inactivate a protein or functional homologue thereof, the protein selected from: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (RuvB-like) (SEQ ID NO:20), pantothenate kinase (PNK-1) (SEQ ID NO:22), branched-chain α-keto-acid dehydrogenase (BCKAD) α subunit (SEQ ID NO:24), BCKAD pyruvate dehydrogenase subunit (SEQ ID NO:38), and oligopeptide transporter PEP-2 (PEP-2) (SEQ ID NO:26). In one embodiment, the non-human animal is produced using RNAi targeted to RNA encoding the protein or homologue thereof. In a preferred embodiment, animal is C. elegans.

Another embodiment of the present invention relates to an isolated cell for evaluating the biosynthesis and function of mono-methyl branched-chain fatty acid (mmBCFA) in vitro, comprising a eukaryotic cell that produces mmBCFA. The cell has a modification resulting in the deletion or inactivation of at least one protein or functional homologue thereof, the protein selected from: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (RuvB-like) (SEQ ID NO:20), pantothenate kinase (PNK-1) (SEQ ID NO:22), branched-chain α-keto-acid dehydrogenase (BCKAD) α subunit (SEQ ID NO:24), BCKAD pyruvate dehydrogenase subunit (SEQ ID NO:38), and oligopeptide transporter PEP-2 (PEP-2) (SEQ ID NO:26).

Yet another embodiment of the invention relates to a method to identify compounds that regulate the biosynthesis or function of mono-methyl branched-chain fatty acids (mmBCFA) in a eukaryotic organism. The method includes identifying a compound that regulates the expression or biological activity of a C. elegans protein or a eukaryotic homologue thereof. The protein is selected from: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (SEQ ID NO:20), pantothenate kinase (PNK-1) (SEQ ID NO:22), branched-chain α-keto-acid dehydrogenase (BCKAD) α subunit (SEQ ID NO:24), BCKAD pyruvate dehydrogenase subunit (SEQ ID NO:38), and oligopeptide transporter PEP-2 (PEP-2) (SEQ ID NO:26). In one aspect, the identification of compounds that increase the expression or biological activity of the protein or homologue thereof are selected as compounds that increase the biosynthesis or function of mmBCFA. In another aspect, the identification of compounds that decrease the expression or biological activity of the protein or homologue thereof are selected as compounds that decrease the biosynthesis or function of mmBCFA.

In one aspect, the method further includes a step of assessing the ability of an identified compound to regulate the metabolism or homeostasis of mmBCFA in a non-human organism, or to regulate growth, development or reproduction in a non-human organism. In this aspect, the non-human organism can include, but is not limited to, C. elegans, a fungus, an alga, and a non-human mammal.

In another aspect, the method comprises identifying compounds that regulate mmBCFA biosynthesis or function in a eukaryotic cell that naturally synthesizes mmBCFA. The eukaryotic cell can include, but is not limited to, a nematode cell, a fungal cell, an algal cell, and a mammalian cell, and in one aspect, is a human cell. In one aspect, the method includes detecting the ability of a compound to regulate the production of an mmBCFA selected from the group consisting of C15ISO and C17 ISO.

In another embodiment of the invention, the method includes the steps of: (a) contacting a host cell with a putative regulatory compound, wherein the host cell expresses the protein or homologue thereof or a biologically active fragment thereof; and (b) detecting whether the putative regulatory compound inhibits or increases the expression or biological activity of the protein or homologue thereof or biologically active fragment thereof. A putative regulatory compound that inhibits the expression or biological activity of the protein as compared to in the absence of the compound is selected as a compound for inhibiting mmBCFA biosynthesis or function in a eukaryotic organism. In addition, a putative regulatory compound that increases the biological activity of the protein as compared to in the absence of the compound is selected as a compound for increasing mmBCFA biosynthesis or function in a eukaryotic organism. In one aspect, the expression of the protein or homologue or fragment thereof is detected by detecting the transcription of a gene encoding the protein. In another aspect, the expression of the protein or homologue or fragment thereof is detected by detecting the translation of the protein. In yet another aspect, the biological activity of the protein or homologue or fragment thereof is detected by detecting a product generated in a biochemical reaction mediated by the protein. In another aspect, the biological activity of the protein or homologue or fragment thereof is detected by detecting a substrate consumed in a biochemical reaction mediated by the target.

In another aspect of this embodiment, the method includes the steps of: (a) administering a putative regulatory compound to a non-human animal that has been modified to delete or inactivate a protein or functional homologue thereof, the protein selected from: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (RuvB-like) (SEQ ID NO:20), pantothenate kinase (PNK-1) (SEQ ID NO:22), branched-chain α-keto-acid dehydrogenase (BCKAD) α subunit (SEQ ID NO:24), BCKAD pyruvate dehydrogenase subunit (SEQ ID NO:38), oligopeptide transporter PEP-2 (PEP-2) (SEQ ID NO:26); phosphoinositide-dependent protein kinase 1 (PDK-1) (SEQ ID NO:28); and insulin receptor DAF-2 (DAF-2) (SEQ ID NO:30); (b) detecting a change in the non-human animal in the presence of the compound as compared to in the absence of the compound, the change being selected from: (i) an increase or decrease in the expression or biological activity of the protein or homologue thereof; (ii) an increase or decrease in the amount or type of mmBCFA synthesized by the non-human animal; (iii) a change in the total fatty acid profile of the non-human animal; (iv) an increase or decrease in insulin-signaling in the non-human animal; (v) a change in embryogenesis in the non-human animal or progeny thereof; (vi) a change in the fertility of the non-human animal or progeny thereof; (vii) a change in the viability of progeny of the non-human animal; (viii) an increase or decrease in the growth or development of the non-human animal or progeny thereof; and/or (ix) a change in a metabolic response to food sensation in the non-human animal; and (c) selecting a compound as a compound that regulates mmBCFA biosynthesis or function if a change is detected in (b) in the presence of the compound as compared to the absence of the compound. In a preferred embodiment, the non-human animal is a C. elegans.

In one aspect of this embodiment, the non-human animal has a modification that results in the deletion or inactivation of ELO-5, and detection of a compound that increases the biosynthesis of mmBCFA selected from the group consisting of C15ISO and C17ISO is selected in step (c). Alternatively, detection of a compound that increases the growth and development of the non-human animal and progeny thereof is selected in step (c). Alternatively, detection of a compound that regulates insulin-signaling is selected in step (c).

In another aspect, of this embodiment, the non-human animal has a modification that results in the deletion or inactivation of ACS-1, and detection of a compound that restores functional embryogenesis to the non-human animal is selected in step (c). Alternatively, detection of a compound that increases the biosynthesis of mmBCFA selected from the group consisting of C15ISO and C17ISO is selected in step (c).

In another aspect, the non-human animal has a modification that results in the deletion or inactivation of LPD-1, and detection of a compound that increases the biosynthesis of mmBCFA selected from the group consisting of C15ISO and C17ISO is selected in step (c).

In another aspect, the non-human animal has a modification that results in the deletion or inactivation of ACS-1 and ELO-5, and detection of a compound that increases growth and development of the progeny of the non-human animal is selected in step (c).

In another aspect, the non-human animal has a modification that results in the deletion or inactivation of RuvB-like protein or a homologue thereof, and detection of a compound that decreases monounsaturated fatty acid levels in the non-human animal is selected in step (c).

In yet another aspect, the non-human animal has a modification that results in the deletion or inactivation of PNK-1, and detection of a compound that increases the biosynthesis of mmBCFA selected from the group consisting of C15ISO and C17ISO is selected in step (c).

In another aspect, the non-human animal has a modification that results in the deletion or inactivation of NHR-49, and detection of a compound that decreases saturated fatty acid levels in the non-human animal is selected in step (c).

In another aspect, the non-human animal has a modification that results in the deletion or inactivation of BCKAD, and detection of a compound that increases the biosynthesis of mmBCFA selected from the group consisting of C15ISO and C7ISO is selected in step (c).

In yet another aspect, the non-human animal has a modification that results in the deletion or inactivation of ELO-6, and detection of a compound that increases the biosynthesis of C17ISO is selected in step (c).

In any of the above aspects, the method can further comprise, either before, during or after step (a) or (b), a step of providing an exogenous mmBCFA selected from: C13ISO, C15ISO, C17ISO, C15ante-ISO, C17-anteISO, and a methyl ester thereof, wherein the method further comprises a step of detecting a change in the non-human animal in the presence and absence of the exogenous mmBCFA.

Another embodiment of the present invention relates to a formulation comprising at least one mono-methyl branched-chain fatty acid (mmBCFA) selected from: C13ISO, C15ISO, C17ISO, C15ante-ISO, and C17-anteISO, or a functional derivative of any of the mmBCFA or a methyl ester of any of the mmBCFA, or combinations thereof. In one aspect, the mmBCFA is selected from C15ISO, C17ISO, C15ante-ISO, and C17-anteISO, or a methyl ester thereof. In another aspect, the mmBCFA is selected from C17ISO and C17-anteISO, or a methyl ester thereof. In another aspect, the formulation is a dietary supplement, which can further include at least one additional dietary agent selected from the group consisting of a vitamin, a mineral, a protein, a carbohydrate, and a lipid. In one aspect, the formulation is a nutraceutical formulation. In another aspect, the formulation is a pharmaceutical formulation, which can further include at least one agent for the treatment of a disease or condition, or a symptom thereof, wherein the disease or condition is associated with metabolism, growth, development or reproduction of a eukaryotic organism. In any of these aspects, the formulation can include a pharmaceutically acceptable excipient and/or can be provided in a form suitable for oral administration.

Another embodiment of the present invention relates to a method to increase mono-methyl branched-chain fatty acid (mmBCFA) in a eukaryotic organism. The method includes the step of administering to the organism at least one mono-methyl branched-chain fatty acid (mmBCFA) selected from: C13ISO, C15ISO, C17ISO, C15ante-ISO, and C17-anteISO, or a functional derivative of any of the mmBCFA or a methyl ester of any of the mmBCFA, or combinations thereof. In one aspect, the organism has a disease or condition associated with a deficiency of mmBCFA. In another aspect, the mmBCFA is administered in a dietary supplement formulation.

Yet another embodiment of the present invention relates to a method to regulate the biosynthesis of mono-methyl branched-chain fatty acids (mmBCFA) in a eukaryotic organism. The method includes regulating the expression or biological activity of a protein or functional homologue thereof, wherein the protein is selected from: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), pantothenate kinase (PNK-1) (SEQ ID NO:22), branched-chain α-keto-acid dehydrogenase (BCKAD) α subunit (SEQ ID NO:24), BCKAD pyruvate dehydrogenase subunit (SEQ ID NO:38), and oligopeptide transporter PEP-2 (PEP-2) (SEQ ID NO:26). In one aspect, the method includes increasing the expression or biological activity of the protein by overexpressing a gene encoding the protein in the cells of the organism. In another aspect, the method includes inhibiting the expression or biological activity of the protein or homologue thereof by inhibiting the transcription of RNA encoding the protein.

Another embodiment of the invention relates to a method to treat a patient with Maple Syrup Urine Disease, comprising administering to a patient with Maple Syrup Urine Disease at least one mono-methyl branched-chain fatty acid (mmBCFA) selected from: C13ISO, C15ISO, C17ISO, C15ante-ISO, and C17-anteISO, or a functional derivative of any of the mmBCFA or a methyl ester of any of the mmBCFA, or combinations thereof.

Yet another embodiment of the invention relates to a method to regulate or evaluate insulin-signaling in a eukaryotic organism, comprising regulating in the organism the level of at least one mono-methyl branched-chain fatty acid (mmBCFA) selected from the group consisting of: C13ISO, C15ISO, and C17ISO, wherein the step of regulating mmBCFA regulates insulin-signaling, fat storage, or growth and development of the organism. In one aspect, the step of regulating comprises administering to the organism at least one mmBCFA selected from the group consisting of, C13ISO, C15ISO, and C17ISO, C15ante-ISO, and C17-anteISO, or a functional derivative of any of the mmBCFA or a methyl ester of any of the mmBCFA, or combinations thereof. In another aspect, the step of regulating comprises depleting at least one mmBCFA in the animal. In this aspect, the step of regulating can include inhibiting the expression or activity of at least one protein associated with the biosynthesis of mmBCFA in the organism.

### BRIEF DESCRIPTION OF THE DRAWINGS OF THE INVENTION

FIG. 1 is a diagram showing the structure of mmBCFA of 15 and 17 carbons (C15ISO, 13-methyl myristic acid; C17ISO, 15-methyl hexadecanoic acid; C17anteISO, 14-methyl hexadecanoic acid).

FIGS. 2A-2D show that RNAi treatment of elo-5 and elo-6 significantly alters the fatty acid (FA) composition in Caenorhabditis elegans strains. FIGS. 2A and 2B show gas chromatography (GC) profiles showing the FA composition in wild type strain (Bristol N2) containing the RNAi feeding control vector and in the elo-5(RNAi) feeding strain. FIG. 2C shows a comparison of FA composition in three strains; wild type, elo-5(RNAi) and elo-6(RNAi). FIG. 2D shows the elongation reactions catalyzed by ELO-5 and ELO-6 in the C15ISO and C17ISO biosynthesis.

FIGS. 3A-3D show that the C. elegans BCKAD homolog is involved in mmBCFA biosynthesis. FIG. 3A shows the early steps of the mmBCFA biosynthesis in bacteria, based on (Oku and Kaneda 1988); FIG. 3B shows GC profiles that reveal differences in the FA composition in the wild type and animals treated with RNAi of E1 alpha subunit of BCKAD encoded by Y39E4A.3; FIG. 3C shows a summary of several independent preparations indicating a significant decrease in both mmBCFA in the Y39E4A.3 dsRNA-treated animals.

FIGS. 4A-4E show the fatty acid (FA) composition in worms maintained on the elo-5 RNAi plates supplemented with mmBCFA or with S. maltophilia enriched with C15ISO and C15anteISO mmBCFA (black arrowheads indicate positions of mmBCFA).

FIGS. 5A-5B shows the effects of a fluctuation of the C17ISO amounts in development.

FIG. 6 shows the correlation between the level of C17ISO and the levels of linoleic and vaccenic acids during development.

FIGS. 7A-7C show that RNAi of the C. elegans SREBP homolog alters the FA composition.

FIGS. 8A-8E show that RNAi of four candidate genes with altered expression in elo-5(RNAi) worms affects the FA composition.

FIGS. 9A and 9B are microphotographs showing an abnormal acs-1(RNAi)+C13ISO cuticle.

FIGS. 10A and 10 B show that mmBCFA biosynthesis is tightly liked to dietary protein up-take.

### DETAILED DESCRIPTION OF THE INVENTION

The present invention generally relates to the present inventors' discoveries that have revealed significant information regarding the function and regulation of mmBCFA in the eukaryotes, and particularly, in C. elegans. Specifically, the inventors show herein that C. elegans synthesizes mmBCFA de novo using metabolites of leucine degradation as precursors and utilizes the long chain fatty acid elongation enzymes, ELO-5 and ELO-6, to produce long chain mmBCFA. The biosynthesis of long chain mmBCFA depends on activation of their precursor by mmBCFA-specific acetyl-CoA synthetase (ACS-1).

The inventors have further discovered that mmBCFA are essential for C. elegans growth and development, and both ELO-5 and ACS-1 are essential for their production. Animals fully depleted of mmBCFA at the time of hatching develop severe morphological defects observed in all organs and prematurely die as small sick larvae or adults. This phenotype is reversible provided that mmBCFAs are available in a food.

The inventors have also discovered that mmBCFA with different length carbon backbones have different rescue abilities. The shorter C13 and C15 fatty acids (FA) rescue depleted larvae to wild type adults, whose progeny stops development at the first larval stage, L1. The arrest is reversible and can be overcome by feeding the arrested animals with C17 mmBCFA. The ability of C13, C15, and C17 to rescue the mmBCFA-depleted phenotype requires the activity of ACS-1. In the absence of the ACS-1 function, all three FA species rescue mmBCFA-depleted animals to wild type fertile adult, but their progeny die as early embryos.

The inventors have also shown that the correlation between amounts of the supplement and rescue ability is nonlinear in the C13ISO experiments. Specifically, concentrations of C13ISO in a range 0-0.5 mM does not rescue elo-5-depleted animals, at concentrations above 0.75 mM, 100% of animals reach adulthood and have progeny. The growth and maturation rate in these cases are equal to the growth and maturation of the wild-type animals on the similarly supplemented plates. A further increase in concentration of the supplement (to 2.5-10 mM) results in slowing at the L4 stage accompanied by a delay the adult maturation. However, the developmental delay does not seem to be harmful. To the contrary, the animals are healthy, continue to actively lay eggs in contrast to controls, and maintain a wild type brood size. This ability of C13ISO to modulate growth rate between L4 and adult stage is shared by C15ISO and C17ISO, but not by saturated and monounsaturated FA of the 16-18 carbon backbone, and it is not related to gross changes in FA composition of total lipids.

The inventors have also discovered that ACS-1 is required for embryogenesis. Animals depleted of functional ACS-1 experience a failure in cellularization resulting in multinucleated blastomeres (polyploidy) and eventually in embryonic lethality. This embryonic lethality can be partially rescued by the mutation in zen-4, encoding a homologue of mammalian kinesin-like protein-1, suggesting a genetic interaction between zen-4 and acs-1. Furthermore, the data provided herein show that a suppression of acs-1 affects formation of eggshell and adult cuticle in C. elegans. The inventors show that ACS-1 determines the architecture of cuticle and its physical properties (osmotic resistance and withstanding a mechanical pressure).

The inventors have also discovered a relationship between mmBCFA, the DAF pathway (insulin signaling pathway), and food signaling in eukaryotic cells. Specifically, the inventors show that there is a genetic interaction between acs-1, essential for mmBCFA synthesis, and DAF insulin/TGF beta pathway possibly up-stream of DAF-9 (cytochrome P450) in regulation of molting. A deficiency of mmBCFA activates the expression of targets of the transcriptional regulator DAF-16 (pnk-1 and sod-3), and stimulates the nuclear translocation of DAF-16. Therefore, mmBCFA deficiency appears to upregulate the DAF-2/DAF-16 pathway. Furthermore, elo-5 is down-regulated in animals that are depleted of functional K01G5.1, which encodes a predicted transcription factor that may bind to DAF-16. In addition, L1 arrest caused by mmBCFA deficiency precedes (in developmental scale) the L1 arrest in mid-L1 stage caused by starvation, but C17ISO, in conjunction with a bacterial feeding, rescues the animals to normal growth and development. In the absence of the mmBCFA, the animals can feed on the bacteria, but are unable to process the “food signal” that initiates growth and development in wild type larvae. Since L1 animals are not competent for dauer formation, these data indicate that mmBCFA interferes with the insulin/DAF signaling that is not related to dauer formation. Moreover, a deficiency of mmBCFA stimulates nuclear translocation of DAF-16. Therefore, mmBCFA are believed to regulate the food sensation and food processing system.

The connection of mmBCFA to the insulin-signaling pathway is further confirmed by studies showing that a deficiency of mmBCFA prevents a temperature-sensitive mutant that normally forms dauers at 20° C. and 25° C., from a proper transition into the dauer. Dauer formation in C. elegans is an adaptive response to unfavorable conditions such as overcrowding or starvation. In this state, metabolism is dramatically shifted toward energy storage as opposed to growth and development. daf-2 mutants tend to form dauers at restrictive temperatures, and the data described above now show that a deficiency of mmBCFA can interfere with the dauer formation.

The inventors have also shown that biosynthesis of mmBCFA is tightly linked to protein uptake. Specifically, functional inhibition of an oligopeptide transporter, which results in a reduction in protein uptake by the animals, resulted in a dramatic change in the FA composition of total lipids obtained from the animals. Particularly affected were the C15ISO and C17ISO fractions of the mmBCFA, which were significantly decreased in the mutant animals. The inventors have shown that this decrease is due not only to lack of substrate availability, but also to a transcriptional suppression of the mmBCFA elongation gene, elo-5. The transcriptional control over elo-5 was further shown to be mediated by the TOR pathway, which regulates gene expression by nutrient sensing. Therefore, biosynthesis of mmBCFA is sensitive to dietary protein uptake, further confirming the association of mmBCFA with signaling related to food intake and processing.

The inventors have further shown that exogenous mmBCFA affects the expression of the pantothenate kinase (PNK-1) gene that is essential for CoA biosynthesis. Specifically, while a deficiency of mmBCFA causes upregulation of pnk-1, and downregulation of pnk-1 results in decreased mmBCFA biosynthesis, high levels of mmBCFA act as a negative feedback control and downregulate the expression of pnk-1, establishing a link between mmBCFA and CoA metabolism, which is essential for energy production. The combined discoveries that mmBCFA metabolism is responsive to exogenous protein up-take and the ability of mmBCFA to influence CoA biosynthesis further indicate a role for mmBCFA in coordination of the food signaling and energy expenditure.

Finally, the inventors have shown that mammalian cells are able to elongate C13ISO into C15ISO and C17ISO in vitro. Therefore, these cell lines can be used to evaluate the physiological effect of mmBCFA, to identify mmBCFA-related enzymes and mmBCFA signaling system in mammals, and to identify regulators of these processes.

Combining genetics and biochemistry, the present inventors have identified several key enzymes and regulatory proteins that are involved in biosynthesis and homeostasis of specific fatty acids that play critical roles in animal growth and development. More specifically, the present inventors have discovered that depletion of mmBCFA affects the expression of several genes, and the activities of some of these genes affect the biosynthesis of mmBCFA, suggesting a potential feedback regulation. One of the genes, lpd-1, encodes a homologue of a mammalian sterol regulatory element binding protein (SREBP 1c). The inventors have also obtained results that indicate that elo-5 and elo-6 may be transcriptional targets of LPD-1. The inventors have further discovered that a key enzyme of the coenzyme A biosynthesis, pantothenate kinase (PNK-1), modulates the mmBCFA but not other FA quantity.

The mmBCFA-specific elongases and acetyl-CoA ligase as well as the revealed feedback regulation network provide valuable targets for therapeutic agents. The C. elegans systems described herein are also useful for screening for pharmaceuticals and nutraceuticals. Such systems can also be used to investigate or screen for regulators of metabolism, growth, development, and reproduction in eukaryotes. The present invention provides a foundation to build up an extraordinary experimental in vivo system that is both specific and discrete. Similar approaches (genetic manipulation and/or dietary supplementation) may be used on mammalian cell or other organism systems to explore pharmaceutical applications of the mmBCFA-involved biological processes.

Specifically, in one aspect of the invention, by applying a simple and efficient RNAi-feeding technique alone or with available knockout mutations, the inventors can shut down key enzymes of this particular type of long chain FA biosynthesis and activation. This results in depletion of internal source of these FA and ultimately in severe metabolic and morphological defects and premature death. By using a combination of the mutants, RNAi-targeted genes, and various dietary FA supplements, the inventors can rescue animals to discrete developmental stages: wild type adults, early embryos, and first larvae stage, as well as to full growth and proliferation.

A unique advantage of the present invention is in the versatile nature of the system of the present invention (described in detail below). It can be used for various tasks (e.g., targeting lipid metabolism, cell division (embryonic), growth and differentiation (postembryonic) as well as the food response, and more).

In addition, because this particular type of FA (mmBCFA) is found in humans, the system is also relevant to human health. Therefore, a variety of formulations, for use as dietary supplements, nutraceuticals or pharmaceutical formulations, are encompassed by the invention, as well as various strategies for impacting food sensing and insulin regulation, growth and development, reproduction, metabolism and homeostasis, and/or diseases associated with mmBCFA-deficiency or overproduction.

Prior to the present invention, intensive studies on mmBCFA have been only done in bacteria where mmBCFA biosynthesis and structural roles in membrane fluidity were shown. Indeed, although mmBCFA were first mentioned in 1823 (M.-E. Chevreul “The constitution of FAT”), and many lipid biologists have been working on the FA since then, no one has shown the dramatic roles of C13, C15, and C17ISO as described herein. The present inventors are believed to be the first to describe any non-structural function of mmBCFA. In addition, the biosynthetic system for mmBCFA has never been described for eukaryotic cells prior to the present invention, and enzymes participating in this process, including mmBCFA-specific elongases and CoA ligases were not previously recognized and characterized with regard to the mmBCFA biosynthesis. Furthermore, regulatory elements of mmBCFA biosynthesis were not previously known. The involvement of SREBP and pantothenate kinase in regulating mmBCFA biosynthesis were not known, nor the existence of elo-5 and elo-6 as the targets of SREBP. In addition, to the best of the present inventors' knowledge, prior to the present invention, the function of mmBCFA in regulating animal growth, development, and reproduction, or its use in controlling these functions by dietary or genetic manipulation have not been described. Furthermore, the role of mmBCFA in food sensing and insulin signaling regulation have not previously been described. The physiological roles of C13ISO, C15ISO and C17ISO on the whole organism level have also never been mentioned in relation to eukaryotes, and the physiological differences between mmBCFA of different lengths of carbon backbone have never been reported.

In addition, the inventors have shown that not only C13ISO, C15ISO, and C17ISO, but also their methyl esters are biologically active. Added as food supplements, they rescue mmBCFA deficiency. Moreover, unlike C13ISO and C15ISO, the C13ISO-methyl ester and C15ISO-methyl ester supplements can be efficiently elongated to C17ISO. mmBCFA-methyl esters have better solubility in physiological solutions such as culture media and buffered-saline and possibly better permeability that mmBCFA that allows better absorption through the intestine. Therefore, addition of these compounds is physiologically equivalent to the C17ISO supplements.

Dietary mmBCFA are readily absorbed by animal cells. They incorporate into various lipid fractions (phospholipids and triacylglycerols) and, therefore, may be used as physiologically active supplements. Moreover, mmBCFA have no toxic effect in eukaryotic organisms such as C. elegans in concentrations up to at least 10 mM. mmBCFA are easily dissolved in 1% NP40 and DMSO. mmBCFA may be extracted from many bacterial species naturally producing ISO- and ante-ISO forms of mmBCFA, for example, or may be produced recombinantly. In addition, mmBCFA-carrying bacteria, such as Stenotrophomonas maltophilia, may be used directly as sources of mmBCFA, without the need to purify, produce, or isolate the mmBCFA from the bacteria.

Fatty acid methyl esters are used extensively as intermediates in the manufacture of detergents, emulsifiers, wetting agents, stabilizers, textile treatments, and waxes among other applications. Lesser volumes of fatty acid methyl esters are used in a variety of direct and indirect food additive applications, including the dehydration of grapes to produce raisins, synthetic flavoring agents, and in metal lubricants for metallic articles intended for food contact use. Fatty acid methyl esters are also used as intermediates in the manufacture of a variety of food ingredients. Methyl esters, including methyl myristate, methyl palmitate, methyl palmitoleate, methyl stearate, methyl oleate, methyl linoleate, methyl docosahexanoate, methyl ecosapentanoate are cleared by the FDA as a supplementary source of fat for animal feed under 21 CFR 573.640. However, a potential physiological value of mmBCFA methyl esters has never been reported. The use of methyl esters of mmBCFA as any of the above-mentioned intermediates and/or direct and indirect food additive applications or supplement uses is also encompassed by the present invention.

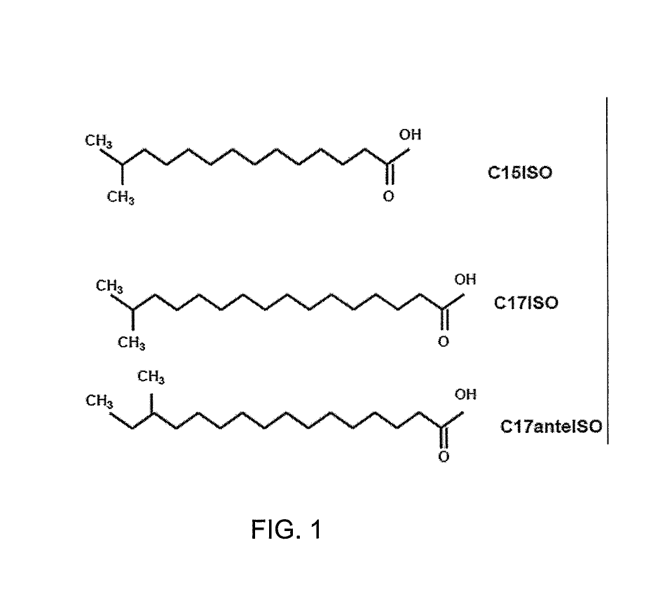

The inventors have also discovered that anteISO branched-chain FA, which cannot be synthesized by C. elegans, have a physiological potency similar to the ISO branched-chain FA. anteISO FA differ from ISO FA in a position of a single methyl attached to the third carbon from the terminal (FIG. 1). anteISO FA are abundant among bacterial FA.

One embodiment of the present invention relates to a model system for identifying, detecting, characterizing, and/or evaluating the regulation of mmBCFA (and their derivatives), for the purpose of evaluating and/or regulating processes associated with mmBCFA described herein, including, but not limited to, metabolism and/or homeostasis, organism growth, organism development, organism reproduction, food sensing and/or insulin signaling. The system includes non-human organisms or cells (from any organism, including human cells) in which the expression and/or bioactivity of at least one component (e.g., at least one gene or at least one enzyme or other protein) of an mmBCFA biosynthetic pathway has been modified so that the effects of the modification on mmBCFA (and their derivatives) and functions related thereto can be evaluated, and/or so that the effect of various regulatory agents on mmBCFA synthesis and metabolism can be evaluated. The components of an mmBCFA biosynthetic pathway can include any gene or portion thereof encoding any protein or domain or portion thereof that participates directly or indirectly in the mmBCFA biosynthetic pathway such that modification (e.g., upregulation or downregulation) of such a component has a detectable effect on mmBCFA biosynthesis. The inventors describe herein various components related to mmBCFA biosynthesis and function, using nomenclature from Caenorhabditis elegans. However, it is to be expressly understood that the discussion of genes and proteins involved in the biosynthesis and function of mmBCFA herein is not limited to C. elegans genes and proteins, but rather encompasses any functional homologue thereof from other eukaryotic organisms, and particularly from mammalian organisms, and most particularly, from humans. Therefore, the invention includes various specifically defined genes and proteins, functional homologues thereof from other eukaryotes (e.g., orthologs and other homologues that have the same specific biological activity as the reference protein), and biologically active portions (fragments) of such genes and proteins (described in more detail below). Components that have been identified by the present inventors as participating in the mmBCFA biosynthesis (as identified by the C. elegans counterparts) include, but are not limited to, the long chain fatty acid elongation enzymes ELO-5 and ELO-6, mmBCFA-specific acetyl-CoA synthetase (ACS-1), LiPid Depleted 1 (LPD-1, homologue of SREBP), nuclear hormone receptor 49 (NHR-49), RuvB-like DNA binding protein, pantothenate kinase (PNK-1), branched-chain α-keto-acid dehydrogenase (BCKAD), and any homologues or derivatives thereof, including homologous enzymes or proteins in various species having different nomenclature.

In one embodiment of the invention, a homologue is a functional homologue of the reference protein. The invention also includes orthologs of the proteins described herein. These terms are described in detail below. In one embodiment, a homologue includes a protein that is encoded by a nucleic acid molecule that hybridizes under low, moderate, high or very high stringency conditions to a nucleic acid molecule encoding a protein described herein. In another embodiment, a homologue includes a protein that is at least about 30%, 35%, 40%, 45%, 50%, 55%, 60%, 65%, 70%, 75%, 80%, 85%, 90%, or at least about 95% identical to a protein described herein, or any increment between 30% and 99%, in whole percentage increments (31%, 32%, 33%, etc.). Methods for determining hybridization conditions and percent identity are discussed in detail below.

The system can also include organisms or cells having an intact mmBCFA biosynthesis capacity, whereby the effects of putative regulators on the “wild-type” or naturally occurring system can be evaluated. Functional and non-functional mmBCFA systems can be used in combination to fully evaluate the effects of various manipulations, components and putative regulatory compounds.

The organisms to be modified include any organisms that naturally produce mmBCFA or any organisms that can be genetically modified to produce mmBCFA, including, but not limited to, bacterial cells and eukaryotic organisms, the eukaryotic organisms including, but not limited to, C. elegans, insect cells and systems, and mammals (non-human, unless the goal of the genetic modification is a gene therapy approach). Eukaryotic cells (rather than whole organisms) can also be used, including, but not limited to, any fungal cells (e.g., yeast), insect cells, algal cells, and mammalian cells (including human cells).

As used herein, a genetically modified organism can include any organism having a genome that is modified (i.e., mutated or changed) from its normal (i.e., wild-type or naturally occurring) form such that the desired result is achieved (i.e., increased, decreased or otherwise modified mmBCFA activity and/or production of a desired product using the mmBCFA system). Genetically modified organisms also include organisms having an intact or unmodified genome but have been modified by the introduction of additional genetic elements that remain extrachromosomal yet exert an effect on the organism (e.g., by the addition of RNAi that inhibits or silences the RNA encoding the protein, or by expression of an exogenous protein that exerts an effect on the mmBCFA system). Genetic modification of an organism can be accomplished using classical strain development and/or molecular biological techniques. Such techniques known in the art and are generally disclosed in the art. A genetically modified organism can include an organism in which nucleic acid molecules have been inserted, deleted or modified (i.e., mutated; e.g., by insertion, deletion, substitution, and/or inversion of nucleotides), in such a manner that such modifications provide the desired effect within the organism (e.g., deletion or inactivation of a gene or a protein encoded by the gene).

In one embodiment, the organism is modified to delete or inactivate a protein that is involved with or associated with the biosynthesis or function of mmBCFA as described herein. The deletion or inactivation can be achieved by any suitable method, and can be accomplished at the DNA level (deletion or inactivation of the gene encoding the protein or mutation of the gene so that an inactive protein is produced), at the RNA level (inhibition, silencing or elimination of the RNA encoding the protein or mutation of the RNA so that an inactive protein is produced) or at the protein level (by deletion or inactivation of the protein itself). Such proteins include any of the proteins described herein that are associated with mmBCFA biosynthesis or function, or that can affect or be used to evaluate mmBCFA biosynthesis or function (e.g., by the creation of organisms that are deficient in or overexpress such proteins), including, but not limited to: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (RuvB-like) (SEQ ID NO:20), pantothenate kinase (PNK-1) (SEQ ID NO:22), branched-chain α-keto-acid dehydrogenase (BCKAD) (α subunit-SEQ ID NO:24; pyruvate dehydrogenase subunit-SEQ ID NO:38), oligopeptide transporter PEP-2 (PEP-2) (SEQ ID NO:26); phosphoinositide-dependent protein kinase 1 (PDK-1) (SEQ ID NO:28), insulin receptor DAF-2 (DAF-2) (SEQ ID NO:30), ZEN-4 (SEQ ID NO:32), POD-1 (SEQ ID NO:34), and SOD-3 (SEQ ID NO:36).

The present inventors have described a particularly suitable method for the modification of organisms to selectively modify the expression or production of particular enzymes and proteins in the model C. elegans system. This method uses RNA interference and specifically, the feeding of RNAi to C. elegans to interfere with the expression of specific components of the mmBCFA biosynthetic pathway and thereby disrupt various aspects of mmBCFA biosynthesis and corresponding biological activities of mmBCFA and their derivatives including growth, development and reproduction of the organism. RNA interference (RNAi) is a process whereby double stranded RNA, and in mammalian systems, short interfering RNA (siRNA), is used to inhibit or silence expression of complementary genes. In the target cell, siRNA are unwound and associate with an RNA induced silencing complex (RISC), which is then guided to the mRNA sequences that are complementary to the siRNA, whereby the RISC cleaves the mRNA. The C. elegans system useful in the present invention is described in detail in the Examples section.

PCT Publication WO 00/76308, incorporated herein by reference in its entirety, describes the use of invertebrate systems to elucidate biochemical pathways associated with SREBP. This publication also illustrates various techniques for manipulation of in vivo systems that can be used in the present invention. The present invention allows for the use of such systems to elucidate biochemical and biological pathways associated with mmBCFA, and provides extensive detail regarding components of the system. The SREBP expression system described in PCT Publication WO 00/76308 can be incorporated into the system and methods of the present invention to develop sophisticated methods for screening for regulators of mmBCFA biosynthesis, metabolism and homeostasis and associated biological/physiological processes. Alternatively, a combination of mmBCFA-related genetic engineering and dietary supplements can be used to separate SREBP functions associated with the mmBCFA production and its other biological roles. This “divide and conquer” approach may provide a specific tool for determining how the SREBP activities in the various pathways interplay.

The present inventors have also demonstrated the use of this system to evaluate the effects of adding a compound into the system, such as by replacing mmBCFA as a dietary supplement. Therefore, another embodiment of the present invention relates to methods to evaluate potential pharmaceutical, nutraceutical or dietary compounds and formulations for effects on mmBCFA metabolism and/or homeostasis (including food sensation and insulin signaling) or for effects on physiological systems related thereto, including growth, development and/or reproduction. For example, one may use the systems of the invention to provide an organism with impaired mmBCFA biosynthesis which may or may not result in impaired or altered growth, development and/or reproduction, and then test putative regulatory compounds to determine whether such compounds compensate for, correct, enhance or otherwise modify the phenotype of the organism or any genetic, biochemical or physiological characteristic, as compared to in the absence of the compound. Similarly, one may use the system of the invention to screen for the effects of various compounds and formulations on the intact or wild-type mmBCFA system or components thereof (genes and proteins encoded thereby) in a specific manner, now that many of the components in the system are known. Importantly, the various combinations of modifications and organisms can be used to construct many elegant and highly useful and specific systems for pharmaceutical and/or nutraceutical applications related to mmBCFA-associated biological processes. Such systems will be apparent to those of skill in the art given the disclosure provided herein and are encompassed by the present invention.

Another embodiment of the present invention relates to the use of any of the components of the mmBCFA biosynthetic and metabolic system described herein as targets for the design and/or identification of pharmaceutical or nutraceutical compounds that regulate mmBCFA metabolism or homeostasis and/or biological functions associated with mmBCFA such as growth, development and/or reproduction. Such targets can also be used in methods to evaluate various biological, biochemical, and/or genetic processes related to mmBCFA functions and related physiological activities.

Specific targets of the present invention are described below in the Examples section and include, but are not limited to (referenced using C. elegans nomenclature and sequences, but intended to include other eukaryotic homologues (orthologs)), long chain fatty acid elongation enzymes ELO-5 (nucleic acid sequence represented by SEQ ID NO:9, encoding an amino acid sequence of SEQ ID NO:10) and ELO-6 (nucleic acid sequence represented by SEQ ID NO:11, encoding an amino acid sequence of SEQ ID NO:12), mmBCFA-specific acetyl-CoA synthetase (ACS-1) (nucleic acid sequence represented by SEQ ID NO:13, encoding an amino acid sequence of SEQ ID NO:14), LiPid Depleted 1 (LPD-1, the SREBP homolog) (nucleic acid sequence represented by SEQ ID NO:15, encoding an amino acid sequence of SEQ ID NO:16), nuclear hormone receptor 49 (NHR-49) (nucleic acid sequence represented by SEQ ID NO:17, encoding an amino acid sequence of SEQ ID NO:18), RuvB-like DNA binding protein (nucleic acid sequence represented by SEQ ID NO:19, encoding an amino acid sequence of SEQ ID NO:20), pantothenate kinase (PNK-1) (nucleic acid sequence represented by SEQ ID NO:21, encoding an amino acid sequence of SEQ ID NO:22), branched-chain α-keto-acid dehydrogenase (BCKAD) (nucleic acid sequence encoding the α subunit represented by SEQ ID NO:23, encoding an amino acid sequence of SEQ ID NO:24; nucleic acid sequence encoding the pyruvate dehydrogenase subunit represented by SEQ ID NO:37, encoding an amino acid sequence of SEQ ID NO:38), Zygotic epidermal Enclosure defective (ZEN-4) (nucleic acid sequence represented by SEQ ID NO:31, encoding an amino acid sequence of SEQ ID NO:32), DAF-2 (insulin receptor) (nucleic acid sequence represented by SEQ ID NO:29, encoding an amino acid sequence of SEQ ID NO:30), phosphoinositide-dependent protein kinase 1 (PDK-1) (nucleic acid sequence represented by SEQ ID NO:27, encoding an amino acid sequence of SEQ ID NO:28), PEP-2 (nucleic acid sequence represented by SEQ ID NO:25, encoding an amino acid sequence of SEQ ID NO:26), POD-1 (nucleic acid sequence represented by SEQ ID NO:33, encoding an amino acid sequence of SEQ ID NO:34), and SOD-3 (nucleic acid sequence represented by SEQ ID NO:35, encoding an amino acid sequence of SEQ ID NO:36). However, it will be clear to those of skill in the art that additional targets can be determined or used given the disclosure and discussion of the invention provided herein. The targets include the genes and products of the genes or any useful portion thereof that participate directly or indirectly in an aspect of mmBCFA biosynthesis and/or function. These targets also include any homologous proteins, and particularly, homologous proteins that have the same or essentially the same function, from other eukaryotic species (orthologs). Methods of the present invention for identifying therapeutic or nutraceutical compounds by identifying a regulator (e.g., an inhibitor, enhancer or inducer) of a target include identifying a regulator of any of the target genes described or contemplated herein, as well as target products encoded by any of the foregoing. The nucleic acid and amino acid sequences for genes and their encoded proteins described herein are known in the art and can also be readily determined and isolated by one of skill art given the disclosure provided herein.

All of the C. elegans nucleic acid sequences and proteins identified herein and represented by a sequence herein are described in full in the WormBase database (Chen et al., (2005). WormBase: a comprehensive data resource for Caenorhabditis biology and genomics Nucleic Acids Research 33:D383-D389; Harris et al., (2004). WormBase: a multi-species resource for nematode biology and genomics Nucleic Acids Research 32:D411-D417; Harris et al., (2003). WormBase: a cross-species database for comparative genomics. Nucleic Acids Research 31:133-137; and Stein et al., (2001). WormBase: network access to the genome and biology of Caenorhabditis elegans. Nucleic Acids Research 29:82-86). In the WormBase database, these genes and proteins are identified by nucleotide and protein sequence, name, function, gene models, Pfam domains, gene ontology, and alleles, and homologous sequences and orthologs are identified. The information provided by the WormBase accession numbers (sequence name) provided herein is incorporated by reference in its entirety.

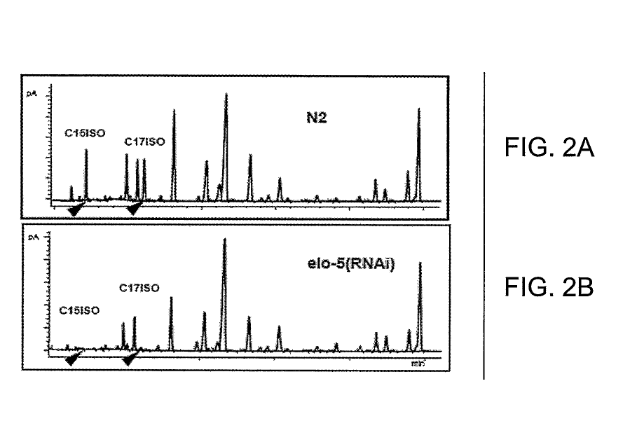

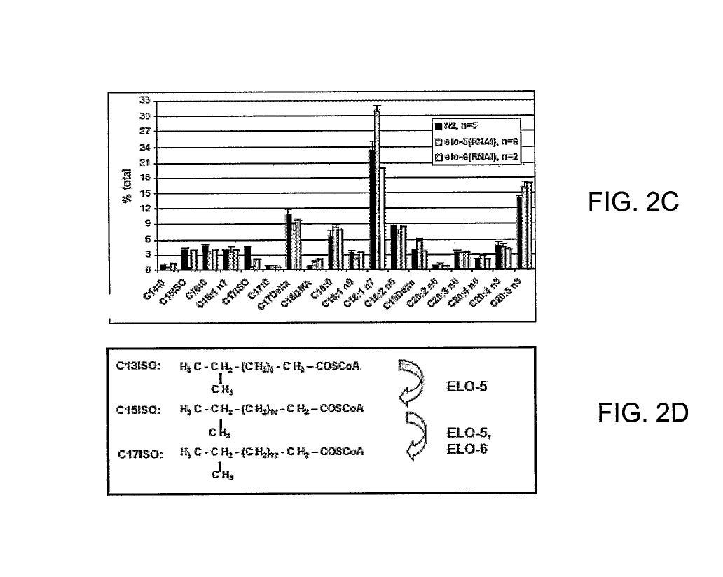

One component related to mmBCFA biosynthesis and function as described herein is the long chain fatty acid elongation enzyme, ELO-5, encoded by elo-S. In C. elegans (WormBase Sequence Name F41H10.7; WBGene00001243), the nucleic acid sequence encoding ELO-5 (elo-5) is represented herein by SEQ ID NO:9. SEQ ID NO:9 encodes the ELO-5 protein, the amino acid sequence of which is represented herein by SEQ ID NO:10. According to the present invention, ELO-5 and functional homologues thereof (which may also have structural homology to ELO-5) have the biological activity of being a fatty acid elongation enzyme that catalyzes the elongation reaction in the biosynthesis of C15ISO and C17ISO. The proposed enzymatic reaction is depicted in FIG. 2D. Structural homologues of ELO-5 have been identified in other organisms. While not necessarily orthologs, these homologues provide information about conserved structural regions in elongases. Such homologues include: Homo sapiens Elongation of very long chain fatty acids protein 3 (Accession No. ENSEMBL:ENSP00000238970); and Mus musculus Elongation of very long chain fatty acids protein 3 (Accession No. SW:O35949); all information in these accession numbers is incorporated herein by reference.

Another component related to mmBCFA biosynthesis and function as described herein is the long chain fatty acid elongation enzyme, ELO-6, encoded by elo-6. In C. elegans (WormBase Sequence Name F41H10.8; WBGene00001244), the nucleic acid sequence encoding ELO-6 (elo-6) is represented herein by SEQ ID NO:11. SEQ ID NO:11 encodes the ELO-6 protein, the amino acid sequence of which is represented herein by SEQ ID NO:12. According to the present invention, ELO-6 and functional homologues thereof (which may also have structural homology to ELO-6) have the biological activity of being a fatty acid elongation enzyme that catalyzes the elongation reaction in the biosynthesis of C17ISO. The proposed enzymatic reaction is depicted in FIG. 2D. Structural homologues of ELO-6 have been identified in other organisms. While not necessarily orthologs, these homologues provide information about conserved structural regions in elongases. Such homologues include: Homo sapiens Elongation of very long chain fatty acids protein 3 (Accession No. ENSEMBL:ENSP00000238970); and Mus musculus Elongation of very long chain fatty acids protein 3 (Accession No. SW:O35949); all information in these accession numbers is incorporated herein by reference.

Another component related to mmBCFA biosynthesis and function as described herein is the mmBCFA-specific acetyl-CoA synthetase, ACS-1 (also known as acetyl-CoA synthetase; acetyl activating enzyme; acetate thiokinase; acyl-activating enzyme; acetyl coenzyme A synthetase; acetic thiokinase; acetyl CoA ligase; acetyl CoA synthase; acetyl-coenzyme A synthase; short chain fatty acyl-CoA synthetase; short-chain acyl-coenzyme A synthetase; ACS), encoded by acs-1. In C. elegans (WormBase Sequence Name F46E10.1; WBGene00018488), the nucleic acid sequence encoding ACS-1 (asc-1) is represented herein by SEQ ID NO:13. SEQ ID NO:13 encodes the ACS-1 protein, the amino acid sequence of which is represented herein by SEQ ID NO:14. According to the present invention, ACS-1 and functional homologues thereof (which may also have structural homology to ACS-1) have the biological activity of catalyzing the synthesis of acetyl CoA (ATP+acetate+CoA=AMP+diphosphate+acetyl-CoA). In mmBCFA synthesis, this enzyme is required to activate the precursors for mmBCFA. Specifically, mmBCFA biosynthesis utilizes branched-chain α-keto-acids of leucine, isoleucine, and valine to produce mmBCFA acyl-CoA primers that substitute for acetyl-CoAs in the conventional FA biosynthesis. acs-1 expression is upregulated in mmBCFA deficient animals. Structural homologues of ACS-1 have been identified in other organisms. While not necessarily orthologs, these homologues provide information about conserved structural regions in acetyl CoA synthases. Such homologues include: Homo sapiens Hypothetical protein FLJ20920 (Accession No. ENSEMBL:ENSP00000300441); Bacillus subtilis Long-chain-fatty-acid-CoA ligase (Accession No. SW:P94547); all information in these accession numbers is incorporated herein by reference.

Another component related to mmBCFA biosynthesis and function as described herein is lipid depleted 1, LPD-1 (also known as sterol regulatory element binding protein (SREBP)), encoded by lpd-1. In C. elegans (WormBase Sequence Name Y47D3B.7; WBGene00004735), the nucleic acid sequence encoding LPD-1 (lpd-1) is represented herein by SEQ ID NO:15. SEQ ID NO:15 encodes the LPD-1 protein, the amino acid sequence of which is represented herein by SEQ ID NO:16. According to the present invention, LPD-1 and functional homologues thereof (which may also have structural homology to LPD-1) have the biological activity of being transcription factors that regulate the transcription of various genes involved in fatty acid biosynthesis and metabolism. LPD-1 has been shown to regulate the expression of several lipogenic enzymes, Acetyl-CoA Carboxilase (ACC), Fatty Acid synthetase (FAS) and Glycerol 3-Phosphate Acyltransferase (G3PA). ELO-5 and ELO-6 are believed to be targets of LPD-1 involved in mmBCFA biosynthesis. lpd-1 expression is upregulated in mmBCFA deficient animals. Structural and/or functional homologues of LPD-1 have been identified in other eukaryotic organisms. Such homologues include: Homo sapiens SREBP-1c (Accession No. NT_{—}010718 or AC122129); Rattus norvegicus SREBP-1 (Accession No. SW:P56720); all information in these accession numbers is incorporated herein by reference.

Another component related to mmBCFA biosynthesis and function as described herein is the long chain fatty acid elongation enzyme, nuclear hormone receptor-49 (NHR-49), encoded by nhr-49. In C. elegans (WormBase Sequence Name K10C3.6; WBGene00003639), the nucleic acid sequence encoding NHR-49 (nhr-49) is represented herein by SEQ ID NO:17. SEQ ID NO:17 encodes the NHR-49 protein, the amino acid sequence of which is represented herein by SEQ ID NO:18. According to the present invention, NHR-49 and functional homologues thereof (which may also have structural homology to NHR-49) have the biological activity of being regulators of fat usage, modulating pathways that control the consumption of fat and maintain a normal balance of fatty acid saturation. NHR-49 and homologues thereof can control the expression of other genes related to fatty acid and energy metabolism. Downregulation of NHR-49 results in up-regulation of saturated FA biosynthesis that may contribute to fat accumulation. nhr-49 expression is upregulated in mmBCFA deficient animals. Structural homologues of NHR-49 have been identified in other organisms. While not necessarily orthologs, these homologues provide information about conserved structural regions in nuclear hormone receptors. Such homologues include: Homo sapiens HNF4G protein (Accession No. ENSEMBL:ENSP00000346339); Mus musculus Hepatocyte nuclear factor 4-gamma (Accession No. SW:Q9WUU6); all information in these accession numbers is incorporated herein by reference.

Another component related to mmBCFA biosynthesis and function as described herein is RuvB-like DNA binding protein, RuvB-like, encoded by C27H6.2. In C. elegans (WormBase Sequence Name C27H6.2; WBGene00007784), the nucleic acid sequence encoding RuvB-like (protein name in C. elegans is C27H6.2) is represented herein by SEQ ID NO:19. SEQ ID NO:19 encodes the RuvB-like protein, the amino acid sequence of which is represented herein by SEQ ID NO:20. According to the present invention, RuvB-like and functional homologues thereof (which may also have structural homology to RuvB-like) have the biological activity of being a probable single-stranded DNA-stimulated ATPase and ATP-dependent DNA helicase (3′ to 5′). With particular regard to mmBCFA biosynthesis and function, the RuvB-like protein encoded by C27H6.2 affects the level of vaccenic acid (C18:1 n7), which is related to the levels of mmBCFA, suggesting cross talk between fatty acid biosynthesis pathways. Structural and/or functional homologues of RuvB-like DNA binding protein have been identified in other eukaryotic organisms. Such homologues include: human RuvB-like DNA binding protein-1 (Swiss-Prot. Accession No. Q9Y265 or NM_{—}003707); E. coli K12 (Accession No. NM_{—}003707); all information in these accession numbers is incorporated herein by reference.

Another component related to mmBCFA biosynthesis and function as described herein is pantothenate kinase, PNK-1, encoded by pnk-1. In C. elegans (WormBase Sequence Name C10G11.5; WBGene00004068), the nucleic acid sequence encoding PNK-1 (pnk-1) is represented herein by SEQ ID NO:21. SEQ ID NO:21 encodes the PNK-1 protein, the amino acid sequence of which is represented herein by SEQ ID NO:22. According to the present invention, PNK-1 and functional homologues thereof (which may also have structural homology to PNK-1) have the biological activity of catalyzing the conversion of CAATP and pantothenate to ADP and D-4′-phosphopantothenate, in the key regulatory step in the biosynthesis of coenzyme A (CoA). pnk-1 expression is upregulated in mmBCFA deficient animals and downregulation of pnk-1 expression downregulates mmBCFA expression. Structural and/or functional homologues of PNK-1 have been identified in other eukaryotic organisms. Such homologues include: Homo sapiens PNK-1, PNK-2, PNK-3 and PNK-4 (Accession Nos. gi55957270, gi55859625, gi62898131, and gi56204846, respectively); bacterial PNK (Accession Nos. gi23100137, gi49479594, gi42781996, gi52142613, and gi29896568); all information in these accession numbers is incorporated herein by reference.

Another component related to mmBCFA biosynthesis and function as described herein is branched-chain α-keto-acid dehydrogenase, BCKAD. In C. elegans (WormBase Sequence Name Y39E4A.3; WBGene00012713), the nucleic acid sequence encoding BCKAD α subunit is represented herein by SEQ ID NO:23. SEQ ID NO:23 encodes the BCKAD α subunit, the amino acid sequence of which is represented herein by SEQ ID NO:24. In C. elegans (WormBase Sequence Name T05H10.6; WBGene00011510), the nucleic acid sequence encoding BCKAD pyruvate dehydrogenase subunit is represented herein by SEQ ID NO:37. SEQ ID NO:27 encodes the BCKAD pyruvate dehydrogenase subunit, the amino acid sequence of which is represented herein by SEQ ID NO:38. According to the present invention, BCKAD and functional homologues thereof (which may also have structural homology to BCKAD) have the biological activity of catalyzing the overall conversion of alpha-keto acids to acyl-CoA and CO(2). The enzyme contains multiple copies of three enzymatic components: branched-chain alpha-keto acid decarboxylase (E1), lipoamide acyltransferase (E2) and lipoamide dehydrogenase (or pyruvate dehydrogenase) (E3) (also known as α-keto acid decarboxylase (E1, EC 1.2.4.4), dihydrolipoamide acyltransferase (E2, no EC number) and dihydrolipoamide reductase). BCKAD is a key enzyme in the synthesis of mmBCFA acyl-CoA primers. Structural and/or functional homologues of BCKAD α subunit have been identified in other eukaryotic organisms. Such homologues include: Homo sapiens BCKAD (Accession No. gi29391, gi62089242, gi5705948); bacterial BCKAD (Accession No. gi24373886, gi56460781); all information in these accession numbers is incorporated herein by reference. Structural and/or functional homologues of BCKAD pyruvate dehydrogenase subunit have been identified in other eukaryotic organisms. Such homologues include: Homo sapiens BCKAD (Accession No. gi57209621, gi387011, gi33357461); bacterial BCKAD (Accession No. gi23347958, gi62290042, gi45917129, gi/5074378); all information in these accession numbers is incorporated herein by reference.

Another component related to mmBCFA biosynthesis and function as described herein is oligopeptide transporter, PEP-2, encoded by pep-2. In C. elegans (WormBase Sequence Name K04E7.2; WBGene00003877), the nucleic acid sequence encoding PEP-2 (pep-2) is represented herein by SEQ ID NO:25. SEQ ID NO:25 encodes the PEP-2 protein, the amino acid sequence of which is represented herein by SEQ ID NO:26. According to the present invention, PEP-2 and functional homologues thereof (which may also have structural homology to PEP-2) have the biological activity of an oligopeptide transporter for uptake of di-/tripeptides. Structural and/or functional homologues of PEP-2 have been identified in other eukaryotic organisms. Such homologues include: Homo sapiens PEP-2 (Accession Nos. gi2832268, gi2833272, gi33126130); bacterial PEP-2 (Accession Nos. gi66856385, gi24371602, gi48853934, gi21230862, gi21107622, gi32448003, gi53803599, gi66856386, gi53681929, gi58581973, gi34102472, gi/6802598, gi47094789, gi46906800); all information in these accession numbers is incorporated herein by reference.

Another component related to mmBCFA biosynthesis and function as described herein is phosphoinositide-dependent protein kinase 1, PDK-1, encoded by pdk-1. In C. elegans (WormBase Sequence Name H42K12.1; WBGene00003965), the nucleic acid sequence encoding PDK-1 (pdk-1) is represented herein by SEQ ID NO:27. SEQ ID NO:27 encodes the PDK-1 protein, the amino acid sequence of which is represented herein by SEQ ID NO:28. According to the present invention, PDK-1 and functional homologues thereof (which may also have structural homology to PDK-1) have the biological activity of protein serine/threonine kinase activity. Structural homologues of NHR-49 have been identified in other organisms. While not necessarily orthologs, these homologues provide information about conserved structural regions in phosphoinositide-dependent protein kinases. Such homologues include: human 3-phosphoinositide dependent kinase 1 (Accession No. ENSEMBL:ENSP00000344220); Mus musculus 3-phosphoinositide dependent protein kinase-1 (Accession No. SW:Q9Z2A0); all information in these accession numbers is incorporated herein by reference.

Another component related to mmBCFA function as described herein is the insulin-like receptor, DAF-2, encoded by daf-2. In C. elegans (WormBase Sequence Name Y55D5A.5; WBGene00000898), the nucleic acid sequence encoding DAF-2 (daf-2) is represented herein by SEQ ID NO:29. SEQ ID NO:29 encodes the DAF-2 protein, the amino acid sequence of which is represented herein by SEQ ID NO:30. According to the present invention, DAF-2 and functional homologues thereof (which may also have structural homology to DAF-2) have the biological activity of being a receptor tyrosine kinase, that binds to insulin, as well as other ligands (DAF-28, INS-1, INS-7). Structural homologues of DAF-2 have been identified in other organisms. While not necessarily orthologs, these homologues provide information about conserved structural regions in insulin receptor-like proteins. Such homologues include: Homo sapiens insulin receptor precursor (Accession No. ENSEMBL:ENSP00000342838); all information in these accession numbers is incorporated herein by reference.

Another component that can be used to evaluate mmBCFA function as described herein is the kinesin-like protein, Zygotic epidermal Enclosure defective (ZEN-4), encoded by zen-4. In C. elegans (WormBase Sequence Name M03D4.1; WBGene00006974), the nucleic acid sequence encoding ZEN-4 (zen-4) is represented herein by SEQ ID NO:31. SEQ ID NO:31 encodes the ZEN-4 protein, the amino acid sequence of which is represented herein by SEQ ID NO:32. According to the present invention, ZEN-4 and functional homologues thereof (which may also have structural homology to ZEN-4) have the biological activity of being a kinesin-like protein associated with microtubule-based movement, and has ATP binding microtubule motor activity. Structural homologues of ZEN-4 have been identified in other organisms. While not necessarily orthologs, these homologues provide information about conserved structural regions in kinesin-like proteins. Such homologues include: Homo sapiens Kinesin family member 23 isoform 1 (Accession No. ENSEMBL:ENSP00000260363); Mus musculus Kinesin family member 20A (Accession No. SW:P97329); all information in these accession numbers is incorporated herein by reference.

Another component that can be used to evaluate mmBCFA function as described herein is polarity and osmotic sensitivity defect protein, POD-1, encoded by pod-1. In C. elegans (WormBase Sequence Name Y76A2B.1; WBGene00004075), the nucleic acid sequence encoding POD-1 (pod-1) is represented herein by SEQ ID NO:33. SEQ ID NO:33 encodes the POD-1 protein, the amino acid sequence of which is represented herein by SEQ ID NO:34. According to the present invention, POD-1 and functional homologues thereof (which may also have structural homology to POD-1) have the biological activity of being a coronin-like protein required for asymmetry along the anterior-posterior axis at the beginning of embryonic development. Structural homologues of POD-1 have been identified in other organisms. While not necessarily orthologs, these homologues provide information about conserved structural regions in coronin-like proteins. Such homologues include: Homo sapiens Coronin 7 (Accession No. ENSEMBL:ENSP00000251166); Mus musculus Coronin 7 (Accession No. SW:Q9D2B7); all information in these accession numbers is incorporated herein by reference.

Another component that can be used to evaluate mmBCFA function as described herein is the superoxide dismutase, SOD-3, encoded by sod-3. In C. elegans (WormBase Sequence Name C08A9.1; WBGene00004932), the nucleic acid sequence encoding SOD-3 (sod-3) is represented herein by SEQ ID NO:35. SEQ ID NO:35 encodes the SOD-3 protein, the amino acid sequence of which is represented herein by SEQ ID NO:36. According to the present invention, SOD-3 and functional homologues thereof (which may also have structural homology to SOD-3) have the biological activity of being an iron/manganese superoxide dismutase. Structural homologues of SOD-3 have been identified in other organisms. While not necessarily orthologs, these homologues provide information about conserved structural regions in superoxide dismutases. Such homologues include: Homo sapiens superoxide dismutase (Accession No. ENSEMBL:ENSP00000337127); Rattus norvegicus superoxide dismutase (Accession No. SW:P07895); all information in these accession numbers is incorporated herein by reference.

In any of the methods or compositions described herein, one can use a full-length gene, including a regulatory region of the gene, or a nucleic acid molecule encoding the gene product (protein encoded by the gene) or any fragment of such nucleic acid molecules, or any gene product (i.e., encoded protein or peptide) or fragment thereof that is suitable for use in a method to identify regulators of the target for the purpose of regulating mmBCFA biosynthesis and/or homeostasis and/or growth, development and/or reproduction of an organism.

In one embodiment of the invention, the regulation of the concentration or activity of a target gene or product by a regulatory compound induces, enhances, upregulates or otherwise increases the expression or activity of a cellular component required for the biosynthesis or function of mmBCFA and its derivatives in an organism. In another embodiment, the regulation of the concentration or activity of a target gene or product by a regulatory compound depletes, inhibits, reduces or otherwise downregulates the expression or activity of a cellular component that normally inhibits the biosynthesis or function of mmBCFA and its derivatives in an organism, such that the biosynthesis or function of mmBCFA and its derivatives is increased or induced. In one embodiment, two genes are members of the same mmBCFA biological pathway and one gene or gene product regulates the expression or activity of the other gene or gene product. In another preferred embodiment of the invention, two genes are members of the same mmBCFA biological pathway and the substrate of a protein encoded by one gene is a product of a biochemical reaction mediated by the protein encoded by the other gene. In one embodiment, at least one of the target genes encodes an enzyme.

Target genes or proteins identified according to the present invention can be evaluated using a variety of methods to validate their involvement in metabolism and homeostasis, cell growth, development and/or reproduction or any other biological process related to mmBCFA biosynthesis and function. Such methods include methods that disrupt or “knock out” the expression of a target gene and/or its encoded product in a cell or organism. Knock-out methods include somatic cell knock-outs and inhibitory RNA molecules including anti-sense oligonucleotides, siRNA molecules, RNAi molecules (described herein), and RNA decoys, as well as methods using other transposable elements and even antibodies. Target genes or proteins can also be evaluated by methods that include nucleic acid-based experiments such as Northern Blots, Real Time polymerase chain reaction or high density microarrays.

Once one or more members of a biological pathway are identified as required for mmBCFA biosynthesis and/or function (such functions including, but not limited to, metabolism and homeostasis, growth, development or reproductive functions), the present invention can include identifying additional members of mmBCFA biological pathways that are also required for these functions and activities. Such subsequent identification is within the skill of one in the art and can be achieved, for example, using the model systems of the invention.

It will be understood that this invention is not limited to the particular methodology, protocols, cell lines, animal species or genera, constructs, or reagents described herein, as such may vary. It is also to be understood that the terminology used herein is for the purpose of describing particular embodiments only, and is not intended to limit the scope of the invention that will be limited only by the appended claims. All technical and scientific terms used herein have the same meaning as commonly understood to one of ordinary skill in the art to which this invention belongs unless clearly indicated otherwise.

As discussed above, one embodiment of the present invention relates to methods for identifying pharmaceutical and/or nutraceutical or dietary compounds (including any therapeutic compounds) that regulate mmBCFA biosynthesis and/or function (including metabolism and/or homeostasis and/or related metabolism (including food signaling processes), growth, development and/or reproduction of an organism or cell by regulating genes or gene products involved in the control of these biological activities and functions. Once a gene or protein has been identified as a target useful in the present invention, an assay can be used for screening and selecting a chemical compound, a nucleic acid compound, or a biological compound having a regulatory activity that is useful in the regulation of mmBCFA and its derivatives related biological processes in an organism. Reference herein to inhibiting a target, can refer to one or both of inhibiting expression of a target gene and inhibiting the translation and/or activity of its corresponding expression product. Similarly, reference herein to inducing or enhancing a target, can refer to one or both of inducing or enhancing the expression of a target gene and inducing or enhancing the translation and/or activity of its corresponding expression product.

In one embodiment, an organism or cell that naturally expresses the gene of interest or has been transfected with the gene or other recombinant nucleic acid molecule encoding the protein of interest is contacted or incubated with various compounds, also referred to as candidate compounds, test compounds, or putative regulatory compounds. Regulation of the target gene or target protein, or regulation of activities associated with the target gene or target protein, are then evaluated. Putative therapeutic compounds identified in this manner can then be re-tested, if desired, in other assays to confirm their activities in the mmBCFA biological processes.

In general, the biological activity or biological action of a protein or lipid (including fatty acids) refers to any function(s) exhibited or performed by the protein or lipid that is ascribed to the naturally occurring form of the protein or lipid as measured or observed in vivo (i.e., in the natural physiological environment of the protein or lipid) or in vitro (i.e., under laboratory conditions). As used herein the term “lipids” will refer generally to a variety of lipids, such as phospholipids; free fatty acids; esters of fatty acids; triacylglycerols; diacylglycerides; monoacylglycerides; lysophospholipids; phosphatides; sterols and sterol esters; hydrocarbons; pigments and other lipids, and lipid associated compounds. For the sake of brevity, unless otherwise stated, the term “lipid” refers to lipid and/or lipid-associated compounds.

Modifications, activities or interactions which result in a decrease in protein expression or lipid biosynthesis, or a decrease in the activity of the protein or lipid, can be referred to as inactivation (complete or partial), down-regulation, reduced action, or decreased action or activity of a protein or lipid. Similarly, modifications, activities or interactions which result in an increase in protein expression or lipid biosynthesis, or an increase in the activity of the protein or lipid, can be referred to as amplification, overproduction, activation, enhancement, up-regulation or increased action of a protein or lipid. The biological activity of a protein or lipid according to the invention can be measured or evaluated using any assay for the biological activity of the protein or lipid as known in the art. For proteins, such assays can include, but are not limited to, binding assays, two hybrid systems, assays to determine internalization of the protein and/or associated proteins, enzyme assays, cell signal transduction assays (e.g., phosphorylation assays), and/or assays for determining downstream cellular events that result from activation or binding of the protein (e.g., expression of downstream genes, production of various biological mediators, etc.). For lipids, such assays can include binding assays, two hybrid systems, and/or assays for determining downstream cellular events that result from production of the lipids or association of the lipids with particular biological mediators. Many such activities are described herein.

According to the present invention, a biologically active fragment or homologue (defined more specifically below) of a gene or protein maintains the ability to be useful in a method of the present invention. Therefore, the biologically active fragment or homologue maintains the ability to be used to identify regulators of a target when, for example, the biologically active fragment or homologue is expressed by a cell or organism. Therefore, the biologically active fragment or homologue has a structure that is sufficiently similar to the structure of the native gene or protein that a regulatory compound can be identified by its ability to bind to and/or regulate the expression or activity of the fragment or homologue in a manner consistent with the regulation of the native gene or protein.

In another embodiment, a modified non-human organism, with or without additional dietary supplementation, is contacted with or otherwise administered (e.g., by feeding or injection) a putative regulatory compound, and a change in the non-human organism is evaluated in the presence and absence of the putative regulatory compound. The non-human organism can be modified by any method described herein, which includes modification at the gene level, the RNA level, the protein level, or combinations thereof. Preferably, the organism has a modification that results in the deletion or inactivation of at least one protein (i.e., two, three, four or more proteins can be deleted or inactivated) involved in mmBCFA biosynthesis and/or function, such proteins including, but not limited to: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (RuvB-like) (SEQ ID NO:20), pantothenate kinase (PNK-1) (SEQ ID NO:22), branched-chain α-keto-acid dehydrogenase (BCKAD) (SEQ ID NO:24 (α subunit) and SEQ ID NO:38 (pyruvate dehydrogenase subunit)), oligopeptide transporter PEP-2 (PEP-2) (SEQ ID NO:26); phosphoinositide-dependent protein kinase 1(PDK-1) (SEQ ID NO:28); and insulin receptor DAF-2 (DAF-2) (SEQ ID NO:30).

In this embodiment, a change to be detected in the organism can be any change that is indicative of a difference in the biosynthesis or function of mmBCFA in the presence of the putative regulatory compound as compared to the absence of the compound and can include, but is not limited to, (i) an increase or decrease in the expression or biological activity of the protein or homologue thereof that has been modified; (ii) an increase or decrease in the amount or type of mmBCFA synthesized by the non-human animal; (iii) a change in the total fatty acid profile of the non-human animal; (iv) an increase or decrease in insulin-signaling in the non-human animal; (v) a change in embryogenesis in the non-human animal or progeny thereof; (vi) a change in the fertility of the non-human animal or progeny thereof; (vii) a change in the viability of progeny of the non-human animal; (viii) an increase or decrease in the growth or development of the non-human animal or progeny thereof; and (ix) a change in a metabolic response to food sensation in the non-human animal. Methods for measuring each of these activities is exemplified in the Examples for C. elegans and will be known to those of skill in the art.

Compounds to be screened in the methods of the invention include known organic compounds such as antibodies, products of peptide libraries, and products of chemical combinatorial libraries. Compounds may also be identified using rational drug design relying on the structure of the product of a gene. Such methods are known to those of skill in the art and involve the use of three-dimensional imaging software programs. For example, various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

As used herein, a mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art.

A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

As used herein, the term “test compound”, “putative inhibitory compound” or “putative regulatory compound” refers to compounds having an unknown or previously unappreciated regulatory activity in a particular process. As such, the term “identify” with regard to methods to identify compounds is intended to include all compounds, the usefulness of which as a regulatory compound for the purposes of regulating a biological process associated with mmBCFA is determined by a method of the present invention.

In one embodiment of the invention, regulatory compounds are identified by exposing a target gene to a test compound; measuring the expression of a target; and selecting a compound that regulates (up or down) the expression or activity of the target. For example, the putative regulator can be exposed to a cell that expresses the target (endogenously or recombinantly).

The conditions under which an organism, a cell, a cell lysate, a nucleic acid molecule or a protein is exposed to or contacted with a putative regulatory compound, such as by mixing, combining or plating, are any suitable culture or assay conditions. The Examples section herein and PCT Publication WO 00/76308, supra, describe assays for testing putative regulatory compounds and reagents in a “worm assay” or an assay system using C. elegans. The C. elegans system described in the present invention is particularly useful for screening compounds that regulate the mmBCFA biosynthetic and metabolic pathway due to the provision of significant detail regarding components of the system and the biological effects of mmBCFA in eukaryotes by the present inventors.

In the case of a cell-based assay, the conditions include an effective medium in which the cell can be cultured or in which the cell lysate can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested. Suitable conditions for contacting an organism with a particular compound are exemplified in the Examples section, which describe methods for exposing the organism C. elegans to mmBCFA and to RNAi (e.g., by feeding). Other conditions may include injection or topical administration or other administration routes (described below).

As used herein, the term “expression”, when used in connection with detecting the expression of a target of the present invention, can refer to detecting transcription of the target gene and/or to detecting translation of the target protein encoded by the target gene. To detect expression of a target refers to the act of actively determining whether a target is expressed or not. This can include determining whether the target expression is upregulated as compared to a control, downregulated as compared to a control, or unchanged as compared to a control. Therefore, the step of detecting expression does not require that expression of the target actually is upregulated or downregulated, but rather, can also include detecting that the expression of the target has not changed (i.e., detecting no expression of the target or no change in expression of the target). Expression of transcripts and/or proteins is measured by any of a variety of known methods in the art. For RNA expression, methods include but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

Yet another embodiment of the present invention relates to methods to identify additional genes, proteins, or other moieties (e.g., other lipids or fatty acids) that are associated with the mmBCFA biosynthetic process and physiological processes related thereto. For example, the present inventors have demonstrated herein the use of the mmBCFA system of the invention to identify multiple genes whose regulation is associated with the regulation of mmBCFA (see Examples). Such methods and the genes and encoded products identified thereby are all encompassed by the present invention. In addition, the model animal and cell systems described herein can be manipulated using any of a variety of genetic and other techniques to further evaluate components of mmBCFA biosynthesis and function and the effects of various treatments on the system.

Another embodiment of the present invention relates to compositions and methods for regulating the metabolism and/or homeostasis of mmBCFA and its derivatives in an organism, for regulating the growth, development and/or reproduction of an organism, or for regulating food sensing and insulin signaling in the organism. In one aspect, such a method includes administering (e.g., by feeding or other suitable means) an amount of at least one mmBCFA (including an mmBCFA a carbon chain length of at least 13 carbons, and more preferably at least 15 carbons, and more preferably at least 17 carbons or more), and derivatives thereof, or a composition (formulation) comprising the same, sufficient to regulate the metabolism and/or homeostasis of mmBCFA in an organism, and/or to regulate the growth, development and/or reproduction of an organism. For example, suitable mmBCFA include, but are not limited to, long chain mmBCFA or precursors thereof, including C13ISO, C15ISO, C17ISO, C15ante-ISO, C17-anteISO and/or any derivative thereof, including methyl esters of any of these mmBCFAs. According to the present invention, a long chain mmBCFA is a mmBCFA having a carbon chain length of at least 15 carbons. Preferred long chain mmBCFA for use in the invention are the C15 and C17 forms. In addition, the C13ISO form, being a precursor for the C15 and C17 forms, can be used in the invention, as discussed in detail herein.

In another embodiment, the method includes regulating the biosynthesis and/or function of endogenous mmBCFA in an organism by modifying the organism or cells thereof (e.g., by genetic or other modification described herein, including by upregulation or downregulation or overexpression of a gene or protein associated with mmBCFA biosynthesis or function) to regulate such biosynthesis and/or function and/or by administering to the organism a compound or formulation that regulates such biosynthesis and/or function. These aspects of the invention can be used to regulate various biological processes in the organism that the present inventors have shown are associated with mmBCFA including, but not limited to, metabolism, homeostasis, growth, development and reproduction. In addition, compositions of the invention may be used as novel compositions to control the ratios and compositions of different fatty acids in an organism, including fatty acids other than mmBCFA. With regard to the latter, a use of gas chromatography analysis of FA composition in total lipids obtained from a whole organism or individual tissues and cells is suggested to monitor changes in FA homeostasis. This method could be used, for example, for confirmation in a screen for compounds that compensate for the mmBCFA deficiency. Alternatively, one could modify the total fatty acid profile in an organism by manipulating (up or down) the mmBCFA in the organism as described herein.

In one embodiment of the invention, a pharmaceutical, nutraceutical or dietary composition (formulation) is prepared from an effective amount of a regulatory agent or a mmBCFA of the invention and a pharmaceutically-acceptable carrier. According to the present invention, a pharmaceutical formulation typically refers to a formulation used for a medical purpose, such as to treat, prevent or ameliorate a disease or condition or a symptom thereof. A nutraceutical formulation is typically a formulation that is a combination of nutritional (or dietary) and pharmaceutical product and is intended to be used to provide enhanced health benefits to an individual. Regulatory processes are typically more strict for pharmaceutical formulations than for nutraceutical formulations. Dietary formulations are more typically considered to be any health-enhancing or health-maintaining product derived from nature and would typically be used to supplement the diet of an individual to provide a positive health benefit, and might help prevent a disease or condition, but is not necessarily intended to treat a disease or condition. However, it is to be understood that a pharmaceutical, nutraceutical and dietary composition/formulation may be identical to one another, with the designation depending on the intended use of the formulation and/or other compounds that are to be administered or used with the formulation.

Pharmaceutically-acceptable carriers are well known to those with skill in the art and can be used in any formulation described herein, including pharmaceutical, nutraceutical and dietary formulations. The compositions/formulations of the present invention can be manufactured in a manner that is itself known, e.g., by means of a conventional mixing, dissolving, granulating, dragee-making, levigating, emulsifying, encapsulating, entrapping or lyophilizing processes.

Compositions for use in accordance with the present invention thus can be formulated in conventional manner using one or more physiologically or pharmaceutically acceptable carriers comprising excipients and auxiliaries which facilitate processing of the active compounds into preparations which can be used pharmaceutically. Proper formulation is dependent upon the route of administration chosen and the intended use of the composition. According to the present invention, a pharmaceutically acceptable carrier includes pharmaceutically acceptable excipients and/or pharmaceutically acceptable delivery vehicles, which are suitable for use in administration of the composition to a suitable in vitro, ex vivo or in vivo site. Preferred pharmaceutically acceptable carriers are capable of maintaining a compound, a lipid, a protein, a peptide, nucleic acid molecule or mimetic (drug) according to the present invention in a form that, upon arrival of the compound, lipid, protein, peptide, nucleic acid molecule or mimetic at the desired site in a culture or organism, the compound, lipid, protein, peptide, nucleic acid molecule or mimetic is capable of interacting with its target. Suitable excipients of the present invention include excipients or formularies that transport or help transport, but do not specifically target a composition to a cell or into an organism (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

For injection, the compounds of the invention can be formulated in appropriate aqueous solutions, such as physiologically compatible buffers such as Hanks's solution, Ringer's solution, or physiological saline buffer. For transmucosal and transcutaneous administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as foods and food products, tablets, pills, dragees, capsules, liquids, gels, syrups, slurries, suspensions and the like, for oral ingestion by a patient to be treated. Suitable food products include, but are not limited to, fine bakery wares, bread and rolls, breakfast cereals, processed and unprocessed cheese, condiments (ketchup, mayonnaise, etc.), dairy products (milk, yogurt), puddings and gelatin desserts, carbonated drinks, teas, powdered beverage mixes, processed fish products, fruit-based drinks, chewing gum, hard confectionery, frozen dairy products, processed meat products, nut and nut-based spreads, pasta, processed poultry products, gravies and sauces, potato chips and other chips or crisps, chocolate and other confectionery, soups and soup mixes, soya based products (milks, drinks, creams, whiteners), vegetable oil-based spreads, and vegetable-based drinks.

The compounds and compositions of the present invention, including mmBCFAs as discussed herein, can be administered to a patient or organism alone or in combination with pharmaceutically acceptable carriers, as noted above, the proportion of which is determined by the solubility and chemical nature of the compound, chosen route of administration and standard pharmaceutical practice.

The compounds and compositions of the present invention can be administered to a patient to achieve a desired physiological effect. Preferably the patient is an animal, more preferably a mammal, and most preferably a human. The compound can be administered in a variety of forms adapted to the chosen route of administration, e.g., orally or parenterally. Parenteral administration in this respect includes, but is not limited to, administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

In the method of the present invention, a compound, or compositions comprising such compounds, can be administered to any organism, and particularly, to any eukaryote, and more particularly to any invertebrate or vertebrate, and even more particularly, to any member of the vertebrate class, Mammalia, including, without limitation, primates, rodents, livestock and domestic pets. Typically, it is desirable to obtain a therapeutic or nutritional benefit in a patient. A therapeutic benefit is not necessarily a cure for a particular disease or condition, but rather, preferably encompasses a result which can include alleviation of the disease or condition, elimination of the disease or condition, reduction of a symptom associated with the disease or condition, prevention or alleviation of a secondary disease or condition resulting from the occurrence of a primary disease or condition, and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment) to reduce the symptoms of the disease. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

In one embodiment of the present invention, a long chain MMBCFA or precursor thereof is administered to a patient that has Maple Syrup Urine Disease (MSUD). MSUD is caused by the inability to metabolize the branched-chain amino acids: leucine, isoleucine, and valine. Urine from these patients has an odor that is reminiscent of maple syrup or burnt sugar, thus the name. Untreated, MUSD causes ketoacidosis, neurological damage (e.g., mental retardation) and death. Conventional treatments for MUSD include the strict use of a special diet that contains very low levels of the amino acids leucine, isoleucine, and valine to avoid the accumulation of these amino acids in the body of the patient. Since the branched-chain amino acids are precursors of the mmBCFA that are now shown herein to be important to a diverse array of physiological functions, such functions may be impaired in patients having restricted branched chain amino acid intake. Therefore, the present invention provides for the dietary supplementation of patients with MUSD with long chain mmBCFA or precursors thereof, including C13ISO, C15ISO, C17ISO, C15ante-ISO, C17-anteISO and/or any derivative thereof, including methyl esters of any of these mmBCFAs. The long chain mmBCFA useful in this invention can be provided essentially alone (e.g., as a mmBCFA fatty acid supplement, which may include a suitable pharmaceutically acceptable carrier), or in combination with other pharmaceutical (e.g., agents for the treatment of MSUD, or for a symptom thereof) and/or nutraceutical or dietary agents (e.g., vitamins, minerals, prescribed limited quantities of branched-chain amino acids, proteins and other agents).

Another embodiment of the present invention relates to a dietary supplement comprising an amount of at least one mmBCFA sufficient to regulate the metabolism and/or homeostasis of mmBCFA in an organism, and/or to regulate the metabolism (including food signaling processes), growth, development and/or reproduction of an organism. In one aspect of the invention, the mmBCFA is selected from C13ISO, C15ISO, C17ISO, C15ante-ISO, C17-anteISO and/or any derivative thereof, including methyl esters of any of these mmBCFAs.

In one aspect of the invention, any of the above-described dietary supplements or pharmaceutical and nutraceutical compositions may contain one or more additional components that are useful for the particular application of the composition. For example, a dietary supplement may contain vitamins, minerals, proteins, and/or additional fatty acids that may be of benefit to the patient. A pharmaceutical composition may contain additional drugs or compounds that are useful for treating or preventing a condition related to growth, development and/or reproduction or any other aspect of mmBCFA biological processes.

The following are various additional definitions and descriptions of aspects of the invention described above or useful in the present invention as described herein.

An isolated protein, according to the present invention, is a protein (including a peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. An isolated protein useful according to the present invention can be isolated from its natural source, produced recombinantly or produced synthetically. Smaller peptides useful as regulatory peptides are typically produced synthetically by methods well known to those of skill in the art.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein. A functional homologue is a homologue of a reference protein that may have any degree of structural similarity to the reference protein and has the same or essentially the same function as the reference protein. Typically, a functional homologue is structurally similar to the reference protein at least at conserved regions of the protein that are required for the function of the protein (e.g., catalytic domain, substrate binding site, cofactor binding site, DNA binding site, receptor or ligand binding site, signal transduction domains). An ortholog is an example of a functional homologue. Therefore, reference to a homologue can include an ortholog. An ortholog is a gene in two or more species that has evolved from a common ancestor and therefore has a common function. An ortholog is also called an orthologous gene.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

An agonist, as used herein, is a compound that is characterized by the ability to agonize (e.g., stimulate, induce, increase, enhance, or mimic) the biological activity of a naturally occurring or reference protein or compound. More particularly, an agonist can include, but is not limited to, a compound, protein, peptide, or nucleic acid that mimics or enhances the activity of the natural or reference compound, and includes any homologue, mimetic, or any suitable product of drug/compound/peptide design or selection which is characterized by its ability to agonize (e.g., stimulate, induce, increase, enhance) the biological activity of a naturally occurring or reference compound.

An antagonist refers to any compound which inhibits (e.g., antagonizes, reduces, decreases, blocks, reverses, or alters) the effect of a naturally occurring or reference compound as described above. More particularly, an antagonist is capable of acting in a manner relative to the activity of the reference compound, such that the biological activity of the natural or reference compound, is decreased in a manner that is antagonistic (e.g., against, a reversal of, contrary to) to the natural action of the reference compound. Such antagonists can include, but are not limited to, any compound, protein, peptide, or nucleic acid (including ribozymes and antisense) or product of drug/compound/peptide design or selection that provides the antagonistic effect.

Agonists and antagonists that are products of drug design can be produced using various methods known in the art. Various methods of drug design, useful to design mimetics or other compounds useful in the present invention are disclosed in Maulik et al., 1997, supra.

According to the invention, reference to an “isolated nucleic acid molecule” refers to a nucleic acid molecule that is the size of or is smaller than a gene. Thus, an isolated nucleic acid molecule does not encompass isolated total genomic DNA or an isolated chromosome. As used herein, the term “gene” has the meaning that is well known in the art, that is, a nucleic acid sequence that includes the translated sequences that code for a protein (“exons”) and the untranslated intervening sequences (“introns”), and any regulatory elements necessary to transcribe and/or translate the protein. Included in the invention are nucleic acid molecules that are less than a full-length gene or less than a full-length coding sequence, such as fragments of a gene or coding sequence comprising, consisting essentially of, or consisting of, for example, a fragment of any of the nucleic acid sequences for target genes described in the present invention. A coding sequence can include genomic DNA without introns, cDNA or RNA that encodes a protein. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., are heterologous sequences).

In one embodiment, an isolated nucleic acid molecule useful in a method of the present invention is produced using recombinant DNA technology (e.g., polymerase chain reaction (PCR) amplification, cloning) or chemical synthesis. A nucleic acid molecule homologue can be produced using a number of methods known to those skilled in the art (see, for example, Sambrook et al., ibid.). For example, nucleic acid molecules can be modified using a variety of techniques including, but not limited to, classical mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

The term isolated nucleic acid molecule does not necessarily connote any specific minimum length unless set forth by reference to a minimum number of nucleotides or by a function of the nucleic acid molecule. The minimum size of a nucleic acid molecule of the present invention is generally a size sufficient to encode a protein having the desired biological activity, a size sufficient to inhibit the expression and/or activity of a target as described herein, a size sufficient for use in a screening assay of the invention, or a size sufficient to form a probe or oligonucleotide primer that is capable of forming a stable hybrid with the complementary sequence of a nucleic acid molecule. As such, the size of a nucleic acid molecule of the present invention can be dependent on nucleic acid composition and percent homology or identity between the nucleic acid molecule and complementary sequence as well as upon hybridization conditions per se (e.g., temperature, salt concentration, and formamide concentration) and the intended use of the nucleic acid molecule. The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a fragment of a gene, a portion of a protein encoding sequence, or a nucleic acid sequence encoding a full-length protein (including a complete gene).

Some embodiments of the present invention may include the production and/or use of a recombinant nucleic acid molecule comprising a recombinant vector and a nucleic acid molecule comprising a nucleic acid sequence encoding a gene or fragment thereof as described herein. According to the present invention, a recombinant vector is an engineered (i.e., artificially produced) nucleic acid molecule that is used as a tool for manipulating a nucleic acid sequence of choice and for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid molecules of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant organism (e.g., a microbe or a plant). The entire vector can remain in place within a host cell, or under certain conditions, the plasmid DNA can be deleted, leaving behind the nucleic acid molecule of the present invention. The integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced is inserted into the recombinant vector to produce a recombinant nucleic acid molecule. The nucleic acid sequence encoding the protein to be produced is inserted into the vector in a manner that operatively links the nucleic acid sequence to regulatory sequences in the vector that enable the transcription and translation of the nucleic acid sequence within the recombinant host cell.

In another embodiment, a recombinant vector used in a recombinant nucleic acid molecule of the present invention is a targeting vector. As used herein, the phrase “targeting vector” is used to refer to a vector that is used to deliver a particular nucleic acid molecule into a recombinant host cell, wherein the nucleic acid molecule is used to delete or inactivate an endogenous gene within the host cell or microorganism (i.e., used for targeted gene disruption or knock-out technology). Such a vector may also be known in the art as a “knock-out” vector. In one aspect of this embodiment, a portion of the vector, but more typically, the nucleic acid molecule inserted into the vector (i.e., the insert), has a nucleic acid sequence that is homologous to a nucleic acid sequence of a target gene in the host cell (i.e., a gene which is targeted to be deleted or inactivated). The nucleic acid sequence of the vector insert is designed to bind to the target gene such that the target gene and the insert undergo homologous recombination, whereby the endogenous target gene is deleted, inactivated or attenuated (i.e., by at least a portion of the endogenous target gene being mutated or deleted).

Typically, a recombinant nucleic acid molecule includes at least one nucleic acid molecule of the present invention operatively linked to one or more expression control sequences, including transcription control sequences and translation control sequences. As used herein, the phrase “recombinant molecule” or “recombinant nucleic acid molecule” primarily refers to a nucleic acid molecule or nucleic acid sequence operatively linked to an expression control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence (e.g., a transcription control sequence and/or a translation control sequence) in a manner such that the molecule is expressed when transfected (i.e., transformed, transduced, transfected, conjugated or conduced) into a host cell. Transcription control sequences are sequences that control the initiation, elongation, or termination of transcription. Particularly important transcription control sequences are those that control transcription initiation, such as promoter, enhancer, operator and repressor sequences. Suitable transcription control sequences include any transcription control sequence that can function in a host cell or organism into which the recombinant nucleic acid molecule is to be introduced.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells. In microbial systems, the term “transformation” is used to describe an inherited change due to the acquisition of exogenous nucleic acids by the microorganism and is essentially synonymous with the term “transfection.” However, in animal cells, transformation has acquired a second meaning that can refer to changes in the growth properties of cells in culture (described above) after they become cancerous, for example. Therefore, to avoid confusion, the term “transfection” is preferably used with regard to the introduction of exogenous nucleic acids into animal cells, including human cells, and is used herein to generally encompass transfection of animal cells and transformation of microbial cells, to the extent that the terms pertain to the introduction of exogenous nucleic acids into a cell. Therefore, transfection techniques include, but are not limited to, transformation, chemical treatment of cells, particle bombardment, electroporation, microinjection, lipofection, adsorption, infection and protoplast fusion.

A recombinant cell is preferably produced by transforming a host cell with one or more recombinant molecules, each comprising one or more nucleic acid molecules operatively linked to an expression vector containing one or more expression control sequences.

“Hybridization” has the meaning that is well known in the art, that is, the formation of a duplex structure by two single-stranded nucleic acids due to complementary base pairing. Hybridization can occur between exactly complementary nucleic acid strands or between nucleic acid strands that contain some regions of mismatch. As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety. “Stringent hybridization” has a meaning well-established in the art, that is, hybridization performed at a salt concentration of no more than 1M and a temperature of at least 25 degrees Celsius. For example, conditions of 5×SSPE (750 mM NaCl, 50 mM Sodium Phosphate, 5 mM EDTA, pH 7.4) and a temperature of 55 degrees to 60 degrees Celsius are suitable. For example, in one embodiment, “moderately stringent conditions” can be defined as hybridizations carried out as described above, followed by washing in 0.2×SSC and 0.1% SDS at 42 degrees Celsius (Ausubel et al., 1989, Current Protocols for Molecular Biology, ibid.).

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (low stringency), more preferably, between about 28° C. and about 42° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

In one embodiment of the present invention, any amino acid sequence described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

Various aspects of the present invention are described in the following experiments. These experimental results are for illustrative purposes only and are not intended to limit the scope of the present invention.

### EXAMPLES

### Example 1

This example describes unexpected and crucial physiological functions of C15/C17ISO in C. elegans, which are indicative of the important role of mmBCFA in other eukaryotes.

### Materials and Methods

### RNA Interference by Feeding

The RNAi feeding vectors were either made in the inventors' laboratory using Taq PCR and cloning genomic fragments into a double T7 vector, pPD129.36 (gift of A. Fire) or obtained from the C. elegans whole genome RNAi feeding library (J. Ahringer, MRC Geneservice).

The RNAi feeding strain was E. coli HT115 transformed with either empty pPD129.36 vector (controls) or with dsRNA-producing constructs. Unless stated differently, wild type N2 Bristol animals were plated as synchronized adults. To obtain synchronized worms of various stages, a large quantity of N2 gravid adults were collected, bleached, and grown to the required stage on HT115 that had been transformed with pPD129.36 (control).

### Gas Chromatography (GC) Analysis

A mixed population of well-fed worms were washed off the plates with water, rinsed 3-4 times, and, after aspirating away water, were frozen at −80° C. Fatty Acid Methyl Esters and lipid extraction were performed as described (Miquel and Browse 1992). GC was performed on the HP6890N (Agilent) equipped with a DB-23 column (30 m×250 μm×0.25 μm) (Kniazeva, Sieber et al. 2003). Each experiment was repeated at least five times. Average values and standard deviations were then calculated for each of the compounds in the experiments.

### Staging Worms to Test for FA Composition

After bleaching gravid adults, an aliquot of the eggs was set apart, and the rest were incubated overnight in M9 at room temperature. On the next day, an aliquot of L1 was frozen for GC analysis. The rest of L1 was plated on agar plates. Subsequently, L2, L3, L4, young adults, and adults along with hatched L1 were collected as the separate samples. Mixed populations of worms starved for 24 to 100 hours were included in the experiment to monitor a possible effect of the starvation.

### Phenotype Rescue Using FA Supplements

Ninety μl of the 4 mM solution of FA (Sigma) in 1% NP40 or 10% DMSO was dropped on the side of the bacterial lawn that contained either elo-5 dsRNA-producing plasmid or the control HT115 vector. Two synchronized young adults were plated and their progeny was scored 4 and 5 days later. Each experiment was performed in at least 30 replicates. For recovering elo-5(RNAi) worms from L1 arrest, wild type adults were placed on the elo-5(RNAi) plates. Four days later, their progeny was removed and eggs of the next generation were left on the plates. Hatched L1 were kept for two or four days before transferring as agar chunks to new elo-5(RNAi) plates. FA supplements were added to spots next to the chunks. Ten plates were prepared for each FA supplement. Control plates contained no supplements. To verify that an addition of supplements does not affect RNA interference per se, we used let-418(RNAi) animals which have sterile phenotype as a control. Neither C15 nor C17 mmBCFA added to let-418(RNAi) plates modified the expected phenotype.

### Designing of GFP Reporter Constructs

To prepare the GFP fusion constructs, genomic fragments were PCR amplified and cloned in frame into one of the GFP fusion vectors (gift from A. Fire). The location of the genomic fragment s and PCR primers used are listed below:

(1) elo-5Prom::GFP: starting at 3.894 kb genomic upstream of the first codon and ending on four bases into the first exon; primers: F-BamHI-tttaggtcattttttgagtcgcca (SEQ ID NO:1) and R-BamHI-tagtctggaattttgaaattgaacgg (SEQ ID NO:2); vector: pPD95.69.

(2) elo-6Prom::GFP: a 4.764 kb fragment covering 3,104 bp upstream and 1660 bp downstream of the predicted start codon and ending on 14 bp into the third exon; primers: F-Sph1-gcccttggaaaccatctacgacgaatc (SEQ ID NO:3) and R-Sma1-tccgaacagaacgacataagagattcc (SEQ ID NO:4); vector: pPD95.77.

(3) acs-1Prom::GFP: a 3.142 kb genomic fragment containing 3,048 kb up-stream of the first predicted ATG and ending on 24 bp into the second predicted exon.; primers: F-SphI-cataattactattgcgtcacatg (SEQ ID NO:5) and R-SphI-ctcttccaaactggcgatgtcga (SEQ ID NO:6) primers; vector: pPD95.69.

(4) pnk-1Prom::GFP: an 1.14 kb fragment that includes 937 bp upstream of the first predicted codon of the C10G11.5 and 203 bp downstream ending on 24 bp into the second exon; primers: F-SphI-tcgtacgatcggaccataggctaa (SEQ ID NO:7) and R-SphI-ctgatcctctgtagcagcggccct (SEQ ID NO:8); vector: pPD95.69.

These constructs were injected into C. elegans at 10-50 ng/μl to form extra-chromosomal arrays. In the case of acs-1, the extra-chromosomal array had been integrated into the C. elegans genome.

Staining Chemosensory Ciliated Neuron with DiI

Worms were soaked in 5 μg/ml solution of DiI (Molecular Probes) in M9 buffer for 1 hour. They were then rinsed three times with M9 and visualized by fluorescence using the Texas Red filter.

### Correlation Analysis

The FA quantities obtained by GC were expressed as percentage of total. T-test (two-tailed distribution) and correlation analysis were performed using the Microsoft Excel® program.

### Visualization and Scoring of the GFP Expression in Promoter::GFP Lines

Synchronized adults were placed on control (HT115 bacterial strain transformed with empty vector, pPD129.36) and RNAi (HT115 bacterial strain transformed with dsRNA construct) plates. Several worms of the next generation were picked from the control and RNAi plates and mounted on the same microscopic slide. GFP images were obtained with the fixed settings and exposure.

### Microarray Analysis

One young adult of the N2 Bristol strain were plated on each control and RNAi feeding plates. Control plates contained E. coli HT115 strain transformed with empty pPD129.36 vector. Experimental RNAi plates contained E. coli HT115 transformed with corresponding dsRNA constructs. The growth conditions, RNA preparations, and data analyses are described below.

### Array Design

GeneChip® C. elegans Genome Arrays (Cat. #900383 Affimetrix) were used, prepared with in situ synthesized 25-mer oligonucleotides.

Samples used:

Organism: Caenorhabditis elegans

Strains: N2 Bristol and elo-5(RNAi), spt-1(RNAi).

Sex: Hermaphrodites

Age: Mixed.

Organism parts: Whole animals.

Quality control: Two replicate samples were obtained for each type of conditions (control and RNAi feeding). The samples were processed entirely independently in parallel experiments starting from plating the worms. One hybridization per sample was used. 3′/5′ ratio for GAPDH and beta-actin were less then 3 in all hybridizations.

Spike controls: BioB was called present and BioC, BioD, and CreX controls were present in increasing intensities in all hybridizations.

### Experimental Design:

Goal: An identification of genes that change their expression level in response to a disruption of elongation of mono-methyl Branched-Chain Fatty Acid (mmBCFA) in C. elegans.

Experimental conditions: Wild type N2 Bristol strain was compared with the elo-5(RNAi) strain. elo-5 encodes the mmBCFA elongation enzyme essential for the mmBCFA biosynthesis.

Growth conditions and preparation of control/reference samples: The RNAi feeding strain was E. coli HT115 transformed with either the empty pPD129.36 vector (control, gift of A. Fire) or with the dsRNA-producing constructs. Worms were cultured at 20° C. One wild type young adult (P₀) was placed on each plate. The population growth was monitored using a dissecting scope. Animals were harvested at three time points between 3^{rd} and 4^{th} day after plating P₀. To generate Sample I, worms were washed off the plates when the F1 population consisted of mostly adults and the F2 generation consisted of mostly L1, some L2 larvae and eggs. For Sample II, worms were washed off the plates several hours later when the F2 generation was enriched with L2. For Sample III, worms were maintained on plates until the F2 generation was represented by a mixture of L2, L3, and L4 larvae, as well as some young adults.

Growth conditions and Preparation of RNAi-treated samples: One wild type young adult was placed on each experimental RNAi plate. The elo-5(RNAi) and spt-1(RNAi) worms were harvested on 4^{th} day after plating P₀ when the F1 generation consisted of mostly adults and the F2 generation consisted of mostly L1 larvae (or mostly L1 and L2 in the case of spt-1(RNAi)).

### Total RNA Isolation

Worms were collected in 15 ml conical tubes and rinsed 5 times in dH₂O followed by a treatment with the TRIAZOL reagent according to the manufacturer's protocol.

### Hybridization and Data Processing

RNA probes preparation were done according to the Affymetrix GeneChip® Protocol in the University of Michigan Microarray Facility.

The C. elegans GeneChip® (Affymetrix) hybridizations were done according to the Affymetrix protocols on the company's equipment in the University of Michigan Microarray Facility.

### Measurement Data and Specifications

Scanning hardware and software: Affymetrix GeneChip® Operating Software (GCOS) Version 1.0 was used for the control of GeneChip Fluidics Stations and Scanners, for data acquisition, sample management, for experimental information, and for gene expression data analysis.

### Statistical Algorithms

Microarray Suit v.5 (Affymetrix) software was used for single array analyses. It utilizes the One-sided Wilcoxon's Signed Rank test as a statistical method to generate the Detection p-values and One-Step Turkey's Biweight Estimate to calculate signals. The inventors performed the global scaling (all probe sets) to the target intensity (TGT) of 100, suggested by Affimetrix® protocol, and filtered out signals that were less than 70 because it was the average signal obtained with the BioB probe defining the minimal sensitivity of the assay (Affimetrix® protocol) in some hybridizations. The signals with Detection p-value>0.05 were also filtered out.

The Data Mining Tool (DMT) software (Affymetrix) was used for the comparison analysis (experiment vs. baseline arrays). Unpaired T-test without corrections was utilized to estimate significance of the difference between two means, where mean is an average signal between replicates for each of controls and experiments. Change p-value>0.05 was chosen as a cut-off. Fold Change was calculated and the transcripts that have their expression level changed>1.57 fold were considered. This arbitrary cut off was relatively low yet potentially detectable in future conformation tests.

Filtered data sets representing comparisons between two conditions were saved as Excel files. For further data manipulations we used MatLab program and custom-made scripts (A. Kniazev, personal communication).

### Microarray Data Manipulation and Analysis

To simplify the task of finding genes differentially expressed in response to the elo-5 RNAi-treatment, but not in response to the stage-regulated differences, the inventors identified the latter in their control samples. The inventors compared control samples and found 1609 genes differentially expressed between the most distant Sample I and Sample III and 287 genes differentially expressed between Sample I and Sample II. The genes differentially expressed between stages were removed from the list of candidate genes. The most populated group of the genes with changed expression is a family of collagens known to be heterochronic genes in C. elegans.

A number of differentially expressed collagens were used for a measurement of similarity between the samples; the smaller the number, the fewer developmental differences observed between the compared samples. Using this “collagen-number” method, the inventors chose Sample I as a reference (or baseline) control for the elo-5(RNAi) and spt-1(RNAi) experiments.

The genes that had changed expression in both elo-5(RNAi) and spt-1(RNAi) samples when compared to the control (Sample I) were identified, after which these were subtracted from the list of the candidates (Table S1). This step excluded a number of genes that may have non-specific changes in their expression, due to a “general” sickness, for example. Two hundred nine genes ended up on the list of candidates that presumably changed their expression level in response to the RNAi-mediated suppression of elo-5 (Table S1).

The genes were re-annotated using the updates from WormBase and NCBI BLAST and Entrez. After the analysis, 41 genes still remained unclassified.

For further analysis, 25 genes were selected that encode proteins related to transcription regulation, lipid metabolic enzymes, intestinal and membrane proteins, and genes that were reported to have RNAi phenotypes similar to that of elo-5(gk208).

In addition, the original data (genes differentially expressed in elo-5(RNAi) as compared to control Sample 1 minus developmentally regulated genes) was used as a reference in order to look for other potential genes of interest. In particular, the expression of lpd-1 was checked and found to be increased in elo-5(RNAi). This gene was included in the short list of candidate genes.

Each of the candidates from the short list was functionally tested for its relationship with mmBCFA metabolism by RNAi followed by GC analysis of the FA composition.

### Results/Discussion

C. elegans Synthesizes Branched-Chain FA De Novo and Uses Two FA Elongation Enzymes to Produce C15ISO/C17ISO

In characterizing FA elongation in C. elegans, the inventors identified eight sequences homologous to the yeast long-chain FA elongation enzymes (Kniazeva, Sieber et al. 2003). To test for their possible functions in vivo, the inventors applied RNAi to the corresponding genes followed by an analysis of FA composition in whole animals using Gas Chromatography (GC). RNAi treatment of four genes—elo-3 (D2024.3), elo-4 (C40H1.4), elo-7 (F56H11.3), and elo-8 (Y47D3A.30)—did not produce any notable phenotypes, whereas suppression of elo-1 (F56H11.4) and elo-2 (F11E6.5), affected the elongation of straight long-chain saturated and polyunsaturated FA (Kniazeva, Sieber et al. 2003).

Surprisingly, the RNAi treatment of the two remaining genes, elo-5 (F41H10.7) and elo-6 (F41H10.8), affected the levels of branched-chain FA. Transcriptional reporter constructs (elo-5Prom::GFP and elo-6Prom::GFP) indicated that both genes are expressed in the gut (data not shown). In addition, elo-5 was expressed in unidentified head cells and elo-6 was expressed in neurons, pharynx, and vulva muscles.

The RNAi of elo-6 significantly reduced the amount of only C17ISO, while the RNAi of elo-5 dramatically reduced quantities of both C15ISO and C17ISO (FIGS. 2A and 2B; arrowheads point to the peaks corresponding to C15ISO and C17ISO). These results indicated that ELO-5 might be involved in the biosynthesis of C15ISO and possibly also C17ISO, whereas ELO-6 may function in elongating C15ISO to C17ISO (FIGS. 2C and 2D). FIG. 2C shows a comparison of fatty acid (FA) composition in three strains; wild type, elo-5(RNAi) and elo-6(RNAi). C17ISO is decreased in both RNAi strains, while C15ISO is only decreased in elo-5(RNAi). FIG. 2D shows the suggested elongation reactions catalyzed by ELO-5 and ELO-6 in the C15ISO and C17ISO biosynthesis. Fatty acids are elongated by an addition of two carbon groups at a time. These data suggest that ELO-6 acts at the elongation step from C15 to C17, whereas ELO-5 appears to be involved in the production of both C15ISO and C17ISO. To the best of the inventors' knowledge, these are the first enzymes that have been shown to be involved in the long-chain mmBCFA biosynthesis in a non-bacterial in vivo system and the first enzymes of the long chain FA elongation family related to mmBCFA production.

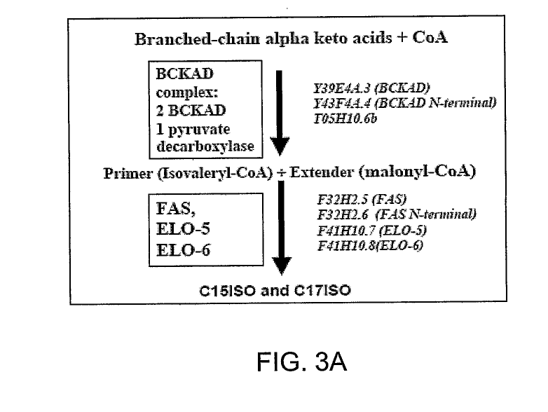

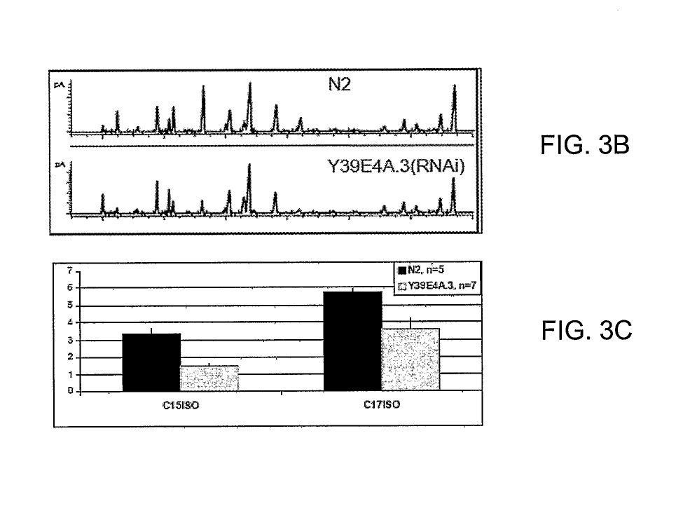

In bacteria, the mmBCFA biosynthesis utilizes branched-chain α-keto-acids of leucine, isoleucine, and valine to produce mmBCFA acyl-CoA primers that substitute for acetyl-CoAs in the conventional FA biosynthesis (Oku and Kaneda 1988). Key enzymes engaged in synthesizing the mmBCFA acyl-CoA primers are branched-chain aminotransferase (BCAT) and the branched-chain α-keto-acid dehydrogenase (BCKAD) complex (FIG. 3A). The elongation of the mmBCFA backbone is then carried out by fatty acid synthetase (FAS). FIG. 3A shows the early steps of the mmBCFA biosynthesis in bacteria, based on (Oku and Kaneda 1988) (BCAT, branched-chain aminotransferase; BCKAD, branched-chain alpha-keto acid dehydrogenase; IVD, isovaleryl-CoA dehydrogenase; FAS, fatty acid synthetase). The predicted corresponding C. elegans genes encoding predicted orthologs were identified (shown in italicized names of reading frames).

An ability of C. elegans to grow on the chemically defined axenic media CbMM, which lacks the potential mmBCFA precursors, has suggested that the animals can synthesize mmBCFA de novo. If so, a disruption of the BCKAD complex could affect mmBCFA levels. The inventors identified a predicted C. elegans protein, Y39E4A.3, with a significant sequence homology to E1 alpha subunit of BCKAD (Y39E4A.3 scores 8e-50 on 57% of the length with the Bacillus subtilis BCKAD, and 1.4-e134 on 88.4% of the length with the Homo sapience BCKADs). RNAi of Y39E4A.3 led to a significant decrease in C15ISO and C17ISO production (FIG. 3B; black arrowheads point to C15ISO and C17ISO and FIG. 3C; p-value is 0.001 and 0.008 for C15ISO and C17ISO, respectively). RNAi suppression of another predicted component of the BCKAD complex, pyruvate dehydrogenase (T05H0.6), resulted in a similar decrease in C15ISO and C17ISO (data not shown), indicating a role for the C. elegans BCKAD protein in long-chain mmBCFA biosynthesis. Thus, C. elegans appears to use the same initial reactions to produce mmBCFA as bacterial cells. In addition, the worms use enzymes of the FA elongation family, ELO-5 and ELO-6 to complete the pathway.

A connection between BCKAD functions and mmBCFA quantities has been previously reported in humans (Jones, Peet et al. 1996). Normally hair fibers are densely covered with C21anteISO, which contributes about 38.2% to the total hair FAs (Jones and Rivett 1997). It was observed that patients with the Maple Syrup Urine Disease (MSUD), which is caused by an inherited mutation in the BCKAD gene, had a drastically reduced level of mmBCFA in their hair. Together, these data suggest that the long-chain mmBCFA biosynthesis could be similar in bacteria, C. elegans, and human.

### Blocking ELO-5 Function Causes Growth and Developmental Defects

While the suppression of the elo-6 activity by feeding dsRNA to wild type animals did not cause obvious morphological or growth defects, the suppression of elo-5 resulted in a more pronounced phenotypes (data not shown). Worms originating from wild type eggs laid on the elo-5(RNAi) plates displayed no obvious growth or morphological abnormality until the second day of adulthood when they developed an egg-laying defect (data not shown). Eggs of the next generation hatched on time but the progeny arrested at the first of the four larval stages (L1). The small larvae maintained morphological integrity and could survive on a plate for up to 3-4 days. The arrest was only observed in progeny of parents exposed to elo-5 RNAi at the L1 stage.

When parental animals were subjected to elo-5 RNAi at later larval stages (L2-L4), their progeny did not arrest in L1 but continued to develop into adulthood. These animals had no obvious defects in locomotion, pharyngeal pumping, intestinal contractions, chemotaxis response, touch sensitivity or general anatomy (data not shown). However, the growing worms became progressively sick (data not shown). The gonads appeared normal at the L4 and early adult stages, but after fertilization of 1-10 oocytes, oogenesis became impaired. Gonad degeneration began with a pronounced vacuolization in the mid-section of the gonad followed by the appearance of disorganized clumps of nuclei in the proximal part. An egg-laying defect became apparent and only a few progeny arose from these worms, which then arrested at L1. The development of the elo-5 RNAi phenotypes is likely due to a gradual elimination of the ELO-5-associated functions. These data suggest that these functions are crucial for larval growth and development.

The inventors also obtained a likely null mutant of the elo-5 gene, elo-5(gk208) that has a 245 bp deletion eliminating the predicted first exon (Genome Science Center, BC Cancer Research Center, Vancouver). This allele phenocopies the L1 arrest phenotype of the elo-5(RNAi) animals.

A Deficiency of C15/C17ISO FA is Solely Responsible for the Defects Caused by elo-5(RNAi)

The inventors reasoned that if the defects observed in the elo-5(RNAi) animals resulted directly from the deficiency of C15ISO and C17ISO, then feeding these worms with C15ISO and C17ISO should mask a shortage of endogenous C15/C17ISO and permit the animals to grow normally. As predicted, the C17ISO as well as C17anteISO supplements rescued the elo-5 RNAi defects (52/60 and 58/60 plates correspondingly). A partial rescue was observed on the plates supplemented with C15ISO and C15anteISO (23/38 and 20/28 plates correspondingly). Corroborating results were obtained when homozygous elo-5(gk208) animals were supplied with C17ISO grew normally. In sharp contrast, neither saturated, mono- or poly-unsaturated FA molecules (C16:0, C16:1 n7, C17:0, C18:3 n6) nor mmBCFA with shorter or longer backbones (C13ISO, C18ISO, C19ISO), nor poly-methyl branched phytanic acid were able to rescue or reduce defects (0/30 plates in each experiment). Therefore, the inventors have determined that only dietary 17-carbon mmBCFA are competent to bypass the biochemical defect caused by loss of ELO-5 function.

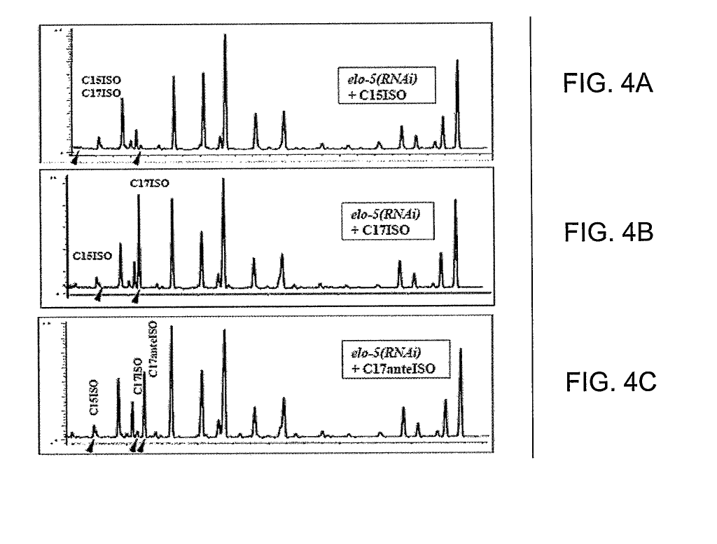

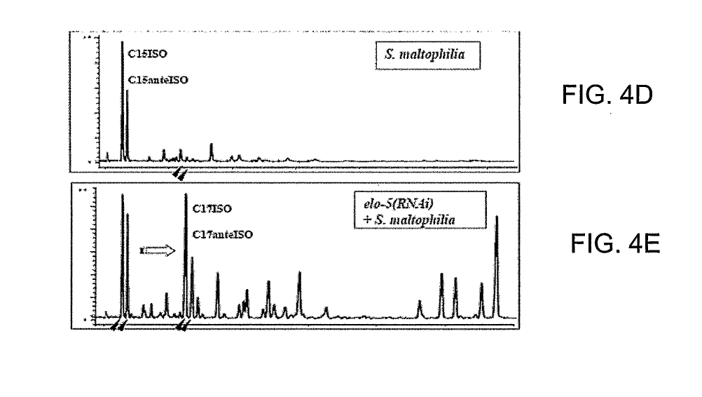

GC analysis of FA composition in worms grown on supplemented plates revealed that only C17ISO and C17anteISO are significantly incorporated into lipids (FIGS. 4A-4C). FIG. 4A shows that animals grown with C15ISO supplements were partially rescued to wild type phenotype; however, no accumulation of C15ISO or its elongation to C17ISO was detectable. FIGS. 4B and 4C show that animals grown with the C17ISO (FIG. 4B) and or C17anteISO (FIG. 4C) supplements were fully rescued (peaks corresponding to C71ISO and C17anteISO are prominent). Because the addition of C15ISO did not result in elongation to C17ISO (FIG. 4A), the inventors wanted to determine whether ELO-6 was capable of extending an FA backbone in the absence of ELO-5, or whether the supplied free mmBCFA molecules could enter a different metabolic pathway, for instance, a degradation pathway. To distinguish between these two possibilities, the inventors added mmBCFA-producing bacteria on top of the regular RNAi feeding E. coli strain (HT115) that lacks mmBCFA. This mmBCFA-producing strain was identified by chance; the inventors noticed that in the presence of a certain bacterial contaminant the animals could overcome the elo-5(RNAi) effects. Using a rapid bacterial identification method, the inventors determined the contaminant to be Stenotrophomonas maltophilia. GC analysis revealed that this bacterial strain produced a high quantity of C15ISO and C15anteISO but not 17-carbon mmBCFA (FIG. 4D; arrowheads point to major FA, C15ISO and C15anteISO). GC analysis of elo-5(RNAi) animals fed with S. maltophilia indicated that they not only accumulated bacterial C15ISO and C15anteISO but also efficiently elongated these FA species to C17ISO and C17anteISO that are absent in S. maltophilia (FIGS. 4D and 4E; arrowheads indicate mmBCFA, and arrow illustrates the elongation from C15 to C17 mmBCFA). This suggested that elongation from C15ISO to C17ISO mmBCFA is not impaired in the elo-5(RNAi) animals. Therefore, ELO-6 function remains intact in elo-5(RNAi). Apparently, C15ISO added to the plates could not be utilized by ELO-6 whereas C15ISO-CoA and/or C15anteISO-CoA originating from the bacterial food could, suggesting that free and esterified mmBCFA were likely to enter alternative pathways.

The essential roles of C15/C17ISO were also supported through an examination of the elo-5(gk208) deletion mutant. The homozygous mutants grew without any obvious morphological defects when maintained on the plates supplemented with C17ISO or seeded with S. maltophilia. However, removal of the mmBCFA supplements or S. maltophilia by bleaching resulted in the same L1 arrest phenotype as the elo-5(RNAi) worms.

L1 Arrest of the elo-5(RNAi) Animals is Reversible and Related to the Variations in Levels of C17ISO During Development

The inventors then asked if elo-5(RNAi) animals arrested at L1 stage could be recovered by adding the 17-carbon mmBCFA supplements. Indeed, C17ISO and C17anteISO could effectively release L1 larvae from the developmental arrest; about 50% of two day-arrested and 1% of four-day-old L1 were rescued to full growth and proliferation. Since C17anteISO could not be detected in the laboratory animals under normal conditions of culturing, C17ISO appeared to be the principal molecule conveying the ELO-5 function. Therefore, the L1-arrest of the C17ISO-depleted worms is both completely penetrant and reversible, indicating that C17ISO plays a critical role in growth and development at the L1 stage.

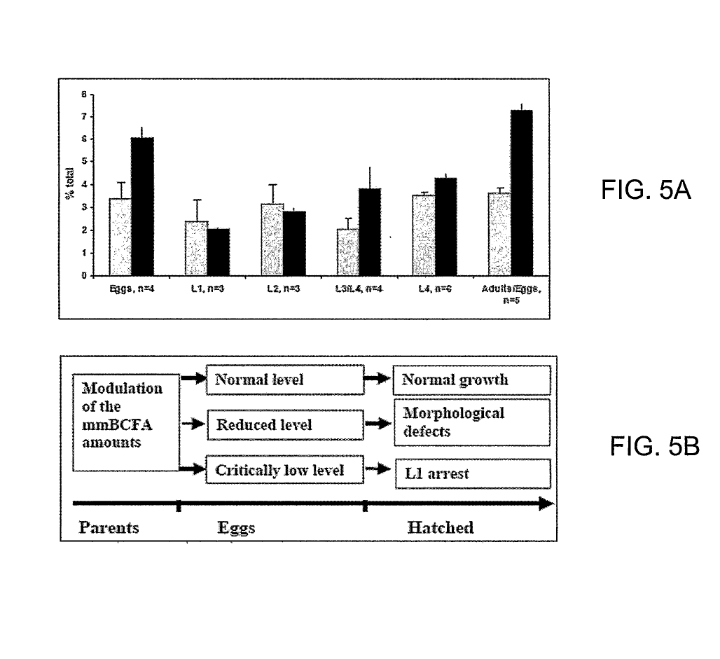

The analysis of the FA levels of staged worms revealed that the C17ISO level increases gradually from a relatively low level at L1 to its peak in gravid adults containing eggs (FIG. 5A). Specifically, FIG. 5A shows the relative amounts of C15ISO and C17ISO in the worm samples collected in different developmental stages. The amount of the mmBCFA molecule is presented as the percentage of total FA in each sample. Based on the analysis of GFP reporter constructs (data not shown) and in situ hybridization data (results from NextDB by Y. Kohara, Tokyo), neither elo-5 nor elo-6 are significantly expressed in eggs or L1. Therefore, C17ISO likely accumulates in embryos during oogenesis. It may be directly transported from gut to gonads since both ELO-5 and ELO-6 were expressed mainly in the gut and because feeding C17ISO rescued the elo-5 mutant phenotypes. When RNAi-mediated disruption of elo-5 occurs at the L1 stage of a parent and consequently blocks C17ISO synthesis from that stage on, the eggs and L1 animals of the next generation are expected to contain a critically low concentration of C17ISO, halting further development. Because the arrested L1 can be rescued by a dietary supply of the mmBCFA, the deficiency is not likely to cause critical defects during embryonic and early postembryonic periods.

If elo-5 RNAi is applied to the parent worms at or after the L2 larval stage, when the amount of C17ISO has already been elevated and/or the RNAi-effect is less penetrant, the progeny may receive sufficient C17ISO to pass the L1 arrest stage. The resulting animals, however, become visibly unhealthy at L4 and adult stages as mentioned earlier, suggesting that C17ISO also plays a role in late developmental stages.

Based on these results, the inventors propose a relationship between the amounts of C17ISO and developmental stages (FIG. 5B). As shown in FIG. 5B, depending on the time of RNAi onset, the amount of C17ISO in F1 eggs varies. If elo-5 is suppressed in parental animals after they have begun to synthesize mmBCFA, then their eggs will have a reduced C17ISO level that is still above the critical low level which permits these animals to grow but display gonadal defects. These worms produce a small number of progeny that is then arrested in L1. If parental animals are treated with elo-5(RNAi) right after hatching, they are unable to initiate the mmBCFA biosynthesis and the levels of C15ISO and C17ISO in their eggs are reduced to below the critical low level, resulting in L1 arrest of their progeny. In this model, the level of C71ISO is monitored at the first larval stage and the decision is made whether to proceed or pause in development. The analysis of GC data from staged animals has also indicated that the variation of C17ISO level is correlated with only two other FA species, suggesting a potential compensatory and co-regulation mechanism.

The C17ISO Level Correlates with the Levels of Two Other FAs During Development

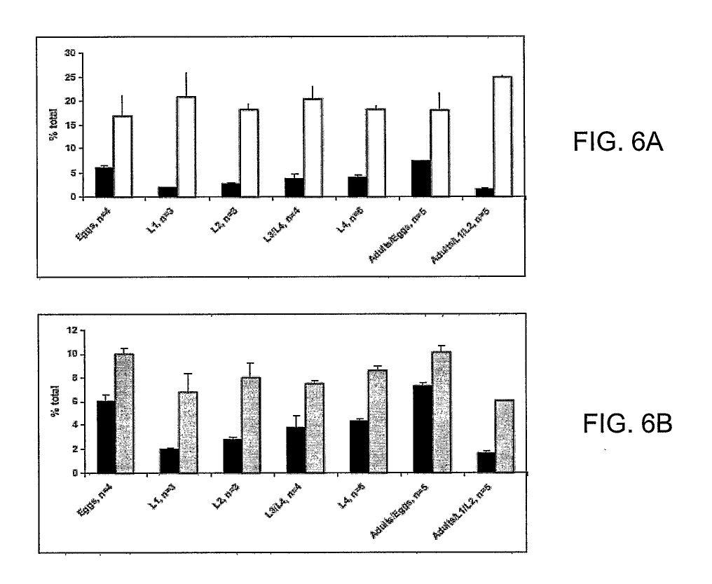

FA homeostasis implies that relative amounts of various FA species are coordinated and balanced for optimal performance. To obtain information that may reveal why and how numerous FAs and their specific metabolic enzymes are maintained in nature, the inventors carried out analysis to determine a possible correlation between changes in the levels of C17ISO and other FA detected in worms. The inventors have analyzed a large amount of GC data (n=50) obtained from mixed populations of wild type animals where the fractions of eggs, larvae, and adults randomly varied. The GC data was separately obtained from staged worms was included: eggs, L1, L2, L3, L4, and gravid adults. The inventors found that the amounts of C17ISO significantly correlated with only two other FA molecules: linoleic, C18:2 n6, and vaccenic, C18:1n7 (FIG. 6). The graphical illustrations in FIG. 6 were obtained by GC analysis of synchronized populations of worms. Changes in relative amounts of FA are emphasized with treadlines created in Microsoft Excel. Combined with the GC measurements generated from additional 50 samples (material and methods), these data were used to calculate correlation coefficients (CORREL_{C17ISO/C18:2n6}=+0.82772, T-TEST=6.54814E-07 and CORREL_{C17ISO/C18:1 n7}=−0.85162, T-TEST=4.74094E-05). A potential physiological significance of these correlations is intriguing.

The observed negative correlation between the levels of C17ISO and C18:1 n7 throughout development may indicate a compensatory adjustment important for physiological functions, such as retention of the cell membrane physical properties. mmBCFA and monounsaturated straight-chain FA have been previously implicated in regulating membrane fluidity, which depends on the ratio of saturated FA to monounsaturated and branched-chain FA content in bacterial cells. An elevation in monounsaturated FA amounts in response to the decrease of BCFA but not vise versa was observed in Streptomyces avermitilis, suggesting that monounsaturated FA may sense a state of membrane fluidity.

In the elo-5(RNAi) treated worms, a substantial loss of C15/C17ISO is also accompanied by a change in the FA composition, most noticeably by the elevation in C18:1n7 (FIG. 2C), a result consistent with the above observation. To estimate the effect of the C15/C17ISO deficiency on the membrane saturation, the saturation index (SI=[saturated FA]/[mmBCFA+monounsaturated FA]) was calculated. No significant differences were detected in elo-5(RNAi) worm compared to wild type (SI=0.325±0.011, n=6 and SI=0.320±0.032, n=5 respectively). Therefore, elo-5(RNAi) may not cause a massive cell membrane dysfunction.

A positive correlation between the amounts of C17ISO and that of C18:2 n6 may suggest a potential common function during development. In addition to the importance of linoleic acid as a substrate for PUFA biosynthesis, its hydroxylated fatty acid derivative (HODEs) is known as a signaling molecule affecting chemotaxis, cell proliferation, and modulation of several enzymatic pathways. A correlation between C17ISO and linoleic acid may also suggest a similar regulation of biosynthesis of the two molecules.

The changes in the FA composition associated with a decrease in C15/C17ISO indicate that the metabolism of straight-chain FA species is responsive to the mmBCFA levels and suggest a cross regulation. Interestingly, in the elo-5(RNAi) animals fed with the C15ISO/C15anteISO containing bacterial supplement (S. maltophilia), the FA composition is significantly altered (FIG. 4E). It appears that mmBCFA become principal components in a range of 16-18 carbon FAs. This suggests that large quantities of mmBCFA are not toxic. In contrast, because these worms grow and proliferate well, mmBCFA seem to be efficient substitutes for saturated and monounsaturated straight-chain FAs.

### The Worm SREBP Homology Controls Production of Branched-Chain FA

In mammals, straight-chain FA biosynthesis depends on the 1c-isoform of sterol regulatory element binding protein, SREBP-1c, which promotes the expression of FA metabolic enzymes. There is only one protein in C. elegans that is homologous to mammalian SREBPs, Y47D3B.7 (the gene has been named lpd-1 for LiPid Depleted 1) (McKay, McKay et al. 2003). McKay and co-authors have shown that worms treated with lpd-1 RNAi display lipid-depleted phenotype. They have also shown that lpd-1 regulates the expression of several lipogenic enzymes, Acetyl-CoA Carboxilase (ACC), Fatty Acid synthetase (FAS) and Glycerol 3-Phosphate Acyltransferase (G3PA) (McKay, McKay et al. 2003). Thus, similar to its mammalian homolog, lpd-1 is involved in straight-chain FA biosynthesis.

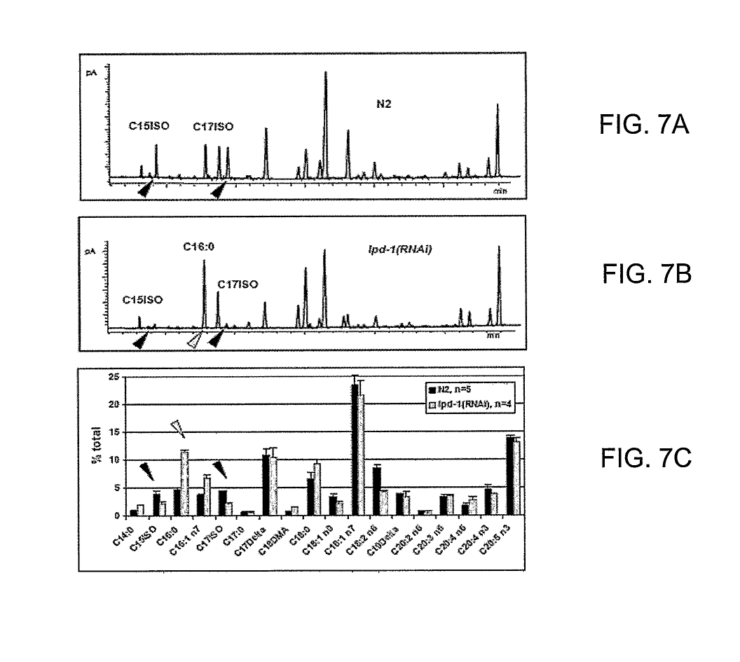

The inventors wanted to see if lpd-1 also plays a role in mmBCFA metabolism. RNAi was first applied to lpd-1 and the FA composition of the mutant worms was determined. As expected, the FA content of treated animals was significantly changed, but surprisingly the most reduced were the levels of C15ISO and C17ISO (FIGS. 7A-7C). Also significantly reduced was the amount of C18:2 n6. In contrast, the C16:0 level was elevated. FIGS. 7A and 7B show the GC profiles of wild type and lpd-1(RNAi)-treated worms, respectively. FIG. 7C shows a summary of several independent GC runs (bars represent the percentages of total FAs). The results show that the levels of C15ISO, C17ISO, and C16:0 are significantly altered by the RNAi treatment (black arrowheads point to differences in the C15ISO and C17ISO amounts. Grey arrowhead indicates the changes in palmitic acid, C16:0). These data indicated that, in addition to regulating the first steps of global FA biosynthesis through the activation of the ACC and FAS transcription, the worm SREBP homolog regulates mmBCFA elongation as well as desaturation of straight-chain FA.

As reported previously, disruption of lpd-1 through a mutation or RNAi injection caused early larval arrest (McKay, McKay et al. 2003). The effect of lpd-1 RNAi feeding in the inventors' experiments was apparently less severe. The RNAi-treated animals displayed slow growth, morphological abnormalities, and egg-laying defects but no larval arrest. Supplementing C17ISO to the plates did not significantly rescue these defects.

### LPD-1 and LPD-2 Diverge in Functions

LPD-2 (C48E7.3) is another C. elegans homolog of a mammalian lipogenic transcription factor, CCAAT/enhancer-binding protein (C/EBP). McKay and co-authors have shown that the lpd-2(RNAi) and lpd-1(RNAi) phenotypes are quite similar; affected worms are defective in growth, pale and scrawny in appearance and in lack of fat content (McKay, McKay et al. 2003). They have also shown that LPD-1 and LPD-2 control the expression of the same lipogenic enzymes: ACC, FAS, ASL, and G3PA. The inventors tested to see if LPD-1 and LPD-2 function similarly in the regulation of mmBCFA biosynthesis. In contrast to the result from lpd-1(RNAi), the FA composition in lpd-2(RNAi) worms was not significantly different from that of wild type animals even though these animals had a noticeably sick appearance (data not shown). This result suggested that, in addition to having some common targets, LPD-1 and LPD-2 have distinct functions. LPD-1 is important for production of mmBCFA as well as other very long-chain FA, whereas LPD-2 has no specificity for any particular type of FA.

elo-5 ad elo-6 are Likely Targets of LPD-1

The changes in FA composition observed in lpd-1(RNAi) would be consistent with down-regulation of elo-5, elo-6 (decrease in mmBCFA), elo-2 (increase in C16:0) (Kniazeva, Sieber et al. 2003) and Δ9- and/or Δ12-desaturase genes (decrease in C18:2 n6). The genes encoding mammalian orthologs of the C. elegans elo-2 and Δ9-desaturase genes are known targets of SREBP-1c. To examine if elo-5 and elo-6 are targets of lpd-1, the inventors analyzed the expression of elo-5, elo-6, and lpd-1.

Evaluation of the expression from a lpd-1Prom::GFP fusion construct (a gift of J Graff) in transgenic animals revealed that, in addition to the previously reported expression in intestinal cells (McKay, McKay et al. 2003), the construct is strongly expressed in a subset of head neurons (data not shown). Using a lipophilic dye, DiI, which highlights chemosensory ciliated neurons, we identified these neurons as amphids. In the strains carrying elo-5Prom::GFP and elo-6Prom::GFP reporter constructs, GFP fluorescence was also detected in the gut and several head neurons including amphid neurons (data not shown).

If LPD-1 promotes elo-5 and elo-6 expression, then RNAi of lpd-1 should alter GFP intensity in elo-5Prom::GFP and elo-6Prom::GFP reporter strains. The level of GFP expression driven by elo-5 and elo-6 promoters is high in conventionally cultured animals. In the worms maintained on the lpd-1(RNAi) plates, the expression was noticeably weakened, suggesting a down-regulation of the promoter activities (data not shown). No significant changes in the GFP expression were detected in a control strain containing a kqt-1Prom::GFP construct that also expresses GFP in head neurons and the gut (unpublished).

To test if the disruption of FAS, a target of LPD-1 (McKay, McKay et al. 2003), could contribute to the observed decrease of C15/C17ISO in lpd-1(RNAi), the inventors analyzed FA composition in FAS(RNAi) strains. There is one predicted FAS gene, F32H2.5, and its shorter homolog, F32H2.6 in the C. elegans genome. The latter can only encode the N-terminal portion of the protein. These genes share extended nucleotide identity and RNAi of one could thus possibly affect the other. Consistent with a critical role for FAS in the first steps of FA biosynthesis, the RNAi-mediated disruption of F32H2.5 and F32H2.6 resulted in multiple defects and a lethal growth arrest (data not shown). The FA composition (the content and relative amounts of various FA species) of the affected animals remained, however, unchanged. This suggested that disruption of FAS does not selectively alter FA biosynthesis and that neither FAS protein is specific for mmBCFA. Therefore, down-regulation of FAS by loss of lpd-1 cannot account for the severe deficiency of mmBCFA in lpd-1(RNAi).

Thus, the inventors showed that disruption of lpd-1 affects C15ISO/C17ISO biosynthesis. The fact that lpd-1, elo-5 and elo-6 are expressed in the same cells concurrently and that the GFP reporter analysis indicated that elo-5 and elo-6 transcription is down regulated in the absence of lpd-1 suggests that elo-5 and elo-6 are likely to be the targets of lpd-1.

Since ACC and FAS catalyze the first steps in the biosynthesis of straight-chain FAs while ELO-5 and ELO-6 extend mmBCFA molecules, LPD-1 appears to integrate conventional and “unusual” FA biosyntheses. It seems reasonable to predict that in order to differentiate between these metabolic pathways and mediate compensatory or adaptive changes in FA composition, LPD-1 must interact with other factors such as nuclear receptors activated by specific FA ligands. It is thus important to screen for such interactions to better understand the FA homeostasis in C. elegans.

A Reciprocal Correlation Between the lpd-1 Expression and mmBCFA Levels

Because mammalian SREBP-1c regulates PUFA biosynthesis and is feedback-inhibited by PUFAs, the inventors asked if lpd-1 could be regulated by mmBCFA at the transcriptional level. The microarray data (discussed below) indicated a 1.68 fold up-regulation of lpd-1 in the elo-5(RNAi) animals, while no changes were detected in its levels between samples from wild type animals at different developmental stages (see Materials and Methods above).

To examine the influence of the mmBCFA deficiency on lpd-1 expression, the inventors grew the lpd-1Prom::GFP containing strain on the elo-5(RNAi) and control plates to compare GFP fluorescence. No obvious difference in the GFP expression driven by the lpd-1 promoter in intestinal cells was detected on the elo-5(RNAi) plates versus the control plates. A modest change in the transcription level (1.68 fold) could be masked by a variability of the expression between individual animals and even between individual cells (not shown). In contrast to the observation in the intestinal cells, a strong induction of GFP was detected in amphid neurons of lpd-1Prom::GFP; elo-5(RNAi) animals (data not shown). This suggested that a chronic deficiency of mmBCFA in elo-5(RNAi) animals may transcriptionally stimulate LPD-1 production at least in neuronal cells.

Collectively, the inventors' results suggest that the relationship between lpd-1 and C15/C17ISO is reciprocal; while down-regulation of lpd-1 transcription results in the C17ISO deficiency, the C15/C17ISO deficiency up-regulates lpd-1 transcription at least in a subset of cells. Therefore, the worm SREBP homolog, LPD-1, may play an important role in mmBCFA homeostasis.

Screening for Additional Genes Involved in mmBCFA Homeostasis

Because C15ISO and C17ISO play critical roles in animal development and growth, the inventors suspected mechanisms might exist to respond to and regulate their levels. Regulation of mmBCFA homeostasis may involve transcription factors, metabolic enzymes, as well as transport and binding proteins. It is reasonable to suggest that a deficiency of mmBCFA triggers a compensatory alteration in the expression of these genes. It is also feasible that a comparative analysis of global gene expressions between wild type and mmBCFA deficient animals may reveal these potential changes and the changes underlying developmental and growth functions of mmBCFA.

The inventors used DNA microarray analysis to compare the total gene expression in elo-5(RNAi) and wild type animals. To select candidate genes, restrictive criteria were applied and genes were excluded of which the expression was also changed in the spt-1(RNAi) strain (Materials & Methods). The spt-1(C23H3.4) gene encodes a predicted C. elegans homolog of serine-palmitoyl transferase subunit 1. RNAi of spt-1 strongly affects the FA composition without reducing the C15/C17ISO levels (data not shown). The F1 generation of spt-1(RNAi) animals developed gonadal and egg-laying defects that are similar to the phenotype of F1 animals from parents treated with elo-5(RNAi) at a late larval stage (described earlier) (data not shown). The inventors thought that by deselecting genes that have altered expressions in spt-1(RNAi), they would be able to eliminate variations in gene expressions unrelated to the mmBCFA deficiency. Such variations might emerge from altered straight-chain FA metabolism and from general sickness. Here, the analysis of the first set of candidate genes that are differentially expressed in elo-5(RNAi) and may relate to the C15/C17ISO homeostasis are discussed.

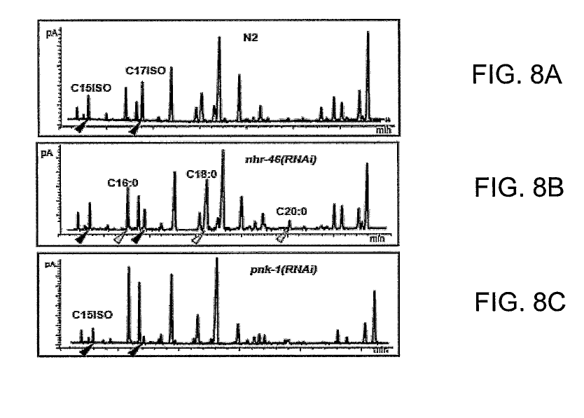

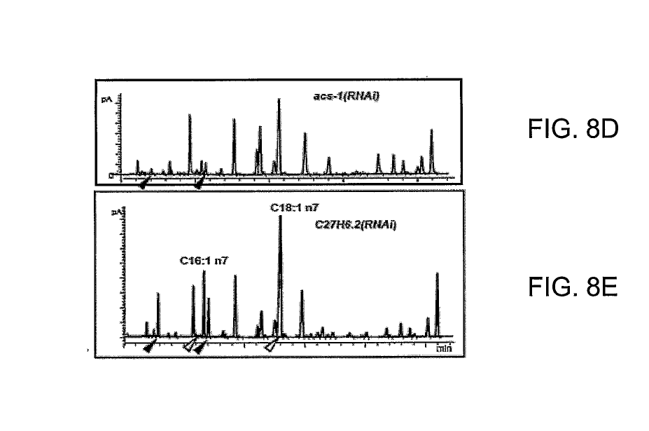

Twenty-five genes were selected in the screen (Table 1) and each was functionally tested by RNAi and GC analysis for its role in C15/C17ISO metabolism. RNAi of four of these genes (pnk-1 (C10G11.5), nhr-49 (K10C3.6), acs-1 (F46E10.1), and C27H6.2) significantly affected the FA composition (FIGS. 8A-8E). All four genes encoded products structurally homologous to the known proteins (PNK-1, human pantothenate kinase; NHR-49, nuclear hormone receptor; ACS-1, very long-chain FA CoA ligase; and C27H6.2, RuvB-like DNA binding protein). Specifically, FIG. 8A shows the GC profile of wild type, and FIGS. 8B-8E show the GC profiles of the RNAi-treated worms. In FIGS. 8B-8D, RNAi of the three genes resulted in a decrease of the C17ISO or both C15ISO and C17ISO levels indicated by black arrowheads. In addition, a significant elevation in straight-chain saturated FA indicated by gray arrowheads is observed in K10C3.6(RNAi). FIG. 8E shows that C27H6.2(RNAi) does not cause significant changes in mmBCFA but results in an elevation of straight-chain monounsaturated FA, C18:1 n7, indicated by white arrowheads. Statistical analysis of several GC runs on each of the sample was also carried out (data not shown).

TABLE 1
Candidate genes and their encoded proteins selected from microarray
data for functional tests (RNAi and GC analysis)

| Gene | Direction/fold | | |
|---|---|---|---|
| Name | of Change² | | Protein properties |
| pnk-1 | up | 2.98 | Pantothenate kinase |
| | down | 1.6 | RuvB-like 1 (49-kDa TATA box-binding |
| | | | protein-interacting protein |
| | up | 1.7 | Acetyl-coenzyme A synthetase |
| | down | 1.99 | Similar to Ras family, GTP-binding |
| tim-13 | down | 1.62 | Zn-finger, mitochondrial |
| | down | 1.58 | 3-oxo-5-alpha-steroid 4-dehydrogenase |
| | down | 1.58 | Zn-finger-like |
| | down | 1.58 | Similar to DEAD-box, initiation factor-helicase |
| | up | 1.6 | Similar to E1-E2 ATPase |
| tlf-1 | down | 1.6 | Transcription factor TFIID like |
| acs-1 | up | 2.72 | Long-chain-fatty-acid-CoA ligase |
| mxl-3 | up | 1.89 | Helix-loop-helix DNA-binding domain |
| | down | 1.7 | Zn-finger, C2H2 type |
| | up | 2.54 | Unknown, intestinal |
| | down | 1.7 | Endoplasmic reticulum targeting sequence |
| nhr-49 | up | 1.68 | Zinc finger, nhr-49, steroid nuclear receptor |
| | down | 1.64 | alpha-beta hydrolase fold, Esterase/lipase/ |
| | | | thioesterase |
| | up | 2.02 | Zinc-finger MYND type, similar to |
| | | | programmed cell deat2 (PDCD2) |
| | down | 1.58 | Zn-finger, C2H2 type |
| | up | 1.75 | LDL receptor-related protein |
| | down | 2.25 | Similar to mitochondrial import receptor |
| | | | subunit TOM22 |
| cyp-1 | down | 1.71 | Peptidyl-prolyl cis-trans isomerase |
| | down | 1.66 | Inner membrane protein |
| | up | 1.64 | Similar to fatty acid amide hydrolase |
| | down | 1.69 | Similar to acyl carrier protein, |
| | | | Phosphopantetheine-binding domain |

¹Predicted open reading frames by the C. elegans Genome Project (WormBase.org)
²Data from comparing arrays from the experimental sample (elo-5(RNAi)) with that from a baseline control sample (Sample I)(see Supporting Materials and Methods).

### Analysis of the Candidate Genes

Circumstantial evidence suggests that these four candidate genes may be involved in feedback regulation of mmBCFA biosynthesis. First, the expression of these genes is not variable in nature as judged by a comparison of the microarray data obtained from developmentally different populations of N2 (Materials & Methods) as well as for vulval development pathway mutants (data obtained for an unrelated project, J. Chen, personal communication). Secondly, the direction of the changes for three of the genes is in concordance with the proposed feedback regulation; pnk-1, nhr-49, and acs-1 were up-regulated in C17ISO deficient elo-5(RNAi). Lastly, a functional analysis shows that these three candidate genes are required for the normal level of mmBCFA production (RNAi of the genes affects the mmBCFA production). The forth candidate gene, C27H6.2, affects the level of vaccenic acid (C18:1 n7), which is related to the levels of mmBCFA (FIGS. 8A-8E), suggesting cross talk between fatty acid biosynthesis pathways.

To detect a potential feedback regulation involving acs-1 and pnk-1, the inventors made reporter strains with GFP expression driven by acs-1 and pnk-1 promoters, acs-1Prom::GFP and pnk-1Prom::GFP, respectively. These two genes showed a higher degree of up-regulation than the other candidates according to the microarray data. In addition, RNAi of these two genes resulted in a significant loss in the mmBCFA fraction. The GFP fluorescence from acs-1Prom::GFP and pnk-1Prom::GFP was readily detectable in the gut. Expression of acs-1Prom::GFP was also detected in the canal-associated neurons (CAN) in the head neurons and vulval cells. A comparison of synchronized animals grown on the control and elo-5(RNAi) plates indicated a significantly brighter fluorescence in the RNAi worms (data not shown) suggesting up-regulation of acs-1 and pnk-1 under C15ISO/C17ISO deficiency. These results were in concordance with the microarray data. Moreover, pnk-1, but not acs-1 seemed to be regulated by LPD-1 because pnk-1Prom::GFP expression was significantly reduced on lpd-1(RNAi) (data not shown).

It was interesting to note that the pnk-1 and acs-1 genes were previously selected in two different screens as potential targets of the daf-2/daf-16 (Y55D5A.5 and R13H8.1 correspondingly) pathway. pnk-1 had been identified in a screen for genes affecting C. elegans life-span and metabolism through analysis of promoter regions and it was confirmed as a direct target of DAF-16, a forkhead transcriptional factor. acs-1 had been identified in a microarray screen for DAF-16 targets that influence the life-span. A third gene, nhr-49, had been previously selected in a screen for fat regulatory genes. It was shown that RNAi of this gene leads to an increase in fat accumulation in affected animals. The inventors' analysis of nhr-49(RNAi) animals showed that reduction of the nhr-49 activity results in up-regulation of saturated FA biosynthesis that may contribute to fat accumulation. Although the regulatory path for this process remains unknown, the involvement of daf-2 has not been ruled out.

A potential link of the candidate genes to DAF-2/insulin signaling is very intriguing. The C. elegans insulin-signaling pathway is involved in sensing nutritional state and metabolic conditions as well as controlling growth and diapause. A described herein, a mmBCFA deficiency causes transient L1 arrest. This phenotype strikingly resembles L1 arrest of worms hatched in the absence of food (a method commonly used to obtain synchronized animals). An investigation of possible roles for mmBCFA in food sensation and insulin signaling pathways is underway (see Examples below).

Down-regulation of the forth candidate gene, C27H6.2, may result in a significant increase of monounsaturated FA levels (FIGS. 8A-8E). This is consistent with the enlarged fraction of monounsaturated FAs observed in the elo-5(RNAi) animals (FIG. 3C). Down-regulation of C27H6.2 may have an adaptive effect to compensate for the loss of mmBCFA in cell membranes. If so, C27H6.2 may be a part of mechanism that senses and tunes physical properties of membranes. C27H6.2 is homologous to an evolutionary conserved protein RuvB/TIP49a/Pontin52 essential for growth and proliferation. Its mammalian ortholog acts as a transcriptional cofactor that binds to β-catenin, TATA-box binding protein, and likely to a number of other diverse transcription factors.

### Conclusions

Two mmBCFA are normally detected in C. elegans: C15ISO and C17ISO. A deficiency of these FA is lethal and cannot be compensated by any other FA present, indicating their crucial importance for growth and development. There are two sources of C15ISO/C17ISO available for worms. First they possess a system for mmBCFA biosynthesis that includes two FA elongation enzymes, ELO-5 and ELO-6, which are regulated at least in part by the nematode homolog of SREBP-1c (lpd-1). Second, worms may obtain mmBCFA from their diet (bacteria). Therefore, C. elegans is able to produce, activate, transport, and utilize mmBCFA and is vitally dependent on this system.

The level of C15/C17ISO in eggs appears to be critical for growth and development as animals depleted of C15/C17ISO completely arrest at the L1 stage. The uniformity and reversibility of the arrest would be consistent with a regulatory role for these mmBCFA or more complex lipid molecules containing them on growth and development. However, it cannot be ruled out that the arrest is due to the failure of a metabolic or structural function that is essential for growth and development at the first larval stage. In addition, C15/C17ISO may directly or indirectly regulate genes involved in FA homeostasis. Consistent with this, their deficiency triggers a large alteration in gene expression that may reflect a complex feedback mechanism. Among the potentially responsive genes are transcription factors and metabolic genes.

Ubiquitous and unattended mmBCFAs come forth as physiologically important molecules that regulate essential functions in eukaryotes. Subjects that can be further investigated given the data and description provided herein and related to mmBCFAs include the identification of the other components of the mmBCFA biosynthetic machinery, the components of their transport system, mechanisms by which an organism measures the mmBCFA level, the signaling pathways involved in the mmBCFA responses, mechanisms by which mmBCFA exert their physiological function, whether mmBCFA act alone or as parts of more complex lipids, how mmBCFA are synthesized in mammals, and the specific physiological functions of mmBCFA in mammals.

### Example 2

The following example describes a role for acs-1 in embryogenesis.

Functional acs-1, the gene essential for mmBCFA biosynthesis, is required for cytokinesis during early embryogenesis. The inventors have demonstrated that suppression of acs-1 does not affect cell cycle but causes a failure in cellularization resulting in multinucleated blasotmers polyploidy) and eventually in embryonic lethality (data not shown). This defect may take place as early as at the first cell division. It does not affect polarity and polar body extrusion. This phenotype is doze-dependent; the weaker suppression the more full cell cycles with proper cellularization occur and the more embryos escape lethality.

The embryonic lethality caused by suppression of acs-1 can be partially rescued by temperature sensitive allele of zen-4 encoding homolog of mammalian kinesin-like protein-1. During cell divisions in the C. elegans embryo ZEN-4 is localized to the cleavage furrow (Severson et al., 2000). A suppression of zen-4, itself, results in embryonic phenotype similar to the one observed under the acs-1 suppression (Severson et al., 2000). In the inventors' model, ZEN-4 binds directly or indirectly to the product of the ACS-1 enzymatic activity that may be situated on the cell membrane. Each one is necessary to complete the cleavage. Conceivably, the temperature sensitive allele of zen-4 may bind to cell membrane in the acs-1 independent manner and therefore overcome the acs-1-associated embryonic lethality.

### Example 3

The following example describes a role for acs-1 in protective layer formation.

Reference in this and other examples to acs-1(RNAi)+C13ISO refers to acs-1(RNAi) animals maintained on bacterial lawn supplemented with C13ISO.

A suppression of acs-1 affects formation of eggshell and adult cuticle in C. elegans. It determines the architecture of cuticle and its physical properties (osmotic resistance and withstanding a mechanical pressure) (data not shown).

Experiments by the inventors show that asc-1(RNAi)+C13ISO embryos are osmotic sensitive and burst out in hypotonic solutions. In these embryos the inner layer of eggshell is absent (data not shown). In contrast this layer is doubled in pod-1 mutants that are also sensitive to low salt concentration (Rappleye et al., 1999). This suggests that the dark inner layer is not responsible for osmotic resistance. Unlike acs-1(RNAi)+C13ISO, the pod-1 embryos do not explode in hypotonic environment but expand in volume inside an intact eggs (Rappleye et al., 1999). This indicates that the inner dark layer might be responsible for mechanical durability of an eggshell.

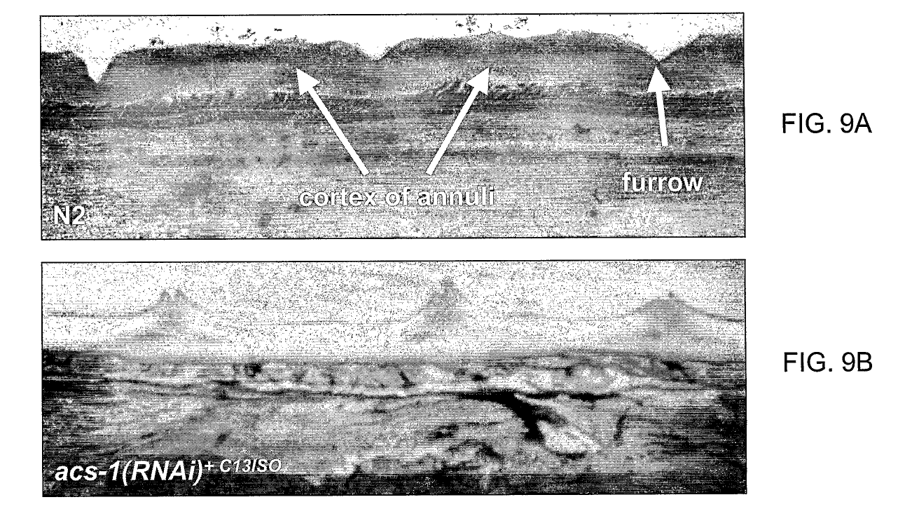

Although a cuticle of adult acs-1(RNAi)+C13ISO is capable of withstanding continuous deformations without rupture and relaxation necessary for smooth locomotion, it is not resistant to hypotonic solutions, in contrast to a cuticle of wild type adult. Electron microscopy reveals a prominent defect in the cuticle architecture (data not shown). Since a structure of the collagen-19 fibers comprising the affected cortical layer of the cuticle seems to be normal, highlighting annuli and furrows as in wild type (col-19::GFP expression data, not shown), the problem may lay in the supporting structure that when absent may cause a collapse of the annuli (FIGS. 9A and 9B). FIGS. 9A and 9B show the abnormal architecture of the adult's cuticle in acs-1(RNAi)+C31ISO. Specifically, FIG. 9A shows an electron micrograph of a wild type adult cuticle (the annuli and furrows are indicated by arrows). FIG. 9B shows an image of the adult acs-1(RNAi)+C13ISO cuticle which is dramatically different.

### Example 4

The following example describes the role of mmBCFA and the DAF pathway in food sensation and insulin signaling.

In this experiment, a suppression of acs-1 combined with temperature sensitive daf-2(e1370) mutation (daf-2 encodes insulin receptor) causes molting/shedding defect in transition between the L4 and adult stages (data not shown). None of the mutations, acs-1 or daf-2, alone displays this phenotype. Therefore, there is a genetic interaction between acs-1, essential for mmBCFA synthesis, and DAF insulin/TGF beta pathway possibly up-stream of DAF-9 (cytochrome P450) in regulation of molting.

In addition, the deficiency of mmBCFA which is caused by suppression of asc-1 or elo-5, essential for mmBCFA biosynthesis, activates expression of pnk-1 and sod-3, two targets of DAF-16 (data not shown).

In another experiment, a deficiency of mmBCFA is shown to stimulate nuclear translocation of DAF-16 (data not shown).

In addition, elo-5 is down-regulated in K01G5.1(RNAi). K01G5.1 encodes a predicted transcription factor that binds to DAF-16 in two hybrid system (data not shown).

L1 arrest caused by mmBCFA deficiency precedes (in developmental scale) the L1 arrest in mid-L1 stage caused by starvation. These early L1 animals are morphologically distinct from L1 animals arrested upon starvation. They have prominently outlined cells instead of smooth tissue-like structures and they are shorter or more compact (data not shown). When fed with the C17ISO supplements on the plates without bacteria, these larvae are able to elongate and proceed to the mid-L1 stage. In this form they can be rescued to normal growth and development by feeding with bacteria. If left without C17ISO supplements but in the presence of bacterial food, they remain at the early stage. Microscope evaluation along with Nile Blue staining revealed that the larvae pump bacterial cells in and that their gut lumen is open. Digesting food generates a “food” signal that initiates growth and development in wild type larvae. Apparently, the mmBCFA deficient larvae are able to feed on bacteria, but unable to process the food signaling without C17ISO. Therefore, C17ISO appears to serve as a part of the food signal processing system.

Neither early nor mid-L1 animals are competent for dauer formation. As the inventors showed above, the DAF-2/DAF-16 pathway is, however, activated in the mmBCFA deficient L1.

These data indicate that in L1, mmBCFA interferes with insulin/DAF pathway not related to dauer formation.

Deficiency of mmBCFA prevents daf-2(e1370), a mutant that forms dauers at 20C and 25C, from proper transition into dauer. The dauer formation in a stronger mutant daf-2(m41) and in pdk-1(sa680) is also affected.

The functions of DAF-2 and PDK-1 encoded by daf-2(e1370) and pdk-1(sa680) correspondingly are sensitive to lipid environment, e.g., to fatty acid composition. A moderate increase in the membrane fluidity promotes reproductive growth, suppressing dauer formation in these mutants. This effect (previously not observed) is related to the nature of mutations that may cause conformational changes in the encoded proteins, both membrane-bound.

### Example 5

The following examples describes the correlation between mmBCFA and growth and specifically, how exogenous C13ISO regulates growth rates and maturation in C. elegans.

As shown above, C13ISO supplementation can rescue the elo-5(gk208) larval lethal phenotype to apparently normal and fertile. The correlation between amounts of the supplement and rescue ability is nonlinear. While there is no rescue from elo-5(gk208) phenotype in a range 0-0.5 mM, at concentration above 0.75 mM 100% of animals reach adulthood and have viable progeny. The growth and maturation rate in these cases are equal to the growth and maturation of N2 on the similarly supplemented plates. At concentrations of 0.75 mM and 1 mM, C13ISO-supplemented N2 and elo-5(gk208) grow as fast as N2 without supplements. However, on the second day of adulthood, the elo-5(gk208) animals fail to lay eggs and die a few days later because of the bag-of-worms phenotype.

The further increase in concentration (to 2.5-10 mM) results in a slowing at the L4 stage accompanied by a delay in the adult maturation. Both N2 and elo-5(gk208) fed with 10 mM C13ISO remain at L4 or young adults stages on forth day after hatching, whereas N2 growing without supplements become gravid adults actively laying eggs. The developmental delay does not seem to be harmful. To the contrary, the animals look healthy, continue to actively lay eggs when control worms slow, and maintain wild type brood size (data not shown). There is no difference in this respect between elo-5(gk208) and N2 supplemented with high concentrations of C13ISO.

The uptake of dietary C13ISO does not change the total FA composition as determined by GC. There are still missing peaks for C15ISO and C17ISO in elo-5(gk208) and no difference in FA content between N2 supplemented with C13ISO and not supplemented. This suggests that C13ISO conveys a delay in maturation and prolonged egg-laying period, and healthy appearance apart from its function as a precursor of longer mmBCFA.

The feature of C13ISO to modulate growth rate between L4 and adult stage is shared by C15ISO and C17ISO, but not by saturated and monounsaturated FA of the 16-18 carbon backbone. It is not related to gross changes in FA composition of total lipids.

### Example 6

The following example shows that the biosynthesis of mmBCFA is tightly linked to protein uptake.

mmBCFA of the ISO-series are products of leucine degradation (Oku and Kaneda, 1988; Kniazeva et al., 2004). Therefore, their levels may be sensitive to protein-based nutrients. Uptake of oligopeptides in all organisms is mediated by a family of proton-coupled peptide transporters (Terada et al., 2004). In C. elegans, the pep-2 gene (also known as opt-2) encodes a oligopeptide transporter that was proposed to be the ortholog of human PEPT1, and it has been shown to be essential for uptake of di-/tripeptides (Nehrke et al., 2003; Meissner et al., 2004). RNAi of pep-2 results in slow postembryonic development and reduced fat content (Nehrke et al., 2003; Meissner et al., 2004; Ashrafi et al., 2003).

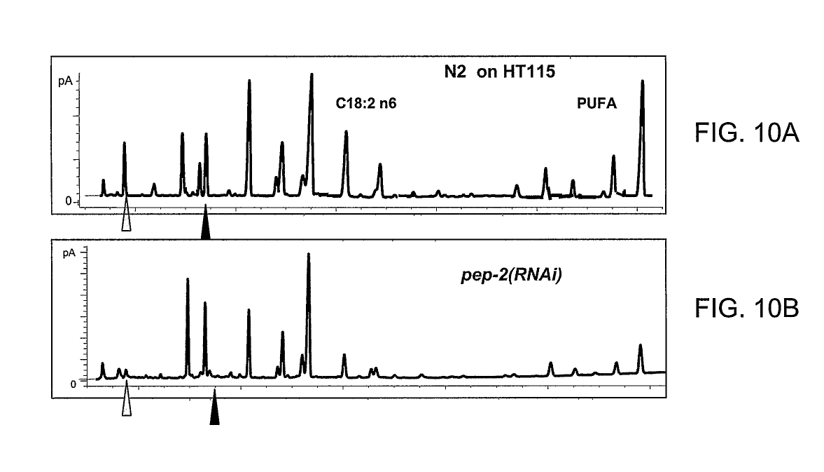

The inventors detected dramatic changes in FA composition of total lipids obtained from mixed population of N2 as compared to pep-2(RNAi) (FIGS. 10A and 10B). FIGS. 10A and 10B compare the fatty acid (FA) composition in wild type animals (FIG. 10A) and pep-2(RNAi) (FIG. 10B) with affected absorption of exogenous peptides. The most dramatic changes are observed in the fractions of C15ISO (gray triangle) and C17ISO (black triangle). The levels of mmBCFA are substantially decreased. Especially affected is a fraction of mmBCFA; the levels of both C15ISO and C17ISO are significantly decreased so that the GC profile of pep-2(RNAi) resembles GC profiles of elo-5(gk208), acs-1(RNAi), pnk-1(RNAi), and lpd-1(RNAi) animals that were described in the Examples above.

Thus, abnormal absorption of oligopeptides causes a selective suppression of the mmBCFA production.

The decrease in mmBCFA levels in pep-2(RNAi) animals could be solely explained by deficiency of leucine as a precursor of mmBCFA. However, the inventors have found that in addition to it, there is an alteration of the mmBCFA production in pep-2(RNAi) at the transcriptional level. The inventors analyzed GFP expression of reporter constructs corresponding to known proteins involved in mmBCFA biosynthesis: elo-5Prom::GFP, acs-1Prom::GFP, pnk-1Prom::GFP, and lpd-1Prom::GFP on the pep-2(RNAi) background. While no significant changes in the expression of acs-1Prom::GFP, pnk-1Prom::GFP, and lpd-1Prom::GFP are observed, a reduction of the level of elo-5Prom::GFP in pep-2(RNAi) animals is significant (data not shown).

Thus, a decrease in mmBCFA in pep-2(RNAi) reflects not only a lessening of the substrate availability; it also indicates an active and selective process of transcriptional suppression of the mmBCFA elongation gene, elo-5.

Since the pep-2(RNAi) animals are viable and fertile; they might have sufficient amino acids from affected peptide absorption and amino acid turnover for continuous protein biosynthesis. The transcriptional down-regulation of mmBCFA biosynthetic enzyme may serve a role to protect the pool of amino acids from depleting the leucine that is a precursor of mmBCFA.

The inventors have therefore established that biosynthesis of mmBCFA is sensitive to dietary protein up-take and, therefore, appears to be involved in food signaling.

### Example 7

The following example shows that elo-S may be a down-stream target of TOR.

Since amino acid availability regulates the TOR signaling pathway in mammals (reviewed in Yoshizawa et al., 2004) and pep-2 interacts with the C. elegans TOR (encoded by the let-363 gene) signaling (Meissner et al., 2004), it is possible that the transcriptional down-regulation of elo-5 on the pep-2 RNAi background may be the result of inhibition of the TOR pathway. The inventors tested effect of inhibiting the ceTOR pathway on the expression of elo-5 and found that elo-5Prom::GFP expression is significantly lower when TOR signaling is affected either by RNAi suppression of let-363 or by the suppression of other components of TOR-pathway including RheB, GTPase that activates TOR encoded by F54C8.8 (Stocker et al., 2003) and the elongation initiation factor E1F4G, a target of TOR encoded by M110.4 (Berset et al., 1998) (data not shown). No differences are observed in the expression of acs-1Prom::GFP, pnk-1Prom::GFP, and lpd-1Prom::GFP.

The RNAi suppression of some other genes involved in protein biosynthesis, possibly apart from TOR pathway, namely, eIF-4E, eIF-1A, small ribosomal subunit, 28S ribosomal subunit, eIF-5A, and phenylalanine t-RNA synthetase, does not cause down-regulation of elo-5Prom::GFP. Therefore, transcriptional control over elo-5 related to the protein malnutrition is likely mediated through TOR pathway. These data indicate a link between food signaling and mmBCFA metabolism.

### Example 8

The following example shows that exogenous mmBCFA affects the expression of pantothenate kinase gene that is essential for CoA biosynthesis.

Exogenous FAs require an activation by esterification to CoA in order to be partitioned for various metabolic and signaling pathways (Coleman et al., 2002). The reaction is carried out by a number of acyl-CoA ligases. There are two sources of CoA, the recycled form and a form that is synthesized de novo. Pantothenate kinase is an essential enzyme in the de novo biosynthesis. It is encoded by pnk-1 in C. elegans. The inventors have previously shown that deficiency of mmBCFA causes up-regulation of pnk-1 (Kniazeva et al., 2004), whereas down-regulation of pnk-1 results in decreased mmBCFA biosynthesis.

In addition, the inventors have found that high levels of mmBCFA (10 mM) added as dietary supplements to the bacterial food actually down-regulate expression of pnk-1 (data not shown). This indicates a negative feedback control of the pnk-expression by mmBCFA and establishes a link between CoA metabolism and mmBCFA.

Coenzyme A is essential for the initiation of Krebs cycle and ultimately for the body's energy production. The decrease in pnk-1 expression in response to high levels of dietary mmBCFA may account for the slow energy production and consequently for slow growth rates discussed above.

The responsiveness of the mmBCFA metabolism to exogenous protein up-take and its ability to influence CoA biosynthesis suggest a role for mmBCFA in coordination of the food signaling and energy expenditure.

### Example 9

The following example demonstrates that human cells have a system capable of elongation of exogenous short chain mmBCFA.

Cultured mammalian cells including human HEK-23 (embryonic kidney), SY-5Y (neuroblastoma), RIN-M5F (pancreatic beta cells), and mouse C2C12 (myoblasts) are able to elongate C13ISO, the shorter mmBCFA, into C15ISO and C17ISO in vitro. These cell lines may therefore be used to study physiological effect of mmBCFAs as well as to identify mmBCFA-related enzymes and mmBCFA signaling system in mammals.

Each reference cited below and elsewhere herein is incorporated herein by reference in its entirety.

### REFERENCES

Ashrafi, K. et al. Genome-wide RNAi analysis of Caenorhabditis elegans fat regulatory genes. Nature 421, 268-72 (2003).
Berset, C., Trachsel, H., Altmann, M., Institute for, B. & Molecular Biology, U.o.B.B.B.S. The TOR (target of rapamycin) signal transduction pathway regulates the stability of translation initiation factor eIF4G in the yeast Saccharomyces cerevisiae. Proceedings of the National Academy of Sciences of the United States of America. 95(8), 4264-9 (1998).
Coleman, R. A., Lewin, T. M., Van Horn, C. G. & Gonzalez-Baro, M. R. Do long-chain acyl-CoA synthetases regulate fatty acid entry into synthetic versus degradative pathways? Journal of Nutrition 132, 2123-6 (2002).
Hradec, J. and P. Dufek (1994). “Determination of cholesteryl 14-methylhexadecanoate in blood serum by reversed-phase high-performance liquid chromatography.” J Chromatogr B Biomed Appl 1660(2): 386-9.
Jones, L. N., D. J. Peet, et al. (1996). “Hairs from patients with maple syrup urine disease show a structural defect in the fiber cuticle.” Journal of Investigative Dermatology 106(3): 461-4.
Jones, L. N. and D. E. Rivett (1997). “The role of 18-methyleicosanoic acid in the structure and formation of mammalian hair fibres.” Micron 28(6): 469-85.
Kniazeva, M., M. Sieber, et al. (2003). “Suppression of the ELO-2 FA Elongation Activity Results in Alterations of the Fatty Acid Composition and Multiple Physiological Defects, Including Abnormal Ultradian Rhythms, in Caenorhabditis elegans.” Genetics 163(1): 159-69.
Kniazeva, M. et al. Monomethyl Branched-Chain Fatty Acids Play an Essential Role in Caenorhabditis elegans Development. PLoS biology. 2(9), E257 (2004).
McKay R. M., J. P. McKay, et al. (2003). “C elegans: a model for exploring the genetics of fat storage.” Developmental Cell 4(1): 131-42.
Meissner, B., Boll, M., Daniel, H., Baumeister, R. & Acolph-Butenandt-Institute Molecular Neurogenetics, L.-M.-U.o.M.D.M.G. Deletion of the intestinal peptide transporter affects insulin and TOR signaling in Caenorhabditis elegans. The Journal of biological chemistry. 279(35), 36739-45 (2004).
Miquel, M. and J. Browse (1992). “Arabidopsis mutants deficient in polyunsaturated fatty acid synthesis. Biochemical and genetic characterization of a plant oleoyl-phosphatidylcholine desaturase.” J Biol Chem 267(3): 1502-9.
Nehrke, K. & Gastroenterology Unit, D.o.M.U.o.R.M.C.R.N.Y.U.S.A.k.n.u.r.e. A reduction in intestinal cell pHi due to loss of the Caenorhabditis elegans Na+/H+ exchanger NHX-2 increases life span. The Journal of biological chemistry. 278(45), 44657-66 (2003).
Oka, H. and T. Kaneda (1988). “Biosynthesis of branched-chain fatty acids in Bacillus subtilis. A decarboxylase is essential for branched-chain fatty acid synthetase.” Journal of Biological Chemistry 263(34): 18386-96.
Ramsey, R. B., T. Scott, et al. (1977). “Fatty acid composition of myelin isolated from the brain of a patient with cellular deficiency of co-enzyme forms of vitamin B12.” J Neurol Sci 34(2): 221-32.
Rappleye, C. A. et al. The coronin-like protein POD-1 is required for anterior-posterior axis formation and cellular architecture in the nematode caenorhabditis elegans. Genes & development. 13(21), 2838-51 (1999).
Rilfors, L., A. Wieslander, et al. (1978). “Lipid and protein composition of membranes of Bacillus megaterium variants in the temperature range 5 to 70 degrees C.” J Bacteriol 135(3): 1043-52.
Smith, E. J. and T. Kaneda (1980). “Relationship of primer specificity of fatty acid de novo synthetase to fatty acid composition in 10 species of bacteria and yeasts.” Canadian Journal of Microbiology 26(8): 893-8.
Severson, A. F. et al. The aurora-related kinase AIR-2 recruits ZEN-4/CeMKLP1 to the mitotic spindle at metaphase and is required for cytokinesis. Current biology: CB. 10(19), 1162-71 (2000).
Stocker, H. et al. Rheb is an essential regulator of S6K in controlling cell growth in Drosophila. Nature cell biology. 5(6), 559-65 (2003).
Terada, T., Inui, K. & Department of Pharmacy, K.U.H.F.o.M.K.U.S.-k.K.J. Peptide transporters: structure, function, regulation and application for drug delivery. Current drug metabolism. 5(1), 85-94 (2004).
Tuhackova, Z. and J. Hradec (1985). “The role of cholesteryl 14-methylhexadecanoate in the function of eukaryotic peptide elongation factor 1.” European Journal of Biochemistry 146(2): 365-70.
van de Vossenberg, J. L., A. J. Driessen, et al. (1999). “Homeostasis of the membrane proton permeability in Bacillus subtilis grown at different temperatures.” Biochim Biophys Acta 1419(1): 97-104.
Yang, Z., S. Liu, et al. (2000). “Induction of apoptotic cell death and in vivo growth inhibition of human cancer cells by a saturated branched-chain fatty acid, 13-methyltetradecanoic acid.” Cancer Research 60(3): 505-9.
Yoshizawa, F. & Department of Animal Science, U.U.U.T.J.f.c.u.-u.aj. Regulation of protein synthesis by branched-chain amino acids in vivo. Biochemical and biophysical research communications. 313(2), 417-22 (2004).

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. A non-human animal model for studying metabolism or homeostasis of mono-methyl branched-chain fatty acids (mmBCFA), the regulation of growth, the regulation of development or the regulation of reproduction in eukaryotic organisms, wherein the non-human animal model has been modified to delete or inactivate a protein or functional homologue thereof, the protein selected from the group consisting of: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (RuvB-like) (SEQ ID NO:20), pantothenate kinase (PNK-1) (SEQ ID NO:22), branched-chain a-keto-acid dehydrogenase (BCKAD) a subunit (SEQ ID NO:24), BCKAD pyruvate dehydrogenase subunit (SEQ ID NO:38), and oligopeptide transporter PEP-2 (PEP-2) (SEQ ID NO:26).

2. The non-human animal model of claim 1, wherein the non-human animal is produced using RNAi targeted to RNA encoding the protein or homologue thereof.

3. The non-human animal model of claim 1, wherein the animal is C. elegans.

4. An isolated cell for evaluating the biosynthesis and function of mono-methyl branched-chain fatty acid (mmBCFA) in vitro, comprising a eukaryotic cell that produces mmBCFA, wherein the cell has a modification resulting in the deletion or inactivation of at least one protein or functional homologue thereof, the protein selected from the group consisting of: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (RuvB-like) (SEQ ID NO:20), pantothenate kinase (PNK-1) (SEQ ID NO:22), branched-chain a-keto-acid dehydrogenase (BCKAD) a subunit (SEQ ID NO:24), BCKAD pyruvate dehydrogenase subunit (SEQ ID NO:38), and oligopeptide transporter PEP-2 (PEP-2) (SEQ ID NO:26).

5. A method to identify compounds that regulate the biosynthesis or function of mono-methyl branched-chain fatty acids (mmBCFA) in a eukaryotic organism, comprising identifying a compound that regulates the expression or biological activity of a C. elegans protein or a eukaryotic homologue thereof, the protein selected from the group consisting of: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (SEQ ID NO:20), pantothenate kinase (PNK-1) (SEQ ID NO:22), branched-chain a-keto-acid dehydrogenase (BCKAD) a subunit (SEQ ID NO:24), BCKAD pyruvate dehydrogenase subunit (SEQ ID NO:38), and oligopeptide transporter PEP-2 (PEP-2) (SEQ ID NO:26).

6. The method of claim 5, wherein the identification of compounds that increase the expression or biological activity of the protein or homologue thereof are selected as compounds that increase the biosynthesis or function of mmBCFA, and wherein the identification of compounds that decrease the expression or biological activity of the protein or homologue thereof are selected as compounds that decrease the biosynthesis or function of mmBCFA.

7-11. (canceled)

12. The method of claim 5, comprising detecting the ability of a compound to regulate the production of an mmBCFA selected from the group consisting of C15ISO and C17 ISO.

13. (canceled)

14. The method of claim 5, comprising the steps of:
a) contacting a host cell with a putative regulatory compound, wherein the host cell expresses the protein or homologue thereof or a biologically active fragment thereof; and
b) detecting whether the putative regulatory compound inhibits or increases the expression or biological activity of the protein or homologue thereof or biologically active fragment thereof;
wherein a putative regulatory compound that inhibits the expression or biological activity of the protein as compared to in the absence of the compound is selected as a compound for inhibiting mmBCFA biosynthesis or function in a eukaryotic organism; and
wherein a putative regulatory compound that increases the biological activity of the protein as compared to in the absence of the compound is selected as a compound for increasing mmBCFA biosynthesis or function in a eukaryotic organism.

15-18. (canceled)

19. The method of claim 5, comprising the steps of:
a) administering a putative regulatory compound to a non-human animal that has been modified to delete or inactivate a protein or functional homologue thereof, the protein selected from the group consisting of: long chain fatty acid elongation enzyme ELO-5 (SEQ ID NO:10), long chain fatty acid elongase enzyme ELO-6 (SEQ ID NO:12), mmBCFA-specific acetyl-CoA synthetase (ACS-1) (SEQ ID NO:14), LiPid Depleted 1 (LPD-1) (SEQ ID NO:16), nuclear hormone receptor 49 (NHR-49) (SEQ ID NO:18), RuvB-like DNA binding protein (RuvB-like) (SEQ ID NO:20), pantothenate kinase (PNK-1) (SEQ ID NO:22), branched-chain a-keto-acid dehydrogenase (BCKAD) a subunit (SEQ ID NO:24), BCKAD pyruvate dehydrogenase subunit (SEQ ID NO:38), oligopeptide transporter PEP-2 (PEP-2) (SEQ ID NO:26); phosphoinositide-dependent protein kinase 1 (PDK-1) (SEQ ID NO:28); and insulin receptor DAF-2 (DAF-2) (SEQ ID NO:30);
b) detecting a change in the non-human animal in the presence of the compound as compared to in the absence of the compound, the change being selected from the group consisting of:
i) an increase or decrease in the expression or biological activity of the protein or homologue thereof;
ii) an increase or decrease in the amount or type of mmBCFA synthesized by the non-human animal;
iii) a change in the total fatty acid profile of the non-human animal;
iv) an increase or decrease in insulin-signaling in the non-human animal;
v) a change in embryogenesis in the non-human animal or progeny thereof;
vi) a change in the fertility of the non-human animal or progeny thereof;
vii) a change in the viability of progeny of the non-human animal;
viii) an increase or decrease in the growth or development of the non-human animal or progeny thereof; and
ix) a change in a metabolic response to food sensation in the non-human animal; and
c) selecting a compound as a compound that regulates mmBCFA biosynthesis or function if a change is detected in (b) in the presence of the compound as compared to the absence of the compound.

20. (canceled)

21. The method of claim 19, wherein the non-human animal has a modification that results in the deletion or inactivation of a protein or combination of proteins selected from the group consisting of: ELO-5, ACS-1, LPD-1, ACS-1 and ELO-1, RuvB-like protein or homologue thereof, PNK-1 NHR-49, BCKAD and ELO-6.

22-41. (canceled)

42. The method of claim 19, wherein the method further comprises, either before, during or after step (a) or (b), a step of providing an exogenous mmBCFA selected from the group consisting of: C13ISO, C15ISO, C17ISO, C15ante-ISO, C17-anteISO, and a methyl ester thereof, wherein the method further comprises a step of detecting a change in the non-human animal in the presence and absence of the exogenous mmBCFA.

43. A formulation comprising at least one mono-methyl branched-chain fatty acid (mmBCFA) selected from the group consisting of: C13ISO, C15ISO, C17ISO, C15ante-ISO, and C17-anteISO, or a functional derivative of any of the mmBCFA or a methyl ester of any of the mmBCFA, or combinations thereof.

44. (canceled)

45. The formulation of claim 43, wherein the mmBCFA is selected from the group consisting of C17ISO and C17-anteISO, or a methyl ester thereof.

46. (canceled)

47. The formulation of claim 43, further comprising at least one additional dietary agent selected from the group consisting of a vitamin, a mineral, a protein, a carbohydrate, and a lipid.

48-49. (canceled)

50. The formulation of claim 43, further comprising at least one agent for the treatment of a disease or condition, or a symptom thereof, wherein the disease or condition is associated with metabolism, growth, development or reproduction of a eukaryotic organism.

51-52. (canceled)

53. A method to increase mono-methyl branched-chain fatty acid (mmBCFA) in a eukaryotic organism, comprising administering to the organism the formulation of claim 43.

54-58. (canceled)

59. A method to treat a patient with Maple Syrup Urine Disease, comprising administering to a patient with Maple Syrup Urine Disease the formulation of claim 43.

60. A method to regulate or evaluate insulin-signaling in a eukaryotic organism, comprising regulating in the organism the level of at least one mono-methyl branched-chain fatty acid (mmBCFA) selected from the group consisting of: C13ISO, C15ISO, and C17ISO, wherein the step of regulating mmBCFA regulates insulin-signaling, fat storage, or growth and development of the organism, wherein the step of regulating comprises administering to the organism the formulation of claim 43.

61-63. (canceled)


SEQUENCE LISTING (38 sequences)

SEQ ID NO: 1 - DNA, 24 bp, Caenorhabditis elegans
tttaggtcattttttgagtcgcca

SEQ ID NO: 2 - DNA, 26 bp, Caenorhabditis elegans
tagtctggaattttgaaattgaacgg

SEQ ID NO: 3 - DNA, 27 bp, Caenorhabditis elegans
gcccttggaaaccatctacgacgaatc

SEQ ID NO: 4 - DNA, 28 bp, Caenorhabditis elegans
tccgaacagaacgacataagagatttcc

SEQ ID NO: 5 - DNA, 23 bp, Caenorhabditis elegans
cataattactattgcgtcacatg

SEQ ID NO: 6 - DNA, 23 bp, Caenorhabditis elegans
ctcttccaaactggcgatgtcga

SEQ ID NO: 7 - DNA, 24 bp, Caenorhabditis elegans
tcgtacgatcggaccataggctaa

SEQ ID NO: 8 - DNA, 24 bp, Caenorhabditis elegans
ctgatcctctgtagcagcggccct

SEQ ID NO: 9 - DNA, 861 bp, Caenorhabditis elegans
  CDS (1)..(861)
atgtcatcggacgatcgtggcactagaaccttcaagatgatggatcaaattcttggaacaaacttcacttatgaaggtgccaaagaagttgctcgaggccttgaaggtttctcagcaaagcttgccgtcggatatattgccactatttttggactgaaatattatatgaaagaccgaaaagccttcgatctcagtactccattaaacatttggaatggtattctttcgacattcagcttattgggattcttattcacttttcctactttgttatcagttatcagaaaggatggatttagtcacacctattcccatgtctctgagctttacactgacagtacctctggatattggatcttcctttgggttatctcaaagattccggaacttttggatacagtattcattgttcttcgcaagagaccacttattttcatgcactggtaccatcacgcattgaccggttactatgctcttgtctgctaccatgaggatgctgtccatatggtttgggttgtatggatgaattatattattcatgcattcatgtatggatactatcttctgaaatctctgaaagttccaattccaccatcagttgctcaagcaatcaccacatctcaaatggttcaattcgcagttgccattttcgcacaagttcatgtttcctataaacactatgttgagggagttgaaggattagcctactcgttcagaggaacagctatcggatttttcatgcttactacctacttctatctatggattcaattctacaaagagcactatcttaagaatggaggcaaaaagtacaatttggcaaaggatcaggcaaaaactcaaacaaagaaggctaactaa

SEQ ID NO: 10 - protein, 286 aa, Caenorhabditis elegans
MSSDDRGTRTFKMMDQILGTNFTYEGAKEVARGLEGFSAKLAVGYIATIFGLKYYMKDRKAFDLSTPLNIWNGILSTFSLLGFLFTFPTLLSVIRKDGFSHTYSHVSELYTDSTSGYWIFLWVISKIPELLDTVFIVLRKRPLIFMHWYHHALTGYYALVCYHEDAVHMVWVVWMNYIIHAFMYGYYLLKSLKVPIPPSVAQAITTSQMVQFAVAIFAQVHVSYKHYVEGVEGLAYSFRGTAIGFFMLTTYFYLWIQFYKEHYLKNGGKKYNLAKDQAKTQTKKAN

SEQ ID NO: 11 - DNA, 825 bp, Caenorhabditis elegans
  CDS (1)..(825)
atgccacagggagaagtctcattctttgaggtgctgacaactgctccattcagtcatgagctctcaaaaaagcatattgcacagactcagtatgctgctttctggatctcaatggcatatgttgtcgttatttttgggctcaaggctgtcatgacaaaccgaaaaccatttgatctcacgggaccactgaatctctggaatgcgggtcttgctattttctcaactctcggatcacttgccactacatttggacttctccacgagttcttcagccgtggatttttcgaatcttacattcacatcggagacttttataatggactttctggaatgttcacatggcttttcgttctctcaaaagttgctgaattcggagatacactttttattattcttcgtaaaaagccattgatgttccttcattggtatcatcatgtgcttacaatgaattatgcttttatgtcatttgaagctaatttgggatttaatacttggattacatggatgaatttctcagttcactcaattatgtatggatattatatgcttcgttcttttggtgtcaaggttccagcatggattgccaagaatattacaacaatgcaaattcttcaattcgttattactcatttcattcttttccacgttggatatttggcagttactggacaatctgttgactcaactccaggatattattggttctgccttctcatggaaatctcttatgtcgttctgttcggaaacttctactatcaatcatacatcaagggaggtggcaagaagtttaatgcagagaagaagactgaaaagaaaattgaataa

SEQ ID NO: 12 - protein, 274 aa, Caenorhabditis elegans
MPQGEVSFFEVLTTAPFSHELSKKHIAQTQYAAFWISMAYVVVIFGLKAVMTNRKPFDLTGPLNLWNAGLAIFSTLGSLATTFGLLHEFFSRGFFESYIHIGDFYNGLSGMFTWLFVLSKVAEFGDTLFIILRKKPLMFLHWYHHVLTMNYAFMSFEANLGFNTWITWMNFSVHSIMYGYYMLRSFGVKVPAWIAKNITTMQILQFVITHFILFHVGYLAVTGQSVDSTPGYYWFCLLMEISYVVLFGNFYYQSYIKGGGKKFNAEKKTEKKIE

SEQ ID NO: 13 - DNA, 1872 bp, Caenorhabditis elegans
  CDS (1)..(1872)
atgtcacaagtggccgcaatggaccttcgtgtacttacacagttcgacatcgccagtttggaagaggaccgcaaaaagatgttgtacgaggagccgatctctttggaagaagccgctctgaacgccaacgacgtgatggtggcgccgagtcgaaagtcgtatgtgcacggctgttcgactgttcctttgctttttgaaactgttggagatcgacttcgatcagcagttgaccaggttccagataaggaatttttgattttcaaaagagaaggaatcaggaaaacttattcgcaagtcgccacagatgcagaaaacctggcttgcgggctcctccacttgggtttgaaaaaaggagatcgtattggaatttgggggccaaacacatacgagtggaccacaacacagtttgccagtgctcttgccggaatggttctagtcaacataaacccatcatatcaatcagaagaacttcgctatgctattgaaaaagtaggaatcagagcccttatcacaccacctggattcaagaagtcaaattattatcagagcatcaaggatattctgccagaagttacattgaaagaaccgggaaagagtggaatcacatcgagaaatttcacatgtttccaacacttgatcatgtttgacgaggaagataagatctatccaggagcctggaaatacacagatgtaatgaaaatgggaacagaagaagacagacaccacctctcaaagatcgagagggaaactcaaccagatgactcactgaacattcaatacacaagtggaacaactggacagcctaaaggagcaactctcactcatcacaatgttctcaataatgcattttttgttggtctccgtgctggatatagtgaaaagaagacaattatctgcattccaaatccactttatcactgttttggatgtgtcatgggagttctcgccgcactcacacaccttcaaacttgcgtattccctgctccatcattcgatgctctcgctgctcttcaagctattcacgaggaaaaatgcacagctctttatggaacaccaacaatgtttattgatatgattaatcatcccgagtatgcaaactataactatgattcaattagaagtggattcattgctggagctccgtgtccgataacactttgccgccggttagtacaagatatgcatatgactgacatgcaagtatgctatggaactactgaaacatctccagtatcttttatgtcaacacgtgatgatccaccagagcaaaggatcaaatcagtcggacatattatggatcacttggaagctgcaattgtagacaagagaaactgcattgttccacgtggcgtaaaaggagaagttattgtccgtggatactcagttatgcggtgctattggaacagtgaagagcagacaaagaaggaaatcactcaagacagatggtatcatactggagatattgctgttatgcatgataatggaaccatctctattgttggaagatcgaaagatatgattgttcgtggaggagagaatatttatcctaccgaagtagaacaatttttattcaagcatcagtcagtagaagatgttcatattgtcggagtaccagatgaacgattcggtgaggtggtctgcgcctgggtcagattgcacgaaagcgctgaaggaaaaacgactgaagaggatattaaggcttggtgcaagggaaaaattgctcatttcaaaattcctcgctacatcctcttcaaaaaggagtacgaattcccgctgacagtgactggaaaagtgaaaaagtttgaaatccgcgaaatgtcaaagattgaactgggtctccagcaagtcgtctctcatttctccgagctttaa

SEQ ID NO: 14 - protein, 623 aa, Caenorhabditis elegans
MSQVAAMDLRVLTQFDIASLEEDRKKMLYEEPISLEEAALNANDVMVAPSRKSYVHGCSTVPLLFETVGDRLRSAVDQVPDKEFLIFKREGIRKTYSQVATDAENLACGLLHLGLKKGDRIGIWGPNTYEWTTTQFASALAGMVLVNINPSYQSEELRYAIEKVGIRALITPPGFKKSNYYQSIKDILPEVTLKEPGKSGITSRNFTCFQHLIMFDEEDKIYPGAWKYTDVMKMGTEEDRHHLSKIERETQPDDSLNIQYTSGTTGQPKGATLTHHNVLNNAFFVGLRAGYSEKKTIICIPNPLYHCFGCVMGVLAALTHLQTCVFPAPSFDALAALQAIHEEKCTALYGTPTMFIDMINHPEYANYNYDSIRSGFIAGAPCPITLCRRLVQDMHMTDMQVCYGTTETSPVSFMSTRDDPPEQRIKSVGHIMDHLEAAIVDKRNCIVPRGVKGEVIVRGYSVMRCYWNSEEQTKKEITQDRWYHTGDIAVMHDNGTISIVGRSKDMIVRGGENIYPTEVEQFLFKHQSVEDVHIVGVPDERFGEVVCAWVRLHESAEGKTTEEDIKAWCKGKIAHFKIPRYILFKKEYEFPLTVTGKVKKFEIREMSKIELGLQQVVSHFSEL

SEQ ID NO: 15 - DNA, 3342 bp, Caenorhabditis elegans
  CDS (1)..(3342)
atgaacgaagaattcgagggagacgtccctatgtcggatccgtttctctcattggtcacaaaattggatgatattgcgccatttccaaataacgacccgctcgattttgacatggagcacaactggcaagagcccggaccatcacaacaaccggatccatcaattcccggaaatcaacacagtccgccacaggaatattatgatattgatggtcaacgagacgtaagcaccttacactccctgctcaaccacaacaacgacgacttcttctcaatgcgattttccccgccaaactttgatctcggcggaggccgtggaccttctctagccgccacccaacaattatctggagaaggtcctgcaagtatgcttaaccccttacaaacatctccaccaagtggaggttaccccccggcagatgcctacagacctctatcacttgctcaacaactcgccgcgccagcgatgactccacatcaggcagcgtcgctttttgttaatactaatggaattgatcaaaagaatttcactcatgcaatgctatctccaccacaccatacctcaatgactcctcaaccatatacagaagcaatggaacatatcaacgggtacatgtctccatacgaccaagctcaaggcccatcaggaccatcatattactcacaacaccatcaatctccaccacctcatcaccaccatcaccacccgatgccaaaaatccatgagaaccctgaacaagtggcatctccatcgattgaagatgctccagagacgaaaccaactcatttggttgaaccacaaagtccaaaaagcccgcagaatatgaaagaggagcttcttcggttactagttaacatgtctccgagtgaagttgaacggttaaagaataaaaaatcaggagcatgttcagcgacgaatgggccatcgaggagtaaggagaaggcggcgaagattgtgattcaggagacagcggaaggggatgaagatgaggatgatgaggatagtgattccggggagactatgtctcagggaactactattattgttcgaagaccaaaaaccgagcgtcgtacggcacacaatctcatcgaaaagaagtatagatgctcaataaatgatcgaattcaacagctgaaagtacttttgtgtggggatgaagctaagctttcaaaatcggcaacactacgacgggctattgaacatatcgaggaggttgaacacgagaatcaggtgttgaagcatcatgttgaacaaatgagaaagacactgcagaataatcgattaccgtacccggaaccaattcaatacactgaatactctgcccgatcacccgtcgaatcatctccttctccacctagaaatgagagaaaacgatcacgaatgagcacaacgactcctatgaagaatggaactagagatggatcttcgaaagttaccctttttgcgatgctcctagcagttctgatttttaatccgattggattgctcgctggaagtgcgatattctcaaaagccgctgcagaagctccgattgcctccccgttcgagcatggaagagtgattgatgacccggatggaactagcactcggacgcttttctgggaagggagtatcatcaatatgagctatgtctgggtgttcaacatcttaatgatcatatatgtggttgtcaaactgctgatccatggtgaccctgttcaagacttcatgtccgtttcatggcagacttttgtgacgactcgagagaaggcgagagccgagttgaactctggaaatttgaaagatgctcagagaaagttctgcgagtgtcttgcaacgttggatcgatcgcttccatcaccgggggttgattcggtgttttcggttggctgggaatgcgttcgacatcttttgaattggttgtggatcgggagatacatcgcaagaaggcgcaggtccaccacgaagcctgtctcagtcgtttgtaggagtcatgcgcagactgcagttctctatcatgaaattcatcagctccatctaatgggtatcactggaaacttcgaagacacctatgaaccatccgccctaacgggcctcttcatgtccctctgtgcagtaaaccttgctgaagctgccggagcatcaaacgacggacttccacgcgccgtcatggctcagatctacatttctgcatccatccaatgccgtttggctcttccgaacctactcgcaccattcttctcgggatactttttacgaagagctcgaaggcacgtgcgtcgagctccggagcactcggtgtcccatttgttatggatcttccatccagcgacaagaaagttcatgtcagatgcgaaaaggttggagcatgtgttgagctcgaagcagaagcagttgagatttgggtcttttgtggaagatgagcaattatccccacttgctcgaatccgaacaacgctgaaagtgtacctactctccaaacttgtacaggaacttgtcggtggtgacgagatctttacaaaaaatgtggaacgcatcctaaatgacaatgaccgtctcgatgatgaagtagacgtggttgatgtttcaagacttttggtgacaatttcaacgcagtgcgctgccattttgactaatgagaaggatgagtcagcgaaattcggaacctggatctctcgaaacggagatgcttgttgcacatggtggacgcacgttctgacatgtggaatctattggaggagtaacaagaatgagctggcacggcaacactattcactgatcaggaactgtccgccgaagattttgacagacaatctgggtttggcggttggccacgcgttgtgtgctcgcaagatttgcatagatgaccgagattccccgaaagtcagtcaatacgtgtgcattcacacaaagaagtcgctcgaatccctccgactattctccacatcatcgcgagcatcaggtgtggtgtctggaattcaggaaggtacacgccgaatggcctacgaatggattatgaactcgctgctcgacgcgtggcgttccaatctattcgcatcgaaaccctactggacacaaagcttcaagggacaatccacgtttagtacgctttatcaagaggcgtataatcattatgcgattattaatgggacaaggggagattgttggagactatttgtctacgagctcacgtgccgaatgctcaacggagccaacccacaagccacgtggtcaggcgtccgacgcgttcgatctacaaaaatggacgcggtccgaggaagagtgagcatgcgacgctcggctcaaccggacgcatttcatcttcatacactggttaaactacatacttctatggatctttga

SEQ ID NO: 16 - protein, 1113 aa, Caenorhabditis elegans
MNEEFEGDVPMSDPFLSLVTKLDDIAPFPNNDPLDFDMEHNWQEPGPSQQPDPSIPGNQHSPPQEYYDIDGQRDVSTLHSLLNHNNDDFFSMRFSPPNFDLGGGRGPSLAATQQLSGEGPASMLNPLQTSPPSGGYPPADAYRPLSLAQQLAAPAMTPHQAASLFVNTNGIDQKNFTHAMLSPPHHTSMTPQPYTEAMEHINGYMSPYDQAQGPSGPSYYSQHHQSPPPHHHHHHPMPKIHENPEQVASPSIEDAPETKPTHLVEPQSPKSPQNMKEELLRLLVNMSPSEVERLKNKKSGACSATNGPSRSKEKAAKIVIQETAEGDEDEDDEDSDSGETMSQGTTIIVRRPKTERRTAHNLIEKKYRCSINDRIQQLKVLLCGDEAKLSKSATLRRAIEHIEEVEHENQVLKHHVEQMRKTLQNNRLPYPEPIQYTEYSARSPVESSPSPPRNERKRSRMSTTTPMKNGTRDGSSKVTLFAMLLAVLIFNPIGLLAGSAIFSKAAAEAPIASPFEHGRVIDDPDGTSTRTLFWEGSIINMSYVWVFNILMIIYVVVKLLIHGDPVQDFMSVSWQTFVTTREKARAELNSGNLKDAQRKFCECLATLDRSLPSPGVDSVFSVGWECVRHLLNWLWIGRYIARRRRSTTKPVSVVCRSHAQTAVLYHEIHQLHLMGITGNFEDTYEPSALTGLFMSLCAVNLAEAAGASNDGLPRAVMAQIYISASIQCRLALPNLLAPFFSGYFLRRARRHVRRAPEHSVSHLLWIFHPATRKFMSDAKRLEHVLSSKQKQLRFGSFVEDEQLSPLARIRTTLKVYLLSKLVQELVGGDEIFTKNVERILNDNDRLDDEVDVVDVSRLLVTISTQCAAILTNEKDESAKFGTWISRNGDACCTWWTHVLTCGIYWRSNKNELARQHYSLIRNCPPKILTDNLGLAVGHALCARKICIDDRDSPKVSQYVCIHTKKSLESLRLFSTSSRASGVVSGIQEGTRRMAYEWIMNSLLDAWRSNLFASKPYWTQSFKGQSTFSTLYQEAYNHYAIINGTRGDCWRLFVYELTCRMLNGANPQATWSGVRRVRSTKMDAVRGRVSMRRSAQPDAFHLHTLVKLHTSMDL

SEQ ID NO: 17 - DNA, 1461 bp, Caenorhabditis elegans
  CDS (1)..(1461)
atgaagaatcttcagatgattgtaatcatggacctagtagatcctcttgcggaaccatgtgccgtttgtggtgacaaatcaactggaacccattatggagtcatttcctgcaacgggtgtaagggattcttccgccgaacagttcttcgtgatcagaagttcacttgccgtttcaacaaaagatgtgtgattgacaaaaactttcgatgcgcgtgtcgttattgtcgctttcaaaagtgtgtacaagttggaatgaaacgagaagctattcaattcgaacgtgatcctgtaggttcaccaacatctggagccagtctcaacgggactccattcaaaaaagacagaagccccggatacgagaacggaaacagcaacggtgtcggatcaaacggtatgggacaagagaatatgcgaacagttccacaatccagcagtgtcattgatgctttaatggagatggaggctcgtgtcaatcaagagatgtgtaatcgataccgaagatcgcaaatctttgcaaacggcagtggggggtcgaatggaaatgacacagatattcaacaaggaagtgattctggagcatcggcatttgcaccaccaaaccggccatgcacaaccgaggtagatctgaatgaaatctccaggacaacacttttattgatggttgaatgggcaaaaacaattaatccattcatggatctttcgatggaagataagatcattctactgaaaaactatgcaccacaacatctcattttgatgccagcatttcgaagtcccgatacaactcgagtttgtctcttcaacaatacttatatgacacgtgacaataacactgacctcaatggatttgctgcattcaaaacaagcaatataacacccagagttcttgatgaaattgtctggccaatgcgacaacttcagatgagagaacaagaatttgtatgtttgaaagcacttgcattcttgcacccagaagccaaaggactctcgaatagctcacagattatgattcgtgatgctagaaatcgtgttttgaaagctctttatgcattcattctcgatcagatgccagatgacgcacccacaagatatggaaatattttgttgttggcaccggctctcaaggctctgactcagctgctcattgagaatatgacacttacaaagttcttcggattggcagaggtggattctctgctctccgagttcattctcgacgacatcaatgatcattcgacggctccggtctctttacagcaacatctttcatctccgacaacgttaccgactaatggggtatctccgttaaatccagccggatcagtgggaagtgtttcgtctgtttctggaattacaccaactggaatgctctcagcaactcttgcagctccattggcaattcatcctctccaatcacaagattccattttgaacagtgagcagaataatcatatgctctaa

SEQ ID NO: 18 - protein, 486 aa, Caenorhabditis elegans
MKNLQMIVIMDLVDPLAEPCAVCGDKSTGTHYGVISCNGCKGFFRRTVLRDQKFTCRFNKRCVIDKNFRCACRYCRFQKCVQVGMKREAIQFERDPVGSPTSGASLNGTPFKKDRSPGYENGNSNGVGSNGMGQENMRTVPQSSSVIDALMEMEARVNQEMCNRYRRSQIFANGSGGSNGNDTDIQQGSDSGASAFAPPNRPCTTEVDLNEISRTTLLLMVEWAKTINPFMDLSMEDKIILLKNYAPQHLILMPAFRSPDTTRVCLFNNTYMTRDNNTDLNGFAAFKTSNITPRVLDEIVWPMRQLQMREQEFVCLKALAFLHPEAKGLSNSSQIMIRDARNRVLKALYAFILDQMPDDAPTRYGNILLLAPALKALTQLLIENMTLTKFFGLAEVDSLLSEFILDDINDHSTAPVSLQQHLSSPTTLPTNGVSPLNPAGSVGSVSSVSGITPTGMLSATLAAPLAIHPLQSQDSILNSEQNNHML

SEQ ID NO: 19 - DNA, 1377 bp, Caenorhabditis elegans
  CDS (1)..(1377)
atggatatggaagtcgatgaggcaatatctggtacttcttcttcaagacttgcaccaatcgaagaagtaaaaccaactccaaagcaaattaaacgcattgcggctcacagtcacgtcaagggactcggaattgacacagaaacacaagaagctcactatgaggctgctggattcgttggccaagcacccgccagaacagctgcatcaattgtagttgatatgattcgattgaagtgtatggccggacgagctgtattgattgctggaccacccgccactggaaaaactgcaatcgcgcttgcaatgtcacaggagttgggtgacggcgttccatttgtgccacttgttgcaagcgaggtcttttccaatgaagtaaagaaaacggaagtgctcatgagaagtttcagaagagctattggtctgcgtgttaaggaaaccaaagatgtgtacgagggagaagttacagagttgagccccgtcgaagcttcagataattccggaatgggaaaaaccatctctcatcttgtcctttctttgaaaacagcaaaaggaagcaagcaactgaaattggatccaagcatctatgactcgattttgaagcaacgagtagaagttggtgatgtcatctatattgaagcgaactcaggaatcgttaaacgagttggacggtgtgatgtatatgcatctgagtttgacctcgaggctgacgagtttgtgccaatgccgaaaggagatgttagaaagtctaaagatattgttcaaaacgtgtctttgcatgatctggatatcgcaaatgcccgtccacaaggacgtcaaggagatgttagtaacattgtttcacagctgatgactccaaagaaaactgaagttactgatcgtcttcgttctgaaatcaacaaagtagtcaacgaatacattgaaagtggagtggctgaactaatgcctggtgttcttttcatcgacgaggttcatatgcttgacgtagaatgttttacctatctctatcgcgcgctcgagtccccaatggcacccgtcgtggtttttgcaactaaccgtggaaccacaacagttcgtggactcggcgataaggcaccacatggaattcctccagaaatgctcgatcggctgatgattattccaacgatgaagtataacgaagaagacatccggaagattctcgttcatcgtaccgaagctgaaaatgttcaattcgaagaaaaagcatttgatcttctaactcgtctttgtgctcaaacgtgtggaagagaggtcattgaagtagaggacgtggaccgttgtaccaaattgttcatggatcgtggggagtcgttgaaaaaggccgaagaagagatgcgacaacctaaaaataagaagtga

SEQ ID NO: 20 - protein, 458 aa, Caenorhabditis elegans
MDMEVDEAISGTSSSRLAPIEEVKPTPKQIKRIAAHSHVKGLGIDTETQEAHYEAAGFVGQAPARTAASIVVDMIRLKCMAGRAVLIAGPPATGKTAIALAMSQELGDGVPFVPLVASEVFSNEVKKTEVLMRSFRRAIGLRVKETKDVYEGEVTELSPVEASDNSGMGKTISHLVLSLKTAKGSKQLKLDPSIYDSILKQRVEVGDVIYIEANSGIVKRVGRCDVYASEFDLEADEFVPMPKGDVRKSKDIVQNVSLHDLDIANARPQGRQGDVSNIVSQLMTPKKTEVTDRLRSEINKVVNEYIESGVAELMPGVLFIDEVHMLDVECFTYLYRALESPMAPVVVFATNRGTTTVRGLGDKAPHGIPPEMLDRLMIIPTMKYNEEDIRKILVHRTEAENVQFEEKAFDLLTRLCAQTCGREVIEVEDVDRCTKLFMDRGESLKKAEEEMRQPKNKK

SEQ ID NO: 21 - DNA, 1212 bp, Caenorhabditis elegans
  CDS (1)..(1212)
atggacgatttattgttgtcatcgaaattaaagcgagtagattcagcgacagggagtgtatcaccacgagtatgtgctgctgatgctttaccaacaactaccccagccgcttcaacagctttacgtcaaacacaaaaagctccaaaaactccatacgtgattctaccagcatcggatacttttgaaaaatttcgaaaaactagtagagtatctgttgatattggtggtacactgataaaagttgtatactcttctgtaatggatgaggagcttccagaagaatctctgaatggtcacactcggaaatatgcgtatgaagatggaaaacgagtactcgttaatttcaaaaaattcacggatatggatcggtttattaattttttaaaagaggtgtggactgatcgtaaaagaggagatgtgattcattgtacaggaggtggatcctataagtattcagagataataatgaaagaattgggagttcgtttacaacgaactgacgaaatgagatctctgatttttggtgttaatttcttactcagtacaaatgttgacgaaagtttcacttatcatcatgatgcgattggaagaaataaattccaatatcgtccgattgccgctgatcttatctacccattccttcttgtcaatattggaaccggtatcagtattctgaaagttgattcacctacaagttacgaacgagttggtggtagttcaatgggtggtggaacatttatgggacttggaagcctgctcacacctgctcaaaatttcgacgaacttcttgaaatggcaaatcgaggagatcatcgtaatgtagataagcttgtatgtgatatttatggcggagcatatgatgaacttggattgaaagctgacctgattgctggatcaatggctaaatgtaatcgattcgaagagacaactaaaaagcagcaacataaaccagaagatattgcaaaatctcttctgttgatggtcagcaacaatattggacagatggcatatttgtatggcactcggtataatttgaaaaggatatactttggaggatatttcatccgacaagatccaatcacaatgcggacgctttcttttgctataaattattggagcaagggagagattgaagcattgtatctgaaacacgaaggttatttgggtgccatgggctcgtttttggatgaggatggcaaattagacgattaa

SEQ ID NO: 22 - protein, 403 aa, Caenorhabditis elegans
MDDLLLSSKLKRVDSATGSVSPRVCAADALPTTTPAASTALRQTQKAPKTPYVILPASDTFEKFRKTSRVSVDIGGTLIKVVYSSVMDEELPEESLNGHTRKYAYEDGKRVLVNFKKFTDMDRFINFLKEVWTDRKRGDVIHCTGGGSYKYSEIIMKELGVRLQRTDEMRSLIFGVNFLLSTNVDESFTYHHDAIGRNKFQYRPIAADLIYPFLLVNIGTGISILKVDSPTSYERVGGSSMGGGTFMGLGSLLTPAQNFDELLEMANRGDHRNVDKLVCDIYGGAYDELGLKADLIAGSMAKCNRFEETTKKQQHKPEDIAKSLLLMVSNNIGQMAYLYGTRYNLKRIYFGGYFIRQDPITMRTLSFAINYWSKGEIEALYLKHEGYLGAMGSFLDEDGKLDD

SEQ ID NO: 23 - DNA, 1437 bp, Caenorhabditis elegans
  CDS (1)..(1437)
atgcacagagcacttctcaacgcctcgagacgggtggcaaccgtcagaagcatggcttctaccgttgaaggagacgctttccggctcagcgagtacagctcaaagtatttgggccatagaaaagccgctttcactgaaaaactagaaatcgtgaacgccgacgacacgccagcactgccaatctacagagtcaccaacgccgtcggtgacgtcatcgacaagtcgcaggacccaaatttcgacgagcaaacttcgctgaaaatgtacaaaacgatgacacagctgaatattatggatcggatcctgtatgattcgcagcgtcaaggacgcatctcgttttatatgacaagcttcggagaagaaggaaatcatgtcggaagtgccgccgctctcgagccacaggatctgatttatggacaatatcgtgaagccggggtcctgctgtggagaggatatactatggagaatttcatgaatcagtgctatggaaatgcggatgacttgggaaaaggtccgagaaaaaccttgattttgcttttaaaaaatcgatatttctggagaaagtcaacctatttttccgtttttttttcaatttttcttcacttcgattcccgaattttgcaaaaaaaaactgtgccgcacagaaaaagaggccgccaaatgccaatgcatttcggaacaaaagagcgaaactttgtcacaatctcatctccactgaccactcaacttccacaagccgtcggctcagcgtacgctttcaagcaacaaaaggataataatcgcatcgcagtcgtctatttcggagatggagccgcttctgaaggagatgctcacgcagcgttcaacttcgccgccactctcaaatgcccgattattttcttctgtagaaacaacggatacgccatttctacgccgactagtgaacagtatggaggagatggaattgctggaaagggaccggcttatggtcttcatactattagagtcgatggaaacgaccttctcgccgtctacaacgcgacaaaagaagcccgccgagtcgccctcaccaaccgcccagtgctcatcgaggccatgacctaccgcctcggccatcattcaacatccgacgactcgaccgcctaccgatcttccgatgaagttcaaacatggggagacaaggatcatccgatcactcgcttcaagaagtacatcacggaacgtggatggtggaatgaggagaaggagatggaatggcagaaagaggtgaagaagcgtgtgctcaccgagttcgccgcagctgagaagcggaagaaggcgcattatcatgatttgttcgaggatgtttatgatgagcttccactcagacttcgccgtcagagagatgagctggatgctcatgttgcggagtacaaggagcattatccgatgttggagactcttcaatcgaagccttaa

SEQ ID NO: 24 - protein, 478 aa, Caenorhabditis elegans
MHRALLNASRRVATVRSMASTVEGDAFRLSEYSSKYLGHRKAAFTEKLEIVNADDTPALPIYRVTNAVGDVIDKSQDPNFDEQTSLKMYKTMTQLNIMDRILYDSQRQGRISFYMTSFGEEGNHVGSAAALEPQDLIYGQYREAGVLLWRGYTMENFMNQCYGNADDLGKGPRKTLILLLKNRYFWRKSTYFSVFFSIFLHFDSRILQKKTVPHRKRGRQMPMHFGTKERNFVTISSPLTTQLPQAVGSAYAFKQQKDNNRIAVVYFGDGAASEGDAHAAFNFAATLKCPIIFFCRNNGYAISTPTSEQYGGDGIAGKGPAYGLHTIRVDGNDLLAVYNATKEARRVALTNRPVLIEAMTYRLGHHSTSDDSTAYRSSDEVQTWGDKDHPITRFKKYITERGWWNEEKEMEWQKEVKKRVLTEFAAAEKRKKAHYHDLFEDVYDELPLRLRRQRDELDAHVAEYKEHYPMLETLQSKP

SEQ ID NO: 25 - DNA, 2508 bp, Caenorhabditis elegans
  CDS (1)..(2508)
atgggctactcggagagtcgctcggagagcgttagctcgaaaggcaaaacatcatacggccatgaactcgaaacagtaccactaccggaaaagaaaatctacacaacatggcctgatatgatcaggcattggcctaaaacaacactgtgcatcgtgtccaacgaattctgcgaacgattctcatactatggaatgagaacggtcttgacattctacctgcttaacgtactaaagttcaccgactcacaatctacgatcttcttcaatggatttactgtgctttgctataccacaccacttctcggatcaattgttgcggacggatacattggaaaattctggacaatcttctctgtctcaattctatacgcaatcggacaagtcgtgctcgctctcgcttctactaagaattttcaatcttcagttcacccatggatggatctgtctggattactgatcattgcgttcggtaccggaggaatcaagccatgtgtgtctgcattcggaggagatcaattcgagttaggacaagaaagaatgctctcactcttcttttccatgttctacttttccatcaacgcaggatctatgatctctactttcatctctcccatcttcagatctcaaccatgccttggacaagattcctgctacccaatggcttttggtattccggctattcttatgattgttgcaacactggtatttatgggaggttcattttggtacaagaagaacccaccaaaggacaacgtgttcggagaagtatctcgtcttatgtttagagctgttggaaacaaaatgaagtcaggatccacaccaaaggaacactggctccttcactaccttactactcacgactgtgctcttgatgcaaaatgcctcgaactccaagctgagaaaagaaacaagaatctgtgccaaaagaaaaaattcatcgatgatgtccgttccctactccgtgttcttgtcatgttcttgccagtaccaatgttctgggcactttacgatcagcaaggatctgtctggctcattcaagctattcaaatggattgtcgtctttctgacacccttctccttcttccggatcaaatgcagacattgaacgccgtgcttattcttctctttatcccactctttcaggtcatcatctacccagtcgctgccaagtgtgtccgacttacacctttgagaaaaatggtcactggagggcttctcgcgtcacttgcctttttgatcactggatttgttcaacttcaagtcaatactactcttccaactctcccggaagaaggagaagcatcaattagtttctggaaccagtttgaaactgattgcaccattaccgttatgtctggaattcacaagagagttcttccccatgacaagtatcttcacgaagataaaaagaacaagtctggaatttataatctttttacaacaaaatcccccgcaaaaggtaatggcgattggactctcacatatgacctcagctatgacggcgcttgcggagatacatcaaaattggagaaaactgttaaagtgactgccaagagcaagaagatcatctacgttggagtcggatcatttggatattaccaaaacacagctaacactgataagccaactgatggaactggagaattctctatgggaattgtcactgtattcaattcttcatacggaggaaactttgccatgtgccgtcaaaatacaagcgactttgatgttaaccatccatgtaatccaagacatcctgctgacttttacttctgggaaaccgattataatagtcataccgatgacagagatcaaaacgctacaattactggatctcttagttcccaaccggctgtaacttacaagcagaaatctgtaaagccaggatattggcagttgtactaccttttgaatacaccaaaagatgtagaccggcagacttacaacaaaacagcaacactcgttgctccaacaaattacggttttcacagagttaagcagggaggtgtgttcatctatgcattgaccggaacatacgaaaatccaaagatccacgagcttcaaattgtacagtccaatagtgtgtcgattctttggcagattccacaaatcgttgtcatcacggcagcagaaattttgttctcaattaccggatacgaatttgcatattctcagtctgccccatcaatgaaagctctggtacaagccctctggctcttgaccactgctgctggagattccatcattgtagtcatcacaatccttaacttatttgaaaatatggccgtcgaattctttgtctatgctgctgcgatgtttgtagttattgccatctttgctcttctctccattttctattatacttacaattattatacaactgatgaagaggacggtgaaattggagttgatgatgaggaagaaattgaggatcacaatccacgatattcaattgataacaaaggtttccatccggacgaaaaagatactttcgatatgcatttttaa

SEQ ID NO: 26 - protein, 835 aa, Caenorhabditis elegans
MGYSESRSESVSSKGKTSYGHELETVPLPEKKIYTTWPDMIRHWPKTTLCIVSNEFCERFSYYGMRTVLTFYLLNVLKFTDSQSTIFFNGFTVLCYTTPLLGSIVADGYIGKFWTIFSVSILYAIGQVVLALASTKNFQSSVHPWMDLSGLLIIAFGTGGIKPCVSAFGGDQFELGQERMLSLFFSMFYFSINAGSMISTFISPIFRSQPCLGQDSCYPMAFGIPAILMIVATLVFMGGSFWYKKNPPKDNVFGEVSRLMFRAVGNKMKSGSTPKEHWLLHYLTTHDCALDAKCLELQAEKRNKNLCQKKKFIDDVRSLLRVLVMFLPVPMFWALYDQQGSVWLIQAIQMDCRLSDTLLLLPDQMQTLNAVLILLFIPLFQVIIYPVAAKCVRLTPLRKMVTGGLLASLAFLITGFVQLQVNTTLPTLPEEGEASISFWNQFETDCTITVMSGIHKRVLPHDKYLHEDKKNKSGIYNLFTTKSPAKGNGDWTLTYDLSYDGACGDTSKLEKTVKVTAKSKKIIYVGVGSFGYYQNTANTDKPTDGTGEFSMGIVTVFNSSYGGNFAMCRQNTSDFDVNHPCNPRHPADFYFWETDYNSHTDDRDQNATITGSLSSQPAVTYKQKSVKPGYWQLYYLLNTPKDVDRQTYNKTATLVAPTNYGFHRVKQGGVFIYALTGTYENPKIHELQIVQSNSVSILWQIPQIVVITAAEILFSITGYEFAYSQSAPSMKALVQALWLLTTAAGDSIIVVITILNLFENMAVEFFVYAAAMFVVIAIFALLSIFYYTYNYYTTDEEDGEIGVDDEEEIEDHNPRYSIDNKGFHPDEKDTFDMHF

SEQ ID NO: 27 - DNA, 1899 bp, Caenorhabditis elegans
  CDS (1)..(1899)
atggaggatctcacaccaactaacacgtcgctcgacaccacaactactaacaatgacacgacatcggatcgtgaagcggcgccaacgacgctcaacttaacaccaacagcaagtgaatcggagaacagcttatccccagtcaccgccgaagatctcatagctaaaagcattaaagaaggatgtccgaagagaacttccaacgacttcatgtttcttcagagtatgggcgaaggagcctacagccaggtattccgatgtcgcgaagtggcaacagatgcgatgttcgccgtcaaagtgctccagaagtcgtacctcaaccgccatcaaaaaatggacgcaatcattcgcgagaagaatatcttaacatacctgtcacaagaatgcggtggtcatccgtttgtcacacagctctacacacattttcacgaccaggctagaatttatttcgtgatcggacttgttgaaaatggtgatcttggcgagtcgctgtgccattttggatcattcgacatgctcacctcaaaattctttgcctcggaaatcctcaccggactgcaattcctacacgacaacaaaattgtgcacagagacatgaagccggacaatgtgctcatccagaaagacggtcacattctcatcacagattttggaagtgcccaggcgtttggcggtctccaactgtcacaggagggctttacggatgcgaatcaggcaagctcgcgatcttcggattctggatcgccgccgccaactcgattctattcggatgaggaggaagagaacactgctcgacgtaccacatttgttggaactgctctctacgtgagcccggagatgctagctgacggagatgtgggaccacaaaccgacatttggggattgggatgtatccttttccagtgtctagccggacagccaccattcagagccgtcaaccagtaccatcttttgaaaagaatccaggagttggatttctcgttcccagaaggatttccagaggaagcgtcggaaattatcgcaaagattttggtgcgcgacccgagtacccgtatcaccagtcaagaacttatggctcacaagttttttgaaaacgttgactgggtgaacattgcaaatatcaagccaccagtcctgcacgcctacattccagccacatttggcgagccggagtactactctaacattgggcctgtcgagccgggacttgatgatcgtgccttgttccgtttgatgaatttgggaaatgatgctagcgcatcacagccatcaacaccgtctaacgtggaacatcgcggagacccatttgtttcggaaattgcaccacgcgccaattcggaagccgaaaagaaccgcgccgcacgtgcgcagaagctcgaagagcaacgtgtcaaaaacccattccacatcttcaccaacaactcgctcattttgaaacaaggatatttggaaaagaagcgaggattgtttgccagacgccgaatgttcctgttgaccgaaggaccgcatctcttgtacattgatgtgccgaatcttgtgctcaaaggagaggtaccatggacgccgtgcatgcaggtggagctaaaaaactcgggaactttctttatacatacgcccaaccgcgtctactacttgtttgatctcgaaaagaaagcagatgagtggtgtaaggctatcaatgatgttcgcaagcggtactcggtgactatcgaaaagacttttaactctgcgatgcgtgacggaacatttggcagcatttatggaaagaaaaagtccagaaaggaaatgatgcgtgaacagaaggcgctgcgccgcaaacaagaaaaggaggagaaaaaggcgctaaaagccgagcaagtgagcaagaagctttcaatgcaaatggacaagaagtcgccttga

SEQ ID NO: 28 - protein, 632 aa, Caenorhabditis elegans
MEDLTPTNTSLDTTTTNNDTTSDREAAPTTLNLTPTASESENSLSPVTAEDLIAKSIKEGCPKRTSNDFMFLQSMGEGAYSQVFRCREVATDAMFAVKVLQKSYLNRHQKMDAIIREKNILTYLSQECGGHPFVTQLYTHFHDQARIYFVIGLVENGDLGESLCHFGSFDMLTSKFFASEILTGLQFLHDNKIVHRDMKPDNVLIQKDGHILITDFGSAQAFGGLQLSQEGFTDANQASSRSSDSGSPPPTRFYSDEEEENTARRTTFVGTALYVSPEMLADGDVGPQTDIWGLGCILFQCLAGQPPFRAVNQYHLLKRIQELDFSFPEGFPEEASEIIAKILVRDPSTRITSQELMAHKFFENVDWVNIANIKPPVLHAYIPATFGEPEYYSNIGPVEPGLDDRALFRLMNLGNDASASQPSTPSNVEHRGDPFVSEIAPRANSEAEKNRAARAQKLEEQRVKNPFHIFTNNSLILKQGYLEKKRGLFARRRMFLLTEGPHLLYIDVPNLVLKGEVPWTPCMQVELKNSGTFFIHTPNRVYYLFDLEKKADEWCKAINDVRKRYSVTIEKTFNSAMRDGTFGSIYGKKKSRKEMMREQKALRRKQEKEEKKALKAEQVSKKLSMQMDKKSP

SEQ ID NO: 29 - DNA, 5532 bp, Caenorhabditis elegans
  CDS (1)..(5532)
atgaatattgtcagatgtcggagacgacacaaaattttggaaaatttggaagaagagaatctcggcccgagctgctcgtcgacgacttcaacaaccgctgccaccgaagctctcggaacaaccactgaggatatgaggcttaagcagcagcgaagctcgtcgcgtgccacggagcacgatattgtcgacggcaatcaccacgacgacgagcacatcacaatgagacggcttcgacttgtcaaaaattcgcggacgcggcgtagaacgacgcccgattcaagtatggactgctatgaggaaaacccgccatcacaaaaaacttcaataaattattcttggatttctaaaaagtcatcaatgacgtcattaatgcttttactgctattcgcttttgtacagccgtgtgcctcaatagtcgaaaaacgatgcggcccaatcgatattcgaaataggccgtgggatattaagccgcaatggtcgaaacttggtgatccgaacgaaaaagatttggctggtcagagaatggtcaactgcacagtggtggaaggttcgctgacaatctcatttgtactgaaacacaagacaaaagcacaagaagaaatgcatcgaagtctacagccaagatattcccaagacgaatttatcacttttccgcatctacgtgaaattactggaactctgctcgtttttgagactgaaggattagtggatttgcgtaaaattttcccaaatcttcgtgtaattggaggccgttcgctgattcaacactatgcgctgataatttatcgaaatccggatttggagatcggtcttgacaagctttccgtaattcgaaatggtggtgtacggataatcgataatcgaaaactgtgctacacgaaaacgattgattggaaacatttgatcacttcttccatcaacgatgttgtcgttgataatgctgccgagtacgctgtcactgagactggattgatgtgcccacgtggagcttgcgaagaggataaaggcgaatcaaagtgtcattatttggaggaaaagaatcaggaacaaggtgtcgaaagagttcagagttgttggtcgaacaccacttgccaaaagtcttgtgcttatgatcgtcttcttccaacgaaagaaatcggaccgggatgtgatgcgaacggcgatcgatgtcacgatcaatgcgtgggcggttgtgagcgtgtgaatgatgcgacagcatgccacgcgtgcaagaatgtctatcacaagggaaagtgtatcgaaaagtgtgatgctcacctgtaccttctccttcaacgtcgttgtgtgacccgtgagcagtgtctgcagctgaatccggtgctctcgaacaaaacagtgcctatcaaggcgacggcaggcctttgctcggataaatgtcccgatggttatcaaatcaacccggatgatcatcgagaatgccgaaaatgcgttggcaagtgtgagattgtgtgcgagatcaatcacgtcattgatacgtttccgaaggcacaggcgatcaggctatgcaatattattgacggaaatctgacgatcgagattcgcggaaaacaggattcgggaatggcgtccgagttgaaggatatatttgcgaacattcacacgatcaccggctacctgttggtacgtcaatcgtcaccgtttatctcgttgaacatgttccggaatttacgacgtattgaggcaaagtcactgttcagaaatctatatgctatcacagtttttgaaaatccgaatttaaaaaagctattcgattcaacgacggatttgacgcttgatcgtggaactgtgtcaattgccaataacaagatgttatgcttcaagtatatcaagcagctaatgtcaaagttaaatataccactcgatccgatagatcaatcagaagggacaaatggtgagaaggcaatctgtgaggatatggcaatcaacgtgagcatcacagcggtcaacgcggactcggtcttctttagttggccctcattcaacattaccgatatagatcagcgaaagtttctcggctacgagctcttcttcaaagaagtcccacgaatcgatgagaacatgacgatcgaagaggatcgaagtgcgtgtgtcgattcgtggcagagtgtcttcaaacagtactacgagacgtcgaacggtgaaccgaccccggacatttttatggatattggaccgcgcgagcgaattcggccgaatacgctctacgcgtactatgtggcgacgcagatggtgttgcatgccggtgcgaagaacggtgtatcgaagattggttttgtgaggacgagctactatacgcctgatcctccgacgttggcactagcgcaagtcgattcggacgctattcatattacgtgggaagcgccgctccaaccgaacggagacctcacgcattacacaattatgtggcgtgagaatgaagtgagcccgtacgaggaagccgaaaagttttgtacagatgcaagcacccccgcaaatcgacaacacacgaaagatccgaaagagacgattgtagccgataagccagtcgatattccgtcatcacgtaccgtagctccgacacttttgactatgatgggtcacgaagatcagcagaaaacgtgcgctgcaacgcccggttgttgttcgtgttcggctatcgaagaatcatcggaacagaacaagaagaagcgaccggatccgatgtcggcgatcgaatcatctgcatttgagaataagctgttggatgaggttttaatgccgagagacacgatgcgagtgagacgatcaattgaagacgcgaatcgagtcagtgaagagttggaaaaagctgaaaatttgggaaaagctccaaaaactctcggtggaaagaagccgctgatccatatttcgaagaagaagccgtcgagcagcagcaccacatccacaccggctccaacgatcgcatcaatgtatgccttaacaaggaaaccgactacggtgccgggaacaaggattcggctctacgagatctacgaacctttacccggaagctgggcgattaatgtatcagctctggcattggataatagttatgtgatacgaaatttgaagcattacacactttatgcgatttctctatccgcgtgccaaaacatgacagtacccggagcatcttgctcaatatcccatcgtgcgggagcattgaaacgaacaaaacacatcacagacattgataaagtgttgaatgaaacaattgaatggagatttatgaataatagtcaacaagtcaacgtgacgtgggatccaccgactgaagtgaatggtggaatattcggttatgttgtaaagcttaagtcaaaagtcgatggatcaattgttatgacgagatgtgtcggtgcgaagagaggatattcaacacggaatcagggtgtcctattccagaatttggccgatggacgttattttgtctcagtaacggcgacctctgtacacggcgctggaccggaagccgaatcctccgacccaatcgtcgtcatgacgccaggcttcttcactgtggaaatcattctcggcatgcttctcgtctttttgattttaatgtcaattgccggttgtataatctactactacattcaagtacgctacggcaaaaaagtgaaagctctatctgactttatgcaattgaatcccgaatattgtgtggacaataagtacaatgcagacgattgggagctacggcaggatgatgttgtgctcggacaacagtgtggagagggatcattcggaaaagtgtacctaggaactggaaataatgttgtttctctgatgggtgatcgtttcggaccgtgtgctattaagattaatgtagatgatccagcgtcgactgagaatctcaactatctcatggaagctaatattatgaagaactttaagactaactttatcgtcaaactgtacggagttatctctactgtacaaccagcgatggttgtgatggaaatgatggatcttggaaatctccgtgactatctccgatcgaaacgcgaagacgaagtgttcaatgagacggactgcaactttttcgacataatcccgagggataaattccatgagtgggccgcacagatttgtgatggtatggcgtacctggagtcgctcaagttttgccatcgagatctcgccgcacgtaattgcatgataaatcgggatgagactgtcaagattggagatttcggaatggctcgtgatctattctatcatgactattataagccatcgggcaagcgtatgatgcctgttcgatggatgtcacccgagtcgttgaaagacggaaagtttgactcgaaatctgatgtttggagcttcggagttgttctctatgaaatggttacactcggtgctcagccatatattggtttgagtaatgatgaggtgttgaattatattggaatggcccggaaggttatcaagaagcccgaatgttgtgaaaactattggtataaggtgatgaaaatgtgctggagatactcacctcgggatcgtccgacgttcctccagctcgttcatcttctagcagctgaagcttcaccagaattccgagatttatcatttgtcctaaccgataatcaaatgatccttgacgattcagaagcactggatcttgatgatattgatgatactgatatgaatgatcaggttgtcgaggtggcaccggatgttgagaacgtcgaggttcagagtgattcggaacgtcggaatacggattcaataccgttgaaacagtttaagacgatccctccgatcaatgcgacgacgagtcattcgacaatatcgattgatgagacaccgatgaaagcgaagcagcgagaaggatcgctggatgaggagtacgcattgatgaatcatagtggaggtccgagtgatgcggaagttcggacgtatgctggtgatggagattatgtggagagagatgttcgagagaatgatgtgccaacgcgacgaaatactggtgcatcaacatcaagttacacaggtggtggtccatattgcctaacaaatcgtggtggttcaaatgaacgaggagccggtttcggtgaagcagtacgattaactgatggtgttggaagtggacatttaaatgatgatgattatgttgaaaaagagatatcatccatggatacgcgccggagcacgggcgcctcgagctcttcctacggtgttccacagacgaattggagtggaaatcgtggtgccacgtattatacgagtaaagctcaacaggcagcaactgcagcagcagcagcagcagcagctctccaacagcaacaaaatggtggtcgaggcgatcgattaactcaactacccggaactggacatttacaatcgacacgtggtggacaagatggagattatattgaaactgaaccgaaaaattatagaaataatggatctccatcgcgaaacggcaacagccgtgacattttcaacggacgttcggctttcggtgaaaatgagcatctaatcgaggataatgagcatcatccacttgtctga

SEQ ID NO: 30 - protein, 1843 aa, Caenorhabditis elegans
MNIVRCRRRHKILENLEEENLGPSCSSTTSTTAATEALGTTTEDMRLKQQRSSSRATEHDIVDGNHHDDEHITMRRLRLVKNSRTRRRTTPDSSMDCYEENPPSQKTSINYSWISKKSSMTSLMLLLLFAFVQPCASIVEKRCGPIDIRNRPWDIKPQWSKLGDPNEKDLAGQRMVNCTVVEGSLTISFVLKHKTKAQEEMHRSLQPRYSQDEFITFPHLREITGTLLVFETEGLVDLRKIFPNLRVIGGRSLIQHYALIIYRNPDLEIGLDKLSVIRNGGVRIIDNRKLCYTKTIDWKHLITSSINDVVVDNAAEYAVTETGLMCPRGACEEDKGESKCHYLEEKNQEQGVERVQSCWSNTTCQKSCAYDRLLPTKEIGPGCDANGDRCHDQCVGGCERVNDATACHACKNVYHKGKCIEKCDAHLYLLLQRRCVTREQCLQLNPVLSNKTVPIKATAGLCSDKCPDGYQINPDDHRECRKCVGKCEIVCEINHVIDTFPKAQAIRLCNIIDGNLTIEIRGKQDSGMASELKDIFANIHTITGYLLVRQSSPFISLNMFRNLRRIEAKSLFRNLYAITVFENPNLKKLFDSTTDLTLDRGTVSIANNKMLCFKYIKQLMSKLNIPLDPIDQSEGTNGEKAICEDMAINVSITAVNADSVFFSWPSFNITDIDQRKFLGYELFFKEVPRIDENMTIEEDRSACVDSWQSVFKQYYETSNGEPTPDIFMDIGPRERIRPNTLYAYYVATQMVLHAGAKNGVSKIGFVRTSYYTPDPPTLALAQVDSDAIHITWEAPLQPNGDLTHYTIMWRENEVSPYEEAEKFCTDASTPANRQHTKDPKETIVADKPVDIPSSRTVAPTLLTMMGHEDQQKTCAATPGCCSCSAIEESSEQNKKKRPDPMSAIESSAFENKLLDEVLMPRDTMRVRRSIEDANRVSEELEKAENLGKAPKTLGGKKPLIHISKKKPSSSSTTSTPAPTIASMYALTRKPTTVPGTRIRLYEIYEPLPGSWAINVSALALDNSYVIRNLKHYTLYAISLSACQNMTVPGASCSISHRAGALKRTKHITDIDKVLNETIEWRFMNNSQQVNVTWDPPTEVNGGIFGYVVKLKSKVDGSIVMTRCVGAKRGYSTRNQGVLFQNLADGRYFVSVTATSVHGAGPEAESSDPIVVMTPGFFTVEIILGMLLVFLILMSIAGCIIYYYIQVRYGKKVKALSDFMQLNPEYCVDNKYNADDWELRQDDVVLGQQCGEGSFGKVYLGTGNNVVSLMGDRFGPCAIKINVDDPASTENLNYLMEANIMKNFKTNFIVKLYGVISTVQPAMVVMEMMDLGNLRDYLRSKREDEVFNETDCNFFDIIPRDKFHEWAAQICDGMAYLESLKFCHRDLAARNCMINRDETVKIGDFGMARDLFYHDYYKPSGKRMMPVRWMSPESLKDGKFDSKSDVWSFGVVLYEMVTLGAQPYIGLSNDEVLNYIGMARKVIKKPECCENYWYKVMKMCWRYSPRDRPTFLQLVHLLAAEASPEFRDLSFVLTDNQMILDDSEALDLDDIDDTDMNDQVVEVAPDVENVEVQSDSERRNTDSIPLKQFKTIPPINATTSHSTISIDETPMKAKQREGSLDEEYALMNHSGGPSDAEVRTYAGDGDYVERDVRENDVPTRRNTGASTSSYTGGGPYCLTNRGGSNERGAGFGEAVRLTDGVGSGHLNDDDYVEKEISSMDTRRSTGASSSSYGVPQTNWSGNRGATYYTSKAQQAATAAAAAAAALQQQQNGGRGDRLTQLPGTGHLQSTRGGQDGDYIETEPKNYRNNGSPSRNGNSRDIFNGRSAFGENEHLIEDNEHHPLV

SEQ ID NO: 31 - DNA, 2319 bp, Caenorhabditis elegans
  CDS (1)..(2319)
atgtcgtcgcgtaaacgaggaataactccatcgcgagaccaagtccgccggaagaagttatcgattgaagaaaccgacagtatcgaagtcgtttgtcgtctttgtccatatactggctcgactccaagtcttattgcaattgatgagggatctattcaaactgttcttccaccagcacagttcagacgcgaaaacgctccacaagttgagaaagtgtttagatttggacgagttttttcggaaaatgatggacaagctactgtttttgagcggacatctgttgatttaattttaaacctattgaaaggtcagaattcgttgttattcacttatggagttactggatctggaaaaacgtatacaatgacgggaaaacccactgaaaccggcacaggactactgccgcgtacattggatgtaattttcaatagtattaataatcgagttgagaaatgcatcttctatccatcagcactgaatacattcgagatccgtgccacattggatgctcacttgaaacgccatcaaatggctgcagaccgtctttccacatcacgcgaaatcactgatcgttactgtgaagctataaagctgtcaggctacaacgacgatatggtttgctcggttttcgtaacctacgtcgaaatctacaacaattattgctacgatttgttggaagacgccagaaatggagtattgacgaagcgtgaaattcgtcatgatcgtcagcaacagatgtacgtcgacggagccaaagatgttgaagtctcgtcaagcgaggaagctctcgaagtgttctgccttggagaagaacgtcgtcgtgtatcgtccactcttctcaacaaagattcatcccgttctcattccgtattcactatcaaattggttatggctccgagagcctacgagacgaaaagcgtgtatccaacaatggactcatcgcaaattatcgtttcgcagttatgtttggtagatttggctggatctgagagagcaaagcgcacacagaatgttggtgaacgtcttgcggaagccaactcgatcaatcagtccctcatgactcttcgtcagtgtattgaagtactccgtcgtaaccaaaagagttcctcacaaaaccttgagcaagttccatatcgccagtcaaaattgactcatttattcaaaaactatctggaaggaaatggaaaaatcagaatggttatttgtgtgaatccaaagcctgatgattacgatgaaaacatgagtgctctagctttcgctgaagaatcacaaacaattgaagtgaaaaagcaagttgaacgaatgccatccgagcggattcctcattcattcttcacccaatggaattctgagctagatggatctgttcgtatggaggatgatggaagtagagaaattccgtgcccaccgacattctgtttgacagattgcaatgacaaagatacggtagattccatgtataagtatgctcggaaactttcatctcttcaaaattcatcagaagagggaccatcttcaactcttcttactatgattcgccaatacatgatggaagcagactaccagcgagtagagattgcacgtctcaaagattctctaaacgacaaggatgaagaaatcaagaagcttcgaggtttctgctcaagatataagcgtgagaacgcttcgatgaaggaacgaattgcctcgtgtgagcaaggagagcaagagaatgctctggttatggaaaagcttatggaacaaaaaatggaggacaggaagattattcagtcacagaagaaggcgatgagaaatgttcgtggaattattgataatccatccccgtctgttgcttctcttcgatcacgatttgatcaagagaatgtggctcatccaacagctccaatccaaactccaccaccgccatatcaaactccaggacgtgctccagtcttcaaaaaacggttagaagccactacatcgaccactgtaatgtctggatcttcaagtggaggaagtggtcaacagggttacgttaatccaaaatatcaaagaagatccaagtctgcatctcgtctattggatcatcagccacttcatcgagttccaacaggaacggttcttcagtctcgtacaccggctaacgccatacggactactaaacctgagatgcatcagttgaacaaatccggagaataccgtctcacgcatcaagaagttgacgatgaaggaaacattagcacgaatatagtgaaggtaaattccttggtttccacccaaaaacacgcctgtaccgtcccgttgtcgttttcccgtgtccttatcacccatctttcctga

SEQ ID NO: 32 - protein, 772 aa, Caenorhabditis elegans
MSSRKRGITPSRDQVRRKKLSIEETDSIEVVCRLCPYTGSTPSLIAIDEGSIQTVLPPAQFRRENAPQVEKVFRFGRVFSENDGQATVFERTSVDLILNLLKGQNSLLFTYGVTGSGKTYTMTGKPTETGTGLLPRTLDVIFNSINNRVEKCIFYPSALNTFEIRATLDAHLKRHQMAADRLSTSREITDRYCEAIKLSGYNDDMVCSVFVTYVEIYNNYCYDLLEDARNGVLTKREIRHDRQQQMYVDGAKDVEVSSSEEALEVFCLGEERRRVSSTLLNKDSSRSHSVFTIKLVMAPRAYETKSVYPTMDSSQIIVSQLCLVDLAGSERAKRTQNVGERLAEANSINQSLMTLRQCIEVLRRNQKSSSQNLEQVPYRQSKLTHLFKNYLEGNGKIRMVICVNPKPDDYDENMSALAFAEESQTIEVKKQVERMPSERIPHSFFTQWNSELDGSVRMEDDGSREIPCPPTFCLTDCNDKDTVDSMYKYARKLSSLQNSSEEGPSSTLLTMIRQYMMEADYQRVEIARLKDSLNDKDEEIKKLRGFCSRYKRENASMKERIASCEQGEQENALVMEKLMEQKMEDRKIIQSQKKAMRNVRGIIDNPSPSVASLRSRFDQENVAHPTAPIQTPPPPYQTPGRAPVFKKRLEATTSTTVMSGSSSGGSGQQGYVNPKYQRRSKSASRLLDHQPLHRVPTGTVLQSRTPANAIRTTKPEMHQLNKSGEYRLTHQEVDDEGNISTNIVKVNSLVSTQKHACTVPLSFSRVLITHLS

SEQ ID NO: 33 - DNA, 3174 bp, Caenorhabditis elegans
  CDS (1)..(3174)
atggcgtggagatttgcagcgtcgaaattcaagaacacgacgccaaaggttccgaagaaggaggagacaatcttcgatgttcccgtcggcaatctctcctgcacgaatgacggaatccacgccagcgccgacttcctcgctttccacattgagggagaaggtggcaaactcggagttctgcccatcactgcgaagggacgacgcacccgcaacgatatcggaattatcgcggctcacggagagcaagtagcggatttcggattcttgacgttcgccgatgagctgctcgccacgtgcagccgagatgaacccgtaaaaatctggaagctctcccgggatcactctccaaaactggccacagaaatcgacgttggaggtggcaacgtgattgcggaatgtcttcgagctcattccacggccgataacattttggcagtcggctcccacggttcgacgtacatcacggacatctccacgggaaagacggctgtcgagctctccggagtgacggataaagttcaatcgatggactggagtgaggatggtaaacttctggcggtcagtggcgacaagggacgtcagattgttgtgtacgacccgcgtgctagcatggagccaatacaaacgctcgagggacatggtggaatgggcagagaggcccgtgtgctctttgctggaaaccgactcatcagcactggtttcactacgaaacgaatccaagaagtgcgcgcgtacgatactggaaaatggggagcacccgtgcatacacaggagttcgtctccaccaccggtgtactcatcccgcattacgacgccgacactcgtctcgtcttcttgtctggcaagggaaccaataagttatttatgctggagatgcaggatcgtcaaccctatctttcgcatgtcttcgagcttacactgccagagcagacactcggtgcgacgattggcgccaagcggcgagtacatgttatggatggagaggttgatacctactaccagcttacgaaaagttcgattgtgccaactccatgcatcgtgccacgaagatcctatcgtgatttccacagcgatctgttcccagagacacgtggtgccgagccaggatgcaccgccggcgagtggttgaatgggacaaatgcagttccgcagaaagttagcatggctccgtcgcaaagctcctcatcgccaccgcctccagagccagttccaactccgaaggttgctcaaacaccagctccagttccagtaccaacaccagcagccgcacctcgtcccatgtccaacaataattcatcgtcgaacaacgtgccgagcgtccaggaacaacattcggttccaaagaaagaagaggttcgagaactcgattacaggccttacgaaaaggagaatggagttcacaccccaaatgccgagacaaatagcactcagggaaactcgtcaccaatctccaccatctctccggagccagtcacgattgtgaagcccgcaagcacgcctgcaaccgactcagtgtcaactccaagcgtcgttggaccggcatttggtaaaaaggttccggagcagccaccagtgaacttccgtaagccgatcggagcctcgaatcgtgtgccactctcgcaaagagttcgtccgaagtcgtgtgttgtcggtcagatcacgtcgaagttccgtcacgtggatggtcagcaaggaacgaaatctggcgccgtgttctcgaatcttcgcaatgtgaacacgcgtctgccgccagagtccaacggtgtctgctgctcgaacaaatttgcggcggttcctctcgccggtctaggagtcattgggatctatgatgtgaatgagcctggcaagttgcccgatggagttatggacggaatcttcaacaagacgcttgtcaccgatttgcactggaatccgttcgacgatgaacagctcgccgtaggaaccgactgtggacagatcaatctgtggcgtctaaccacgaacgatggtccacggaatgagatggaacccgagaagattatcaagattggaggtgagaagatcacttcgttgcgttggcatccacttgcgtcggatctcttggccgtggcgctttcgaatagtacaatcgagctgtgggatgtggcaaatgcgaagctttacagccggttcgtcaaccataccggagggatcttgggaatcgcatggtcggctgatggtcggcggatcgcttcagtcggaaaggacgcgacgctctttgtgcatgagccggcgagccgcgagcaacgggtctacgaacggaaaacagttgtcgagtcgactcgtgccgcccgtgtgctcttcgcctgtgacgatcggattgtgattgtggtcgggatgacgaagagctcgcagcgacaggttcagatgtatgatgcgcagacagtggatcttcgacacatttacactcaagtgatcgattcggcgacacagcccctggtgcctcactatgattacgattcgaatgtgcttttccttagcggaaaaggtgatcgatttgtgaacatgttcgaggtgatctatgattcgccgtatctgcttccgttggcaccatttatgtcgcctgttggaagtcaaggaatcgcgttccatcagaaactgaaatgtaacgtgatggctgtcgaatttcaagtttgctggcgcctctcggacaaaaatctggagaagattacgttccgtgttccacgtatcaagaaggacgtcttccaagatgacttgttcccggattcacttgtcacatgggagcccgtcacgactggaacaaaatggatgctcggagagcaagctgcacccgtgttcagatcactcaagccggatggcgtgttctcgtcgattcctcgcgcgatcactgcatctgttcgtcactcggaaatgccatcatcgtcgtccacgacaaattctgccgcacagacaccatctacttcagtcacacattcgactacggagaagcatcatcatcaccagcaccaccagcatcaggagccaacatcagtgccgacaccatcgtcgcgaaacatgcaaagctgtggagtcgaaagcactcaacagccggaccgtaaacaggtggccgctgcgtggtcgacaaaaatcgacgtggacacgcgcctcgaacaagatcaaatggagggtgtcgatgaggcggaatgggacaaatag

SEQ ID NO: 34 - protein, 1057 aa, Caenorhabditis elegans
MAWRFAASKFKNTTPKVPKKEETIFDVPVGNLSCTNDGIHASADFLAFHIEGEGGKLGVLPITAKGRRTRNDIGIIAAHGEQVADFGFLTFADELLATCSRDEPVKIWKLSRDHSPKLATEIDVGGGNVIAECLRAHSTADNILAVGSHGSTYITDISTGKTAVELSGVTDKVQSMDWSEDGKLLAVSGDKGRQIVVYDPRASMEPIQTLEGHGGMGREARVLFAGNRLISTGFTTKRIQEVRAYDTGKWGAPVHTQEFVSTTGVLIPHYDADTRLVFLSGKGTNKLFMLEMQDRQPYLSHVFELTLPEQTLGATIGAKRRVHVMDGEVDTYYQLTKSSIVPTPCIVPRRSYRDFHSDLFPETRGAEPGCTAGEWLNGTNAVPQKVSMAPSQSSSSPPPPEPVPTPKVAQTPAPVPVPTPAAAPRPMSNNNSSSNNVPSVQEQHSVPKKEEVRELDYRPYEKENGVHTPNAETNSTQGNSSPISTISPEPVTIVKPASTPATDSVSTPSVVGPAFGKKVPEQPPVNFRKPIGASNRVPLSQRVRPKSCVVGQITSKFRHVDGQQGTKSGAVFSNLRNVNTRLPPESNGVCCSNKFAAVPLAGLGVIGIYDVNEPGKLPDGVMDGIFNKTLVTDLHWNPFDDEQLAVGTDCGQINLWRLTTNDGPRNEMEPEKIIKIGGEKITSLRWHPLASDLLAVALSNSTIELWDVANAKLYSRFVNHTGGILGIAWSADGRRIASVGKDATLFVHEPASREQRVYERKTVVESTRAARVLFACDDRIVIVVGMTKSSQRQVQMYDAQTVDLRHIYTQVIDSATQPLVPHYDYDSNVLFLSGKGDRFVNMFEVIYDSPYLLPLAPFMSPVGSQGIAFHQKLKCNVMAVEFQVCWRLSDKNLEKITFRVPRIKKDVFQDDLFPDSLVTWEPVTTGTKWMLGEQAAPVFRSLKPDGVFSSIPRAITASVRHSEMPSSSSTTNSAAQTPSTSVTHSTTEKHHHHQHHQHQEPTSVPTPSSRNMQSCGVESTQQPDRKQVAAAWSTKIDVDTRLEQDQMEGVDEAEWDK

SEQ ID NO: 35 - DNA, 657 bp, Caenorhabditis elegans
  CDS (1)..(657)
atgctgcaatctactgctcgcactgcttcaaagcttgttcaaccggttgcgggagttctcgccgtccgctccaagcacactctcccagatctcccattcgactatgcagatttggaacctgtaatcagccatgaaatcatgcagcttcatcatcaaaagcatcatgccacctacgtgaacaatctcaatcagatcgaggagaaacttcacgaggctgtttcgaaagggaatctaaaagaagcaattgctctccaaccagcgctgaaattcaatggtggtggacacatcaatcattctatcttctggaccaacttggctaaggatggtggagaaccttcaaaggagctgatggacactattaagcgcgacttcggttccctggataacttgcaaaaacgtctttctgacatcactattgcggttcaaggctctggctggggatggttgggatattgcaagaaagacaaaatcttgaagatcgccacctgtgcaaaccaggatcctttggaaggaatggtcccactttttggaattgacgtttgggagcacgcctactacttgcagtacaaaaatgtccgcccagactatgtccatgctatttggaagattgccaactggaagaatatcagcgagagatttgccaatgctcgacaataa

SEQ ID NO: 36 - protein, 218 aa, Caenorhabditis elegans
MLQSTARTASKLVQPVAGVLAVRSKHTLPDLPFDYADLEPVISHEIMQLHHQKHHATYVNNLNQIEEKLHEAVSKGNLKEAIALQPALKFNGGGHINHSIFWTNLAKDGGEPSKELMDTIKRDFGSLDNLQKRLSDITIAVQGSGWGWLGYCKKDKILKIATCANQDPLEGMVPLFGIDVWEHAYYLQYKNVRPDYVHAIWKIANWKNISERFANARQ

SEQ ID NO: 37 - DNA, 1194 bp, Caenorhabditis elegans
  CDS (1)..(1194)
atgtctttattcgcgagacaacttcaatccttgactgccagtggaatccgcacacagcaagttcgtcttgcgagcacggaagtatcattccacacaaagccatgcaaacttcataaacttgacaatggaccaaacacctctgtcaccttgaacagagaagatgctctcaagtactaccgtgacatgcaggtcattcgtcgcatggagtctgctgctggaaacttgtacaaggagaagaaaatccgcggtttctgtcatctctactctggtcaagaagcttgtgccgtcggaatgaaggctgcaatgacagaaggagatgccgtcatcactgcttaccgttgtcacggatggacctggcttcttggagctaccgtaaccgaggttcttgctgaattgaccggtagagttgccggaaatgttcacggaaaaggaggatccatgcatatgtacacaaagaatttctatggaggaaacggaattgttggagcacaacagccacttggagctggagttgcattggccatgaaataccgtgaacaaaagaatgtatgtgttactttgtatggtgatggtgccgctaatcaaggacaacttttcgaggcgactaacatggctaaactttgggatcttcctgttctgttcgtttgcgaaaacaatggattcggaatgggaactactgctgaacgttcatctgcttctaccgagtattatactagaggagactatgttcctggaatctgggttgatggtatggacattctcgctgttcgcgaggctaccaagtgggctaaggaatattgtgacagtggtaaaggaccacttatgatggaaatggccacctatagataccatggacactctatgtctgacccaggaaccagctaccgtacccgtgaagagattcaagaagttcgtaaaacaagagacccaattactggattcaaggaccgtatcattacatcttcattggccacagaggaagaacttaaggctattgacaaggaagttcgcaaagaggtcgatgaagcgttgaagattgctacatcagacggtgttctcccaccagaagctctctacgccgatatttatcacaacaccccagctcaagagattcgcggagccactattgatgaaactatcgtccagccattcaaaacttctgctgatgtactgaagtctattggaagagcttaa

SEQ ID NO: 38 - protein, 397 aa, Caenorhabditis elegans
MSLFARQLQSLTASGIRTQQVRLASTEVSFHTKPCKLHKLDNGPNTSVTLNREDALKYYRDMQVIRRMESAAGNLYKEKKIRGFCHLYSGQEACAVGMKAAMTEGDAVITAYRCHGWTWLLGATVTEVLAELTGRVAGNVHGKGGSMHMYTKNFYGGNGIVGAQQPLGAGVALAMKYREQKNVCVTLYGDGAANQGQLFEATNMAKLWDLPVLFVCENNGFGMGTTAERSSASTEYYTRGDYVPGIWVDGMDILAVREATKWAKEYCDSGKGPLMMEMATYRYHGHSMSDPGTSYRTREEIQEVRKTRDPITGFKDRIITSSLATEEELKAIDKEVRKEVDEALKIATSDGVLPPEALYADIYHNTPAQEIRGATIDETIVQPFKTSADVLKSIGRA